(19) United States
(12) Patent Application Publication
(10) Pub. No.: US 2022/0145297 A1
(43) Pub. Date: May 12, 2022

(54) RNA-TARGETING CAS ENZYMES

(71) Applicant: The Regents of the University of California, Oakland, CA (US)

(72) Inventors: Anna Buchman, La Jolla, CA (US); Omar Akbari, La Jolla, CA (US)

(21) Appl. No.: 17/426,467

(22) PCT Filed: Jan. 29, 2020
(86) PCT No.: PCT/US2020/015680

§ 371 Date: Jul. 28, 2021

Related U.S. Application Data
(60) Provisional application No. 62/798,078, filed on Jan. 29, 2019.

(51) Int. Cl. C12N 15/113; C12N 9/22; C12N 15/11

(52) U.S. Cl. CPC C12N 15/113; C12N 9/22; C12N 2310/51; C12N 2310/20; C12N 15/111

(57) ABSTRACT

Provided herein are compositions and methods for CRISPR based RNA-targeting. The compositions include nucleic acid molecules comprising a sequence encoding a Cas13 polypeptide and a sequence encoding a guide RNA comprising one or more spacers and one or more Cas13-specific direct repeats, where the spacers are capable of specifically hybridizing with one or more target RNAs. The disclosure further provides methods of modifying a target RNA in a cell and transgenic organisms.

### CROSS-REFERENCE TO RELATED APPLICATIONS

This application claims priority to U.S. Patent Application Ser. No. 62/798,078, filed Jan. 29, 2019, which is incorporated herein by reference in its entirety.

### STATEMENT OF GOVERNMENT SUPPORT

This invention was made with government support under HR0011-17-2-0047 awarded by the Defense Advanced Research Project Agency. The government has certain rights in the invention.

### BACKGROUND

The development of CRISPR as a programmable genome-engineering tool provides transformative applications for both medicine and biotechnology. However, much of the recent focus in synthetic biology has been on exploiting CRISPR to target DNA. Improved compositions and methods for utilizing CRISPR to target RNA are therefore needed.

### SUMMARY

In one aspect, provided herein are nucleic acid molecule comprising: (a) a sequence encoding a Cas13 polypeptide; and (b) a sequence encoding a guide RNA comprising one or more spacers and one or more Cas13-specific direct repeats, wherein the spacers are capable of specifically hybridizing with one or more target RNAs. In some embodiments, the Cas13 is Cas13d. In some embodiments, the Cas13d is RfxCas13d. In some embodiments, the sequence encoding the Cas13 polypeptide further comprises a localization signal. In some embodiments, the localization signal is a nuclear localization signal. In some embodiments, the target RNA is an endogenous RNA or a viral RNA. In some embodiments, the target RNA is an mRNA. In some embodiments, the spacers are positioned between two Cas13-specific direct repeats. In some embodiments, the spacers are 20 to 40 nucleotides in length. In some embodiments, the spacers are 25 to 35 nucleotides in length. The nucleic acid molecule of claim 10, wherein the spacers are about 30 nucleotides in length. In some embodiments, the Cas13-specific direct repeats are 25 to 45 nucleotides in length. The nucleic acid molecule of claim 12, wherein the Cas13-specific direct repeats are 30 to 40 nucleotides in length. The nucleic acid molecule of claim 13, wherein the Cas13-specific direct repeats are about 36 nucleotides in length. In some embodiments, the guide RNA further comprises a AAAAC motif at its 5′ end. In some embodiments, the guide RNA comprises two or more spacers, and wherein the two or more spacers are capable of specifically hybridizing with the same target RNA. In some embodiments, the guide RNA comprises two or more spacers, and wherein the two or more spacers are capable of specifically hybridizing with different target RNAs. In some embodiments, the guide RNA comprises three or more spacers. In some embodiments, the sequence encoding a Cas13 polypeptide is operably linked to a ubiquitous promoter. In some embodiments, the sequence encoding a Cas13 polypeptide is operably linked to an inducible promoter. In some embodiments, the sequence encoding a Cas13 polypeptide is operably linked to a tissue-specific promoter. In another aspect, provided herein are vectors comprising any of the nucleic acid molecules described herein. In some embodiments, the vector is a single vector. In some embodiments, the vector is an Adeno-associated viral vector. Also provided herein are cells comprising any of the nucleic acid molecules described herein. In another aspect, provided herein are methods of modifying a target RNA in a cell, the methods comprising contacting the cell with any of the nucleic acid molecules described herein. Also provided herein are methods of modifying a target RNA in a cell, the method comprising contacting the cell with any of the vectors described herein. In some embodiments, the target RNA is endogenous RNA or viral RNA.

In another aspect, provided herein are methods of modifying a target RNA in a cell, the methods comprising contacting the cell with (a) a nucleic acid molecule comprising a sequence encoding a Cas13 polypeptide, and (b) a sequence encoding a guide RNA comprising one or more spacers and one or more Cas13-specific direct repeats, wherein the one or more spacers are capable of specifically hybridizing with the target RNA. In some embodiments, the Cas13 is Cas13d. In some embodiments, the Cas13d is RfxCas13d. In some embodiments, the sequence encoding the Cas13 polypeptide further comprises a localization signal In some embodiments, the localization signal is a nuclear localization signal. In some embodiments, the spacers are positioned between two Cas13-specific direct repeats. In some embodiments, the spacers are 20 to 40 nucleotides in length. In some embodiments, the spacers are 25 to 35 nucleotides in length. In some embodiments, the spacers are about 30 nucleotides in length. In some embodiments, the Cas13-specific direct repeats are 25 to 45 nucleotides in length. In some embodiments, the Cas13-specific direct repeats are 30 to 40 nucleotides in length. In some embodiments, the Cas13-specific direct repeats are about 36 nucleotides in length. In some embodiments, the guide RNA further comprises a AAAAC motif at its 5′ end. In some embodiments, the guide RNA comprises two or more spacers, and wherein the two or more spacers are capable of specifically hybridizing with the same target RNA. In some embodiments, the guide RNA comprises two or more spacers, and wherein the two or more spacers are capable of specifically hybridizing with different target RNAs. In some embodiments, the guide RNA comprises three or more spacers. In some embodiments, the sequence encoding a Cas13 polypeptide is operably linked to a ubiquitous promoter. In some embodiments, the sequence encoding a Cas13 polypeptide is operably linked to an inducible promoter. In some embodiments, the sequence encoding a Cas13 polypeptide is operably linked to a tissue-specific promoter. In some embodiments, the nucleic acid molecule is comprised within a first vector and the guide RNA is comprised within a second vector. In some embodiments, the first vector and/or the second vector is an AAV vector.

In another aspect, provided herein are transgenic organisms having a recombinant nucleic acid molecule stably integrated into the genome of the organisms, wherein the recombinant nucleic acid molecule comprises a sequence that encodes a Cas13 polypeptide. Also provided are transgenic organisms having two or more recombinant nucleic acid molecules stably integrated into the genome of the organism, comprising at least a first recombinant nucleic acid molecule that comprises a sequence encoding a Cas13 polypeptide, and a second recombinant nucleic acid molecule that comprises a sequence that encodes a guide RNA. In some embodiments, the Cas13 polypeptide is a Cas13d. In some embodiments, the Cas13d polypeptide is RfxCas13d. In some embodiments, the organism is a vertebrate. In some embodiments, the organism is an invertebrate. In some embodiments, the organism is an insect.

All publications, patents, patent applications, and information available on the internet and mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### BRIEF DESCRIPTION OF THE DRAWINGS

FIG. 1 is a schematic representation of constructs generated for the experiments described herein. All constructs used are depicted here along with addgene ID, insertion site, and Bloomington stock number.

FIGS. 2A-2C show genetic assessment of programmable CasRx-mediated transcript knockdown in flies. FIG. 2A is a representative genetic crossing schematic for generating transhetrozygotes. FIG. 2B shows inheritance and penetrance rates of transheterozygous flies inheriting both Ubiq-CasRx, or Ubiq-dCasRx, and gRNA^{array} corresponding to the box in FIG. 2A. Phenotype penetrance rate is depicted by shading in the box plot. FIG. 2C shows brightfield images of transheterozygous flies with representative phenotypes for each cross. Corresponding genotype for each image is dictated by the combination of constructs on top of the columns and the side of the rows.

FIG. 3 shows the complete inheritance plot of bidirectional crosses featured in FIG. 2B.

FIGS. 4A and 4B show development related inheritance and lethality of Ubiq-CasRx and Ubiq-dCasRx transheterozygotes. FIG. 4A shows transheterozygote percentages at larval, pupal, and adult development periods for each gRNA^{array} producing an observable phenotype (w, cn, wg). FIG. 4B shows transheterozygote percentages through larval, pupal, and adult development periods for each gRNA^{array} producing a lethal phenotype (N, y, GFP).

FIGS. 5A-5C show CasRx-mediated transcript knockdown in restricted tissue types using the binary Gal4/UAS system. FIG. 5A shows representative genetic crossing schematic demonstrating the two steps followed in each generational cross. FIG. 5B shows inheritance rates of triple transheterozygous flies inheriting 3 transgenes (UASt-CasRx or UASt-dCasRx, gRNA^{array}, and Gal4-driver), corresponding to flies highlighted in the box in FIG. 5A. FIG. 5C are image matrix of the triple transheterozygous flies inheriting 3 transgenes. The identities of inherited transgenes for each triple transheterozygote is specified through combination of the top and left side labels of the image matrix. The black arrow identifies tissue necrosis and pigment reduction observed from cn targeting. The white arrow identifies chitin pigment reduction in the thorax resulting from y targeting. Black and white fly with “X” represents a lethal phenotype with no live adults able to be scored or imaged.

FIG. 6 shows complete inheritance data for binary Gal4/UAS crosses.

FIGS. 7A-7D show genetic assessment of CasRx-mediated transcript cleavage and subsequent lethality. FIG. 7A is a representative genetic crossing schematic used to obtain triple transheterozygotes (box) for luciferase expression analysis. FIG. 7B shows total inheritance percentages for all genotypes emerging in F₂ generation. FIG. 7C shows inheritance of Ubiq-CasRx/gRNA^{Fluc} or Ubiq-CasRx/Ubiq-Fluc-Ubiq-Rluc, and inheritance ratios between Ubiq-CasRx and Ubiq-dCasRx transheterozygotes. FIG. 7D shows luciferase ratios normalizing Fluc readings to Rluc readings.

FIG. 8 shows dual-luciferase reporter system transgenic markers, with representative markers for each construct.

FIGS. 9A-9C show CasRx-mediated knockdown of GFP. FIG. 9A shows a representative bidirectional genetic crossing schematic. FIG. 9B shows a box plot of transheterozygote inheritance resulting from bidirectional crosses between Ubiq-CasRx or Ubiq-dCasRx and gRNA^{GFP}-OpIE2-GFP flies (M=maternal inheritance of CasRx; P=paternal inheritance of CasRx). FIG. 9C are images of F₁ larvae from paternal crosses clearly demonstrating significant reduction in GFP expression for transheterozygous larvae expressing both Ubiq-CasRx and gRNA^{GFP}-OpIE2-GFP compared to control transheterozygotes expressing Ubiq-dCasRx and gRNA^{GFP}-OpIE2-GFP or without expressing a CasRx protein. (Left-right) Ubiq-CasRx/gRNA^{GFP} transheterozygous larvae, heterozygous gRNA^{GFP} larvae from Ubiq-CasRx cross, Ubiq-dCasRx/gRNA^{GFP} transheterozygous larvae, heterozygous gRNA^{GFP} larvae from Ubiq-dCasRx cross.

FIG. 10 shows modENCODE transcript expression relative to Drosophila melanogaster development. Black box indicates which developmental period was chosen for RNA sequencing of samples for analysis of CasRx-mediated transcript knockdown in Ubiq-CasRx vs Ubiq-dCasRx comparison.

FIGS. 11A-11C show quantification of CasRx-mediated on/off target activity. FIG. 11A shows maximum a posteriori (MAP) estimates for the logarithmic fold change (LFC) of transcripts. Grey dots represent transcripts not significantly differentially expressed between Ubiq-CasRx and Ubiq-dCasRx group (p>0.05). Red dots represent transcripts significantly differentially expressed between CasRx and dCasRx group (p<0.05). Pink dot identifies the respective CasRx target gene for each analysis (p value indicated in the inset). FIG. 11B shows transcript expression levels (TPM) of transcripts targeted with CasRx or dCasRx. FIG. 11C shows percentage of transcripts significantly differentially expressed resulting from CasRx cleavage.

FIGS. 12A and 12B are schematic diagrams showing CasRx-gRNA^{array} transcript target selection and construct generation. FIG. 12A is a schematic representing the workflow for gRNA choice. FIG. 12B is a schematic diagram showing the generation of gRNA^{array} construct.

FIG. 13 shows schematic diagrams of transcriptome engineering with RNA-targeting Type VI-D CRISPR effectors and CRISPR-Cas13 precision transcriptome engineering in cancer.

FIG. 14 shows mutant phenotypes in the eye and wing of D. melanogaster induced by RfxCas13d and pre-crRNA arrays targeting D. melanogaster notch (CG3936) and white (CG2759) genes.

FIG. 15 is a schematic diagram showing engineered pan-antiviral effector cassettes that can target multiple RNA viruses transmitted by mosquitoes, including Zika, chikungunya, dengue fever, and yellow fever viruses.

### DETAILED DESCRIPTION

The present disclosure provides nucleic acid molecules comprising (a) a sequence encoding a Cas13 polypeptide; and (b) a sequence encoding a guide RNA comprising one or more spacers and one or more Cas13-specific direct repeats, where the spacers are capable of specifically hybridizing with one or more target RNAs. In some instances, the Cas13 is Cas13d. Also provided are methods of modifying a target RNA in a cell comprising contacting the cell with a nucleic acid molecule comprising a sequence encoding a Cas13 polypeptide and a sequence encoding a guide RNA described herein. The present disclosure further provides transgenic organisms having a recombinant nucleic acid molecule stably integrated into the genome of the organism, where the recombinant nucleic acid molecule comprises a sequence that encodes a Cas13 polypeptide.

Current applications of CRISPR-Cas nucleases in Drosophila melanogaster are limited to DNA-targeting class 2 systems. The present disclosure reports, among other things, a programmable platform for transcript targeting applications utilizing a Type VI-D RNA-targeting Cas ribonuclease, CasRx. The present disclosure provides methods for genetically encoding CasRx allowing for CRISPR-based transcript targeting manifesting as visible phenotypes comparable to previous gene knockdown experiments. Through genetic and bioinformatic analysis, the disclosure demonstrates on-target transcript knockdown capabilities of CasRx. The disclosure also includes description of off-target effects following on-target transcript cleavage by CasRx, providing the first evidence of off-target activity expressing a Type VI ribonuclease in eukaryotes. The disclosure provides the use of a programmable RNA-targeting Cas system in e.g., Drosophila melanogaster, and provides alternative approaches for in vivo gene knockdown studies.

CRISPR functions via the association of CRISPR RNAs (crRNAs) and CRISPR-associated (Cas) proteins to provide adaptive and heritable immunity to protect prokaryotic hosts from foreign genetic elements and invading viruses. Specifically, it acts as a programmable RNA-guided nuclease capable of degrading exogenous nucleic acids (DNA or RNA) by exploiting molecular memory of prior infections archived as heritable DNA sequences in CRISPR arrays. These CRISPR arrays consist of altering repeats and invader-derived (spacer) DNA sequences which get transcribed and then processed into small, mature crRNAs. Mature crRNAs then combine with Cas proteins to form crRNA-Cas complexes, which target and cleave specific nucleic acid sequences. There are several types and subtypes of CRISPR systems found in bacteria that utilize a diversity of proteins and mechanisms to provide immunity. For example, Type I, II, V (and perhaps IV) target DNA, while Type III targets both DNA and RNA, and Type VI targets RNA exclusively.

While much of the recent focus in synthetic biology has been on exploiting CRISPR to target DNA, the recent findings that Type VI CRISPR systems can also be reprogrammed to target RNA has revealed exciting possibilities for transcriptome engineering. For example, one recent discovery was the finding and functional characterization of CasRx as a compact single-effector Cas enzyme that exclusively targets RNA with superior efficiency and specificity as compared to RNA interference (RNAi) (See e.g., Konermann et al. Cell 173:665-676 (2018)). In human cells, CasRx demonstrated highly efficient on-target gene knockdown with limited off-target activity. Given these characteristics, we wanted to test its functionality in Drosophila melanogaster (flies) to enable the exploration of new biological questions in vivo. While CRISPR has been used extensively to generate heritable DNA mutations in flies, RNA-targeting using CRISPR has not been demonstrated and therefore RNA-targeting in flies is restricted to the application of RNAi-based approaches.

The present disclosure provides the first use of a Cas-based RNA-targeting system through CasRx-mediated transcript targeting in vivo, e.g., in flies. In some instances, the methods and compositions provided herein involve CasRx and guide RNA arrays (gRNA^{array}) that are encoded in the genome to promote robust expression throughout development. Performing bidirectional and binary genetic crosses with ubiquitous and tissue-specific expression of CasRx, the disclosure demonstrates the ability to obtain clear, highly penetrant phenotypes comparable to previously established phenotypes obtained by RNAi. In some instances, transcript knockdown are quantified through RNA sequencing (RNAseq) analysis. CasRx is shown to be capable of targeted knockdown for various genes at numerous stages of fly development, and can be useful for transcript targeting applications and genome editing in vivo.

Unless otherwise indicated “nuclease” can refer to an enzyme which possesses endonucleolytic catalytic activity for polynucleotide cleavage.

A “target RNA” as used herein can include an RNA that can include a “target sequence”. The term “target sequence” can refer to a nucleic acid sequence present in a target RNA to which a spacer of a guide RNA can hybridize, provided sufficient conditions for hybridization exist. Hybridization between the spacer and the target sequence can, for example, be based on Watson-Crick base pairing rules, which enables programmability in the spacer sequence. The spacer sequence can be designed, for instance, to hybridize with any target sequence.

The “spacer” within a guide RNA can include a nucleotide sequence that is complementary to a specific sequence within a target RNA.

“Binding” as used herein can refer to a non-covalent interaction between macromolecules (e.g., between a protein and a nucleic acid). While in a state of non-covalent interaction, the macromolecules are said to be “associated” or “interacting” or “binding” (e.g., when a molecule X is said to interact with a molecule Y, it means that the molecule X binds to molecule Y in a non-covalent manner). Binding interactions are generally characterized by a dissociation constant (Kd) of less than 10⁻⁶ M, less than 10⁻⁷ M, less than 10⁻⁸ M, less than 10⁻⁹ M, less than 10⁻¹⁰ M, less than 10⁻¹¹ M, less than 10⁻¹² M, less than 10⁻¹³ M, less than 10⁻¹⁴ M, or less than 10⁻¹⁵ M. Kd is dependent on environmental conditions, e.g., pH and temperature, as is known by those in the art. “Affinity” refers to the strength of binding, and increased binding affinity is correlated with a lower Kd.

The terms “hybridizing” or “hybridize” can refer to the pairing of substantially complementary or complementary nucleic acid sequences within two different molecules. Pairing can be achieved by any process in which a nucleic acid sequence joins with a substantially or fully complementary sequence through base pairing to form a hybridization complex. For purposes of hybridization, two nucleic acid sequences or segments of sequences are “substantially complementary” if at least 80% of their individual bases are complementary to one another.

As used herein, “operably linked” can refer to the situation in which part of a linear DNA sequence can influence the other parts of the same DNA molecule. For example, when a promoter controls the transcription of the coding sequence, it is operatively linked to the coding sequence.

As used herein, a “polypeptide” can include proteins, fragments of proteins, and peptides, whether isolated from natural sources, produced by recombinant techniques, or chemically synthesized. A polypeptide may have one or more modifications, such as a post-translational modification (such as glycosylation, etc.) or any other modification (such as PEGylation, etc.). The polypeptide may contain one or more non-naturally-occurring amino acids (such as an amino acid with a side chain modification). Polypeptides described herein typically comprise at least about 10 amino acids.

As used herein, “contacting” a cell with a nucleic acid molecule can be allowing the nucleic acid molecule to be in sufficient proximity with the cell such that the nucleic acid molecule can be introduced into the cell.

A “promoter” can be a region of DNA that leads to initiation of transcription of a gene.

A “motif” can be a nucleotide or amino acid sequence pattern that is correlated with biological significance or function.

I. Cas 13 Polypeptide

Provided herein are nucleic acid molecules comprising: (a) a sequence encoding a Cas13 polypeptide; and (b) a sequence encoding a guide RNA comprising one or more spacers and one or more Cas13-specific direct repeats, where the spacers are capable of specifically hybridizing with one or more target RNAs.

“Nucleic acid molecules” as used herein can include a DNA sequence or an RNA sequence. The Cas13 polypeptide can be any of the Cas13 polypeptides described herein or known in the art. “Cas13 polypeptides” and “Cas13” are used interchangeably herein. Cas13 are RNA-targeting programmable nucleases associated with Type VI CRISPR-Cas systems. Type VI CRISPR-Cas systems are dedicated RNA-targeting immune systems in prokaryotes. Cas13 family contains at least four known subtypes, including Cas13a (formerly C2c2), Cas13b, Cas13c and Cas13d. Type VI-A and VI-B systems possess the crRNA-dependent target cleavage activity and a non-specific, collateral RNase activity that is stimulated by target recognition and cleavage. Both of these activities are mediated by the two HEPN domains contained in type VI effectors Cas13a and Cas13b (Yan et al. Molecular Cell 70(2):327-339, 2018).

In some instances, the Cas13 is a Cas13d protein. Cas13d are effectors associated with subtype VI-D, a variant of type VI CRISPR-Cas, and have robust target cleavage and collateral RNase activities along with their ability to process pre-crRNA. Cas13d has a smaller size compared to other Cas13s and can be advantageous for RNA targeting applications described herein, such as for packaging into a viral vector for delivery.

Cas13 can be guided by a guide RNA which encodes target specificity. The Cas13 protein complexes with the guide RNA via recognition of a short hairpin in the crRNA and target specificity is encoded by a spacer that is complementary to the target region. In addition to programmable RNase activity, Cas13s exhibit collateral activity after recognition and cleavage of a target transcript, leading to non-specific degradation of any nearby transcripts regardless of complementarity to the spacer. Cas13 can process its own pre-crRNAs, allowing individual short single crRNAs to be customized to target RNA in vitro or to provide Escherichia coli with programmable immunity against the lytic single-stranded RNA MS2 bacteriophage. CRISPR/Cas13 can have broad applicability as an RNAi-like platform for RNA silencing. Compared to small RNAs and RNA interference, which are difficult in design and are limited by high off-target potential, CRISPR/Cas13 can be used to manipulate only the target RNA, with few or no off-target effects in eukaryotes, and multiple crRNAs can be used to eradicate a particular mRNA transcript.

The Cas13 polypeptides can be naturally-occurring or non-naturally occurring. The Cas13 polypeptides can be a mutant Cas13 polypeptide (e.g., a mutant of a naturally occurring Cas13 polypeptide). Mutant Cas13 can have altered activity compared to a naturally occurring Cas13, such as altered nuclease activity without substantially diminished binding affinity to RNA). In some instances, the mutant Cas13 has no nuclease (e.g., ribonuclease) activity. For instance, mutant Cas13 encodes a ribonuclease with the positively charged catalytic residues of the HEPN motifs inactivated thereby eliminating programmable RNA cleavage without affecting guide RNA array processing, or target RNA binding. The Cas13 can have a size of about 700 to about 1200 amino acids (e.g., about 700 to about 1100, about 700 to about 1000, about 700 to about 900, about 700 to about 800, about 800 to about 1200, about 800 to about 1100, about 800 to about 1000, about 800 to about 900, about 900 to about 1200, about 900 to about 1100, about 900 to about 1000, about 1000 to about 1200, about 1000 to about 1100, or about 1100 to about 1200 amino acids). In some instances, the Cas13 has a size of about 930 amino acids. In some instances, the Cas13 is Cas13d. Cas13d derived from a variety of species are contemplated herein, including but not limited to, Ruminococcus sp., Ruminoccocus flavefaciens, Ruminoccocus albus, and Eubacterium siraeum. In some instances, the Cas13d is derived from Ruminococcus flavefaciens strain XPD3002 (e.g., CasRx or RfxCas13d). In some instances, the Cas13d is a catalytically inactive version of CasRx (e.g. dCasRx). An exemplary sequence of CasRx (NLS-RfxCas13d-NLS) can be found at Plasmid #109049 (pXR001: EF1a-CasRx-2A-EGFP, addgene).

The sequence encoding a Cas13 polypeptide described herein can be at least 80% identical (e.g. at least 82%, 84%, 86%, 88%, 90%, 92%, 94%, 96%, 98%, or 99% identical) to the sequence of RfxCas13d. In some instances, the sequence encoding a Cas13 polypeptide is identical to the sequence of Cas13d.

In some embodiments, the nucleic acid molecule provided herein comprises a sequence encoding a Cas13 protein and further comprises one or more localization signals. Localization signals can be an amino acid sequence on a protein that tags the protein for transportation to a particular location in a cell. An exemplary localization signal is nuclear localization signal, which can be an amino acid sequence that tags a protein for import into the cell nucleus by nuclear transport. The localization signals can be operably linked to the sequence encoding a Cas13 protein. In some embodiments, the localization signal is a nuclear localization signal. For example, the sequence encoding Cas13 can encode two nuclear localization signals, where upon translation, the Cas13 is fused to N- and C-terminal nuclear localization signals. An exemplary NLS is SV40 large T antigen NLS (PKKKRRV (SEQ ID NO: 1)) and nucleoplasmin NLS (KRPAATKKAGQAKKKK (SEQ ID NO: 2)). Other NLSs are known in the art; see, e.g., Konermann et al., Cell 173:665-676, 2018; Cokol et al., EMBO Rep. 1(5):411-415 (2000); Freitas and Cunha, Curr Genomics 10(8): 550-557 (2009).

In some instances, the sequence encoding a Cas13 polypeptide is operably linked to a promoter. Suitable promoters include but are not limited to ubiquitous promoters (e.g., ubiquitin promoter), tissue-specific promoters, inducible promoters, and constitutive promoters.

The sequence encoding a Cas13 polypeptide can be further operably linked to a sequence that encodes one or more reporter genes. Any suitable reporter genes are contemplated, including but not limited to, fluorescent reporters.

II. Guide RNA

Provided herein are guide RNAs comprising one or more spacers and one or more Cas13-specific direct repeats, where the spacers are capable of specifically hybridizing with one or more target RNAs. Also provided herein are sequences encoding the guide RNAs provided herein.

The guide RNA can include at least one (e.g., at least two, three, four, five, six, or seven) spacers. The spacers can bind to the same or different target sequences in the same target RNA, or can bind to different target RNAs. The spacers can be designed to target any sequence in a target RNA. In instances where two or more spacers are included in the guide RNA, the spacers can have the same or different length. The spacers can have a length of between 20 to 40 nucleotides (e.g., 20 to 35, 20 to 30, 20 to 25, 25 to 40, 25 to 35, 25 to 30, 30 to 40, 30 to 35, or 35 to 40 nucleotides). In some instances, the spacers can have a length of about 30 nucleotides.

The guide RNA can include at least one (e.g., at least two, three, four, five, six, or seven) direct repeats. A direct repeat can be a repetitive sequence within a CRISPR locus that are interspersed by short spacers. A direct repeat sequence can have homology to a trans-activating CRISPR RNA, and facilitates the formation of a crRNA: tracrRNA duplex. The sequence and secondary structure of Cas13-specific direct repeats can be dependent on the specific Cas13. For instance, Cas13d from different species can have different direct repeat sequences and/or secondary structures. Exemplary direct repeat sequences for Cas13d can be found at e.g. Konnerman et al. Cell 173:665-676 (2018). The Cas13-specific direct repeats in the guide RNA provided herein can be chosen based on the specific Cas13 used. Direct repeat sequences functioning together with Cas13 proteins of various bacterial species may be identified by bioinformatic analysis of sequence repeats occurring in the respective CRISPR/Cas operons and by experimental binding studies of Cas13 protein together with putative DR sequence flanked target sequences. The Cas13-specific direct repeats can be about 30 to about 40 (e.g., about 31, 32, 33, 34, 35, 36, 37, 38, or 39) nucleotides in length. In some instances, the Cas13-specific (e.g., Cas13d-specific) direct repeats are about 36 nucleotides in length. In some instances, the direct repeats form a hairpin structure capable of interacting with the Cas13 polypeptide to form a complex. In some instances, the Cas13-specific direct repeats are Cas13d-specific direct repeats. Exemplary Cas13d-specific direct repeat sequences can be found at Konermann et al. Cell 173:665-676 (2018).

An exemplary sequence of a RfxCas13d-specific direct repeat is shown below (SEQ ID NO: 3): CAAGUAAACCCCUACCAACUGGUCGGGGUUUGAAAC

The direct repeats in the guide RNA described herein can include a sequence that is at least 80% identical (e.g. at least 82%, 84%, 86%, 88%, 90%, 92%, 94%, 96%, 98%, 99% identical) to SEQ ID NO: 3.

The spacers can be arranged in tandem and interspersed by direct repeats. For example, a spacer can be positioned between two direct repeats. The guide RNA can include, e.g., as part of its sequence, [direct repeat 1-spacer 1-direct repeat 2-spacer 2-direct repeat 3-spacer 3-direct repeat 4-spacer 4-direct repeat 5]. In some instances, the guide RNA includes n spacers and n+1 direct repeats, where n≥1 (e.g., 1, 2, 3, 4, 5, 6, 7, 8, 9 or 10).

Some embodiments disclosed herein provide nucleic acid molecules that encode the guide RNA. Some embodiments provide vectors comprising nucleic acid molecules encoding the guide RNA. Nucleic acid molecules encoding the guide RNA can be operably linked to one or more promoters. Any suitable promoters described herein and known in the art are contemplated, such as but not limited to, a polymerase III promoter, such as a polymerase-3 U6 (U6:3) promoter. Exemplary U6 promoters can be found e.g., in Xia et al. Nucleic Acids Res. 31(17) e100; or at Addgene plasmid #112688 (gRNA[Sxl]0.1026B).

Nucleic acid molecules encoding the guide RNA can be further operably linked to sequences that encode one or more reporter genes. Any suitable reporter genes are contemplated, including but not limited to, fluorescent reporters.

III. Vectors

Some embodiments disclosed herein provide vectors (e.g. viral vectors) that comprise nucleic acid molecules comprising a sequence encoding a Cas13 polypeptide (e.g. any Cas13 polypeptides described herein) and/or a sequence encoding a guide RNA (e.g. any guide RNAs described herein). Any suitable vectors described herein and known in the art are contemplated. In some instances, the viral vector is an Adeno-associated viral vector (AAV). Adeno-associated virus is a naturally occurring defective virus that requires another virus, such as an adenovirus or a herpes virus, as a helper virus for efficient replication and a productive life cycle. (For a review, see Muzyczka et al., Curr. Topics in Micro and Immunol. 158:97-129 (1992)). AAV vectors efficiently transduce various cell types and can produce long-term expression of transgenes in vivo. AAV vectors have been extensively used for gene augmentation or replacement and have shown therapeutic efficacy in a range of animal models as well as in the clinic; see, e.g., Mingozzi and High, Nature Reviews Genetics 12, 341-355 (2011); Deyle and Russell, Curr Opin Mol Ther. 2009 August; 11(4): 442-447; Asokan et al., Mol Ther. 2012 April; 20(4): 699-708. AAV vectors containing as little as 300 base pairs of AAV can be packaged and can produce recombinant protein expression. For example, AAV2, AAV5, AAV2/5, AAV2/8 and AAV2/7 vectors have been used to introduce DNA into photoreceptor cells (see, e.g., Pang et al., Vision Research 2008, 48(3):377-385; Khani et al., Invest Ophthalmol Vis Sci. 2007 September; 48(9):3954-61; Allocca et al., J. Virol. 2007 81(20):11372-11380). In some embodiments, the AAV vector can include (or include a sequence encoding) an AAV capsid polypeptide described in PCT/US2014/060163; for example, a virus particle comprising an AAV capsid polypeptide having an amino acid sequence selected from the group consisting of SEQ ID NOs: 1, 3, 5, 7, 9, 11, 13, 15, and 17 of PCT/US2014/060163, and a Cas9 sequence and guide RNA sequence as described herein. In some embodiments, the AAV capsid polypeptide is an Anc80 polypeptide, e.g., Anc80L27; Anc80L59; Anc80L60; Anc80L62; Anc80L65; Anc80L33; Anc80L36; or Anc80L44. In some embodiments, the AAV incorporates inverted terminal repeats (ITRs) derived from the AAV2 serotype. Exemplary left and right ITRs are presented in Table 6 of WO 2018/026976. It should be noted, however, that numerous modified versions of the AAV2 ITRs are used in the field, and the ITR sequences shown below are exemplary and are not intended to be limiting. Modifications of these sequences are known in the art, or will be evident to skilled artisans, and are thus included in the scope of this disclosure. Expression of Cas13 and/or guide RNA in the AAV vector can be driven by a promoter described herein or known in the art.

IV. Target RNA and Methods of Modifying a Target RNA in a Cell

The target RNA can be any RNA molecules endogenous or exogenous to a eukaryotic cell, and can be protein-coding or non-protein-coding. A variety of RNA targets are contemplated herein. For example, a target RNA can be messenger RNA (mRNA), ribosomal RNA (rRNA), signal recognition particle RNA (SRP RNA), transfer RNA (tRNA), small nuclear RNA (snRNA), small nucleolar RNA (snoRNA), antisense RNA (aRNA), long noncoding RNA (lncRNA), microRNA (miRNA), piwi-interacting RNA (piRNA), small interfering RNA (siRNA), short hairpin RNA (shRNA), retrotransposon RNA, viral genome RNA, viral noncoding RNA, or the like. A guide RNA provided herein can include spacers that are capable of specifically hybridizing with the same target RNA or at least two different target RNAs.

In some instances, the RNA can be a viral RNA (e.g., single stranded viral RNA). The viral RNA can be from an anthropod-borne virus (arboviruse), such as but not limited to tick-borne viruses, midge-borne viruses, and mosquito-borne viruses. Exemplary viruses include, but are not limited to, Zika, Chikungunya, Dengue, Yellow fever, West Nile, Japanese encephalitis, Rift Valley fever, and Eastern equine encephalitis viruses. See, e.g. Reynolds et al. Comp Med 67(3):232-241 (2017). Additional viruses contemplated include, but are not limited to, lymphocytic choriomeningitis virus (LCMV), influenza A virus (IAV), and vesicular stomatitis virus (VSV). Additional viral RNAs that can be targeted by the compositions and methods described here in can be found at e.g., Frejie et al., Molecular Cell, 76(5):826-837. Collateral cleavage and tissue-specific cell death resulting from the use of the systems provided herein can be useful for ssRNA virus targeting in arbovirus vectors.

In some aspects, the present disclosure provides methods of modifying a target RNA in a cell. The methods can include introducing a nucleic acid sequence encoding a Cas13 polypeptide (e.g., any of the Cas13 polypeptides described herein) and a guide RNA (e.g., any of the guide RNAs described herein) into the cell. The sequence encoding a Cas13 protein and the guide RNA can be introduced into the cell in the same nucleic acid molecule or in different nucleic acid molecules. In some instances, the methods include contacting the cell with (a) a nucleic acid molecule comprising a sequence encoding a Cas13 polypeptide, and (b) a sequence encoding a guide RNA comprising one or more spacers and one or more Cas13-specific direct repeats, wherein the one or more spacers are capable of specifically hybridizing with the target RNA. In some instances, the sequence encoding a Cas13 polypeptide is introduced via a first vector (e.g. any suitable vectors described herein) and the guide RNA is introduced via a second vector (e.g. any suitable vectors described herein). Also contemplated are cells comprising the nucleic acid molecule comprising a sequence encoding a Cas13 polypeptide and/or a sequence encoding a guide RNA described herein.

Methods of Monitoring Target RNA Modification

The present disclosure in some instances provides compositions for and methods of monitoring target RNA modification e.g., in a cell, comprising monitoring the presence and/or levels of a target RNA, or monitoring the presence and/or levels of a protein corresponding to a target RNA (e.g. for protein-coding RNA). Any suitable techniques and assays for monitoring RNA and/or protein levels known in the art are contemplated herein. Exemplary methods include in situ hybridization, antibody staining, and RNA sequencing.

V. Transgenic Organisms

As used herein, a “transgenic organism” can include a non-human animal in which one or more of the cells of the organism includes a transgene. The organism can be a vertebrate or an invertebrate, such as an arthropod (e.g., an insect).

In some instances, a transgenic organism provided herein has a recombinant nucleic acid molecule stably integrated into the genome of the organism, wherein the recombinant nucleic acid molecule comprises a sequence that encodes a Cas13 polypeptide (e.g. any of the Cas13 polypeptides described herein). In some instances, a transgenic organism has two or more recombinant nucleic acid molecules stably integrated into the genome of the organism, comprising at least a first recombinant nucleic acid molecule that comprises a sequence encoding a Cas13 polypeptide, and a second recombinant nucleic acid molecule that comprises a sequence that encodes a guide RNA.

A founder animal carrying a recombinant nucleic acid comprising a sequence that encodes a Cas13 polypeptide can be identified based upon the presence of the sequence in its genome and/or expression of Cas13 in tissues or cells of the animal. A founder animal carrying a recombinant nucleic acid comprising a sequence that encodes a guide RNA can be identified based upon the presence of the sequence in its genome. A transgenic founder animal can then be used to breed additional animals carrying the transgene. A transgenic animal can be heterozygous or homozygous for the transgenes.

Methods for making transgenic animals are known in the art; see, e.g., WO2016049024; WO201604925; WO2017124086; WO2018009562; and U.S. Pat. No. 9,901,080. Such techniques include, without limitation, pronuclear microinjection (See, e.g., U.S. Pat. No. 4,873,191), retrovirus mediated gene transfer into germ lines (Van der Putten et al., Proc. Natl. Acad. Sci. USA, 82:6148-1652 (1985)), gene targeting into embryonic stem cells (Thompson et al., Cell 56:313-321 (1989)), electroporation of embryos (Lo, Mol. Cell. Biol., 3:1803-1814 (1983)), and in vitro transformation of somatic cells, such as cumulus or mammary cells, followed by nuclear transplantation (Wilmut et al., Nature, 385:810-813 (1997); and Wakayama et al., Nature, 394:369-374 (1998)); these methods can be modified to use CRISPR as described herein. For example, fetal fibroblasts can be genetically modified using CRISPR as described herein, and then fused with enucleated oocytes. After activation of the oocytes, the eggs are cultured to the blastocyst stage. See, for example, Cibelli et al., Science, 280:1256-1258 (1998).

The present disclosure also provides a population of cells isolated from an organism as described herein, as well as primary or cultured cells, e.g., isolated cells, engineered to include a sequence that encodes a Cas13 protein and/or a guide RNA. The cells can be isolated from any of the transgenic animals described above. Also provided are methods of introducing the transgenes described herein into a cell (e.g., primary cells or cultured cells). Exemplary methods include viral delivery (e.g., using viral vectors) and electroporation.

### EXAMPLES

### Example 1: Genetically Encoding CasRx in Flies

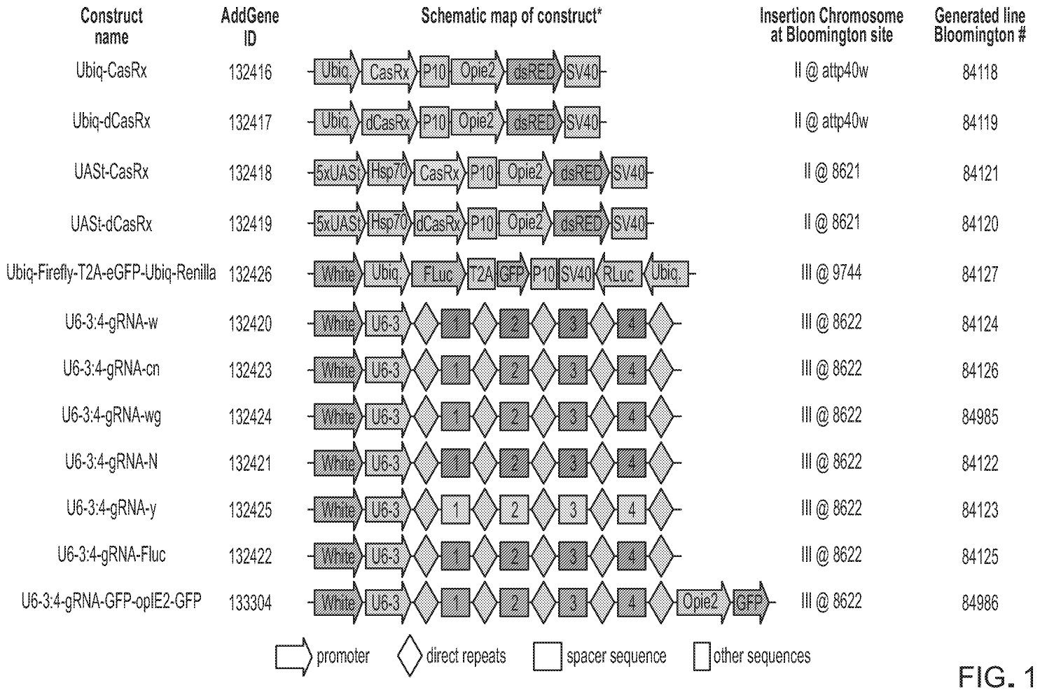

To determine the efficacy of CRISPR-based programmable RNA-targeting in flies, flies were engineered to encode the CasRx ribonuclease. To do so, two transgenes were generated utilizing a broadly expressing ubiquitin (Ubiq) promoter to drive expression of either CasRx (Ubiq-CasRx), or a catalytically inactive version of the ribonuclease, termed dCasRx (Ubiq-dCasRx), used as a negative control (FIG. 1). Importantly, dCasRx encodes a ribonuclease with the positively charged catalytic residues of the HEPN motifs inactivated thereby eliminating programmable RNA cleavage without affecting gRNA^{array} processing, or target RNA binding. Transgenic lines integrating each transgene site-specifically were established using an available ϕC31 docking site located on the 2nd chromosome (attp40w) (FIG. 1, Table 1). While these flies were viable, homozygotes were able to be generated, for neither CasRx nor dCasRx, presumably due to high levels of ubiquitous ribonuclease expression. Therefore, these stocks were maintained as heterozygotes balanced over the chromosome Curly-of-Oster (CyO) (Table 1). To genetically measure the efficacy of programmable RNA-targeting, five genes known to produce visible phenotypes when expression is disrupted were targeted, including: white (w), cinnabar (cn), wingless (wg), Notch (N), and yellow (y). To target these genes with CasRx, a gRNA^{array} was designed for each gene driven by a ubiquitously expressed polymerase-3 U6 (U6:3) promoter (FIG. 1, Table 1). Each array consisted of four ssRNA transcript-targeting spacers (30 nt in length) each positioned between CasRx-specific direct repeats (36 nt in length) with a conserved 5′-AAAAC motif designed to be processed by either CasRx or dCasRx (FIG. 1). For each gRNA^{array}, the transgene was site-specifically integrated at an available ϕC31 docking site located on the 3rd chromosome (site 8622) and a homozygous transgenic line was established (FIG. 1, Table 1). Table 1 shows transgenic fly lines used in this study. List of transgenic fly lines used in this study identifying the corresponding Addgene vector number, the Bloomington Drosophila Stock Center stock number, and the components of each integrated construct.

### Example 2: Programmable RNA-Targeting of Endogenous Target Genes

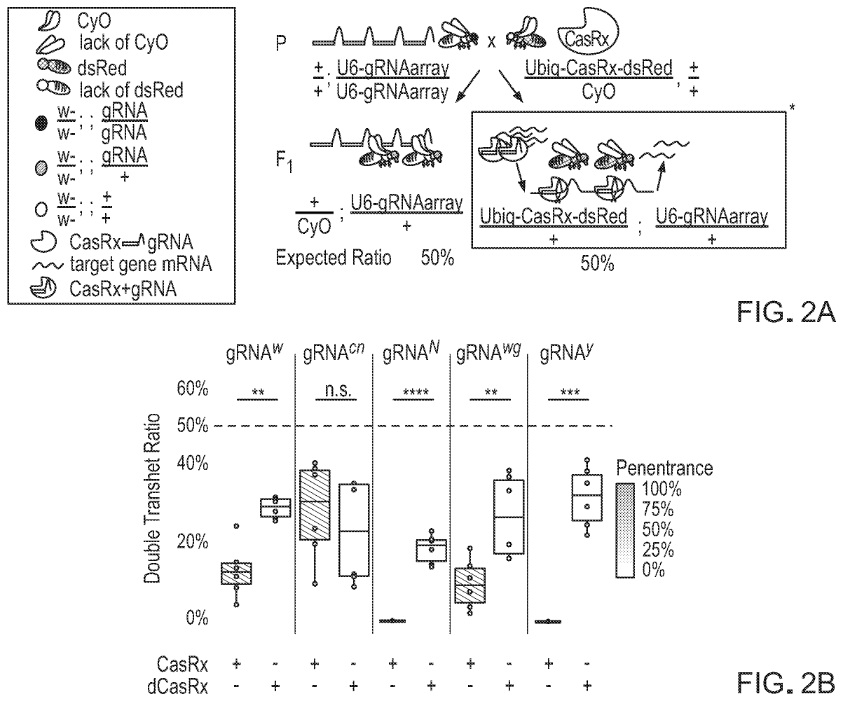

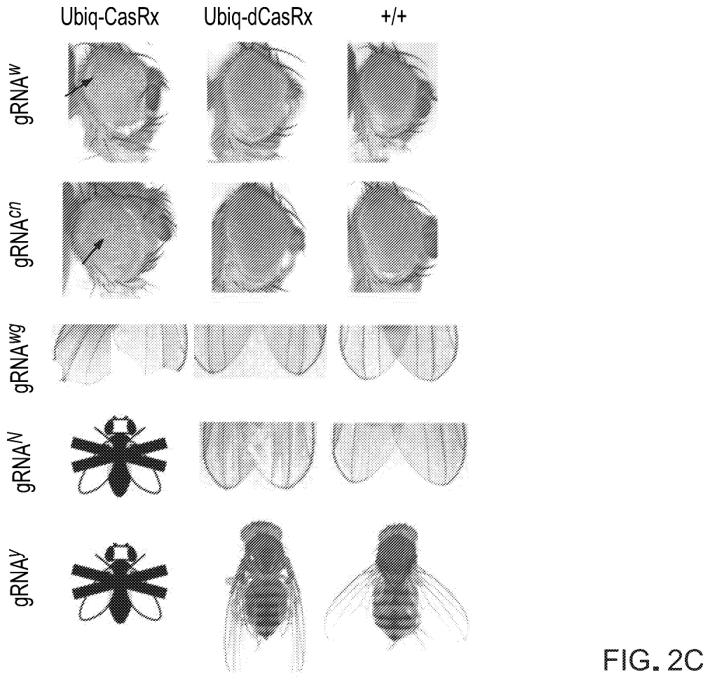

To assess the efficacy of programmable RNA-targeting by CasRx, bidirectional genetic crosses were conducted between homozygous gRNA^{array} (+/+; gRNA^{array}/gRNA^{array}) expressing flies crossed to either Ubiq-CasRx (Ubiq-CasRx/CyO; +/+), or Ubiq-dCasRx (Ubiq-dCasRx/CyO; +/+) expressing flies (FIG. 2A). Interestingly when crossed to Ubiq-CasRx, highly-penetrant (68-100%) clear visible phenotypes exclusively in transheterozygotes (Ubiq-CasRx/+; gRNA^{array}/+) for gRNA^{w}, gRNA^{cn}, and gRNA^{wg} were able to be obtained, indicating that CasRx exhibits programmable on-target RNA cleavage capabilities (FIGS. 2B and 2C, Table 2). FIG. 2B shows inheritance and penetrance rates of transheterozygous flies inheriting both Ubiq-CasRx, or Ubiq-dCasRx, and gRNA^{array} corresponding to the box in FIG. 2A. Phenotype penetrance rate is depicted by shading in the box plot. Significant differences in inheritance between CasRx and dCasRx groups were observed in 4 out of 5 groups with the exception of gRNA^{cn} (gRNA^{w}, p=0.00135; gRNA^{N}, p=0.00006; gRNA^{wg}, p=0.00851; gRNA^{y}, p=0.00016). FIG. 2C shows brightfield images of transheterozygous flies with representative phenotypes for each cross. Corresponding genotype for each image is dictated by the combination of constructs on top of the columns and the side of the rows. Clear pigment reduction is visible in both gRNA^{w} and gRNA^{cn} crosses. Arrows point to tissue necrosis in the eye where more prominent tissue necrosis is observed in gRNA^{cn} transheterozygous flies. Image for the gRNA^{wg} cross is an example of the notching phenotype resulting from incomplete development of wing margin. Black and white fly with “X” represents lethality phenotype where no transheterozygote adults emerged.

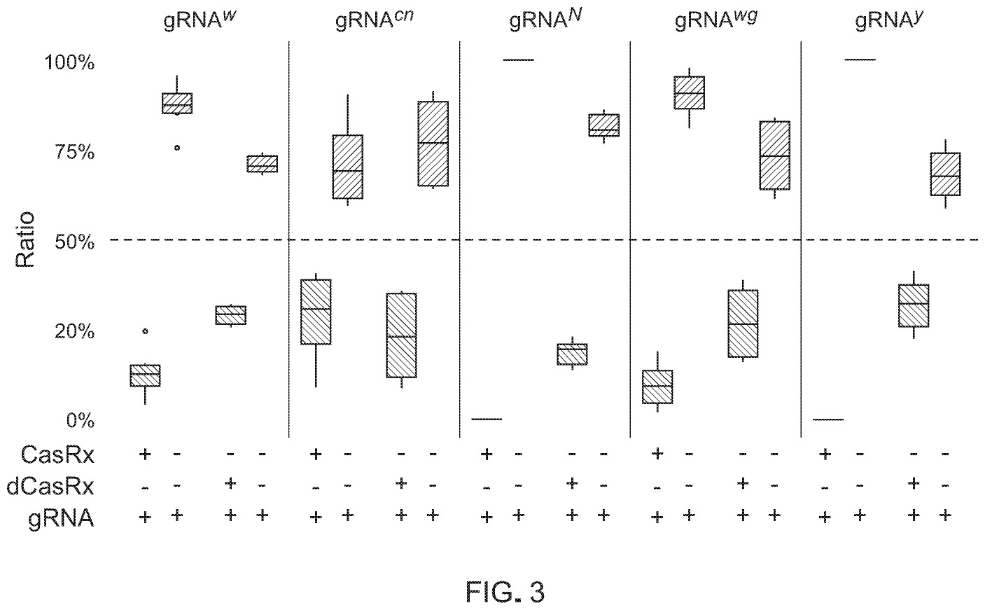

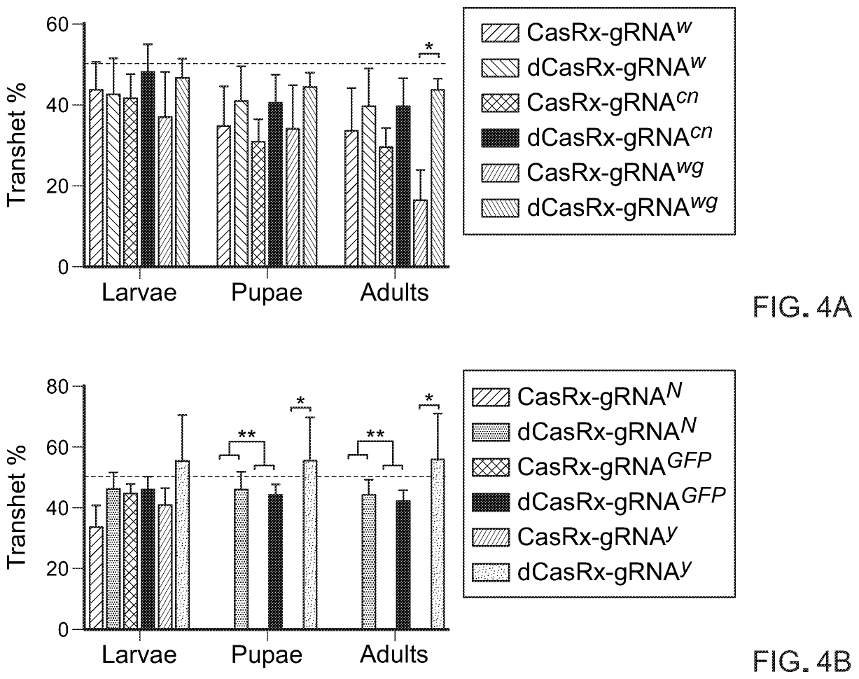

However, while the Mendelian transheterozygote inheritance rates were expected to be 50%, the recorded inheritance rates were significantly lower than expected (ranging from 9.6-28.4%) suggesting some possible toxicity leading to lethality (FIG. 2B, FIG. 3, Table 2). FIG. 3 shows the complete inheritance plot of bidirectional crosses featured in FIG. 2B. The plot includes all genotypes scored in all crosses between Ubiq-CasRx or Ubiq-dCasRx and a respective gRNA^{array}. In all crosses, gRNA^{array} only inheritance is dramatically higher than transheterozygote inheritance rates including Ubiq-dCasRx crosses. Furthermore, while the phenotypes for w, and cn resembled the expected disrupted eye pigmentation phenotypes, minor eye-specific necrosis was observed that was most prominent in Ubiq-CasRx/gRNA^{cn} transheterozygotes (FIG. 2C, arrows). Moreover when targeting wg, a notching phenotype was observed that was similar to previously observed phenotypes resulting from inhibition of wg signaling. However, when targeting y, or N, Ubiq-CasRx transheterozygotes (Ubiq-CasRx/+; gRNA^{array}/+) were 100% lethal and did not develop beyond the second instar larvae (FIGS. 4A and 4B). FIG. 4A shows transheterozygote percentages at larval, pupal, and adult development periods for each gRNA^{array} producing an observable phenotype (w, cn, wg). There were no significant differences in inheritance with the exception of gRNA^{wg} adults (p=0.014). FIG. 4B shows transheterozygote percentages through larval, pupal, and adult development periods for each gRNA^{array} producing a lethal phenotype (N, y, GFP). No Ubiq-CasRx transheterozygotes developed beyond larvae. This was expected for N as there are many examples of lethal alleles for this gene, however mutations in y should be recessive viable with defective chitin pigmentation phenotypes. Moreover, phenotypes were not obtained in transheterozygotes (Ubiq-dCasRx/+; gRNA^{array}/+) from the negative control crosses using all arrays tested, indicating that a catalytically active form of the ribonuclease is necessary for phenotypes to be observed (FIG. 2C). Taken together, these compelling genetic results indicate that the catalytically active form of the CasRx ribonuclease can generate expected phenotypes although some unexpected tissue necrosis and lethality were also observed. Table 2 shows the complete data set for the Ubiq-CasRx and Ubiq-dCasRx bidirectional crosses. Absolute counts of inheritance and phenotype penetrance for maternal and paternal inheritance of Ubiq-CasRx and Ubiq-dCasRx crosses to gRNA^{array} or Ubiq-Fluc-Ubiq-Rluc expressing flies. Each cross (paternal and maternal) was done in triplicate.

### Example 3: Tissue-Specific RNA-Targeting by CasRx

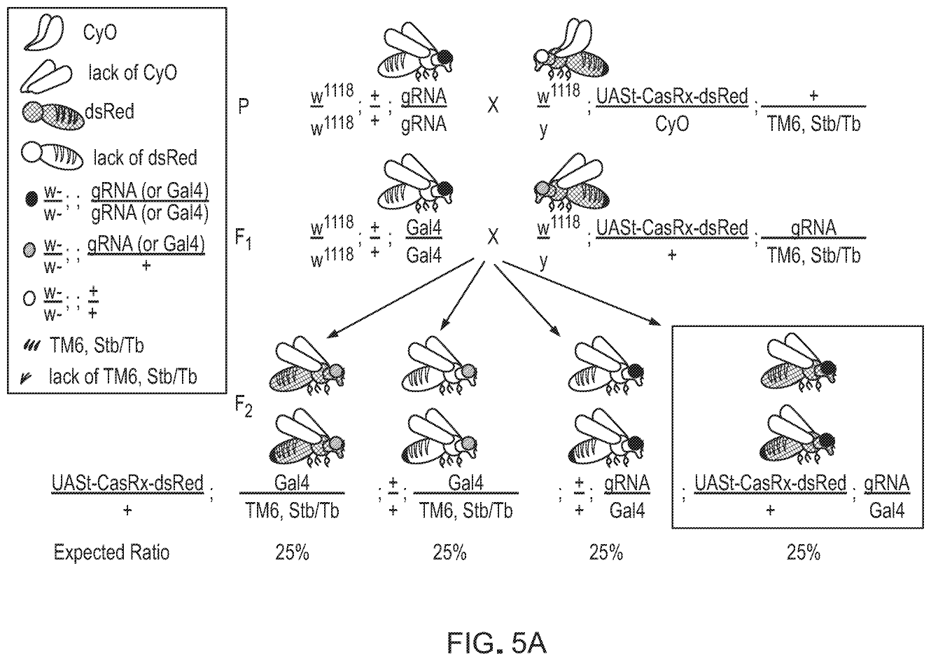

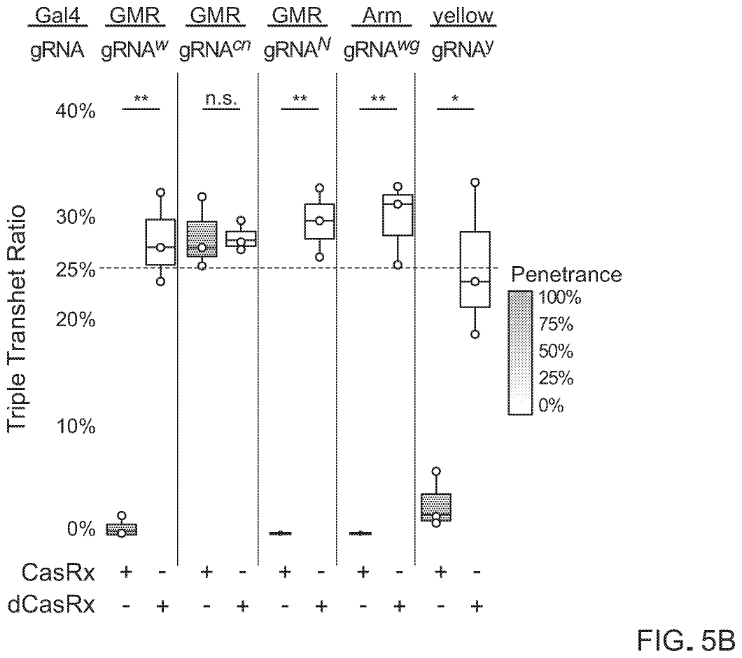

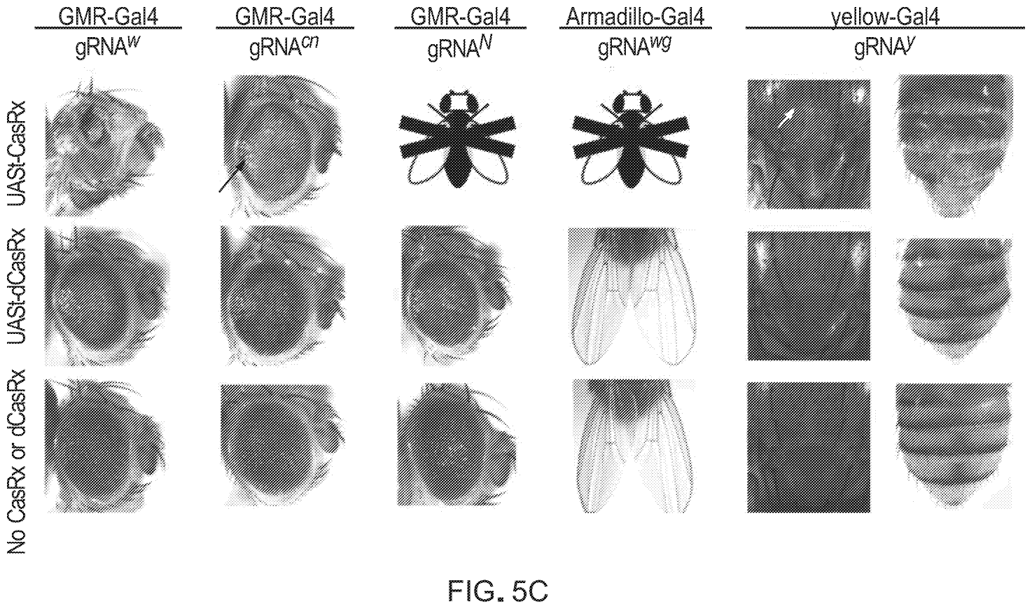

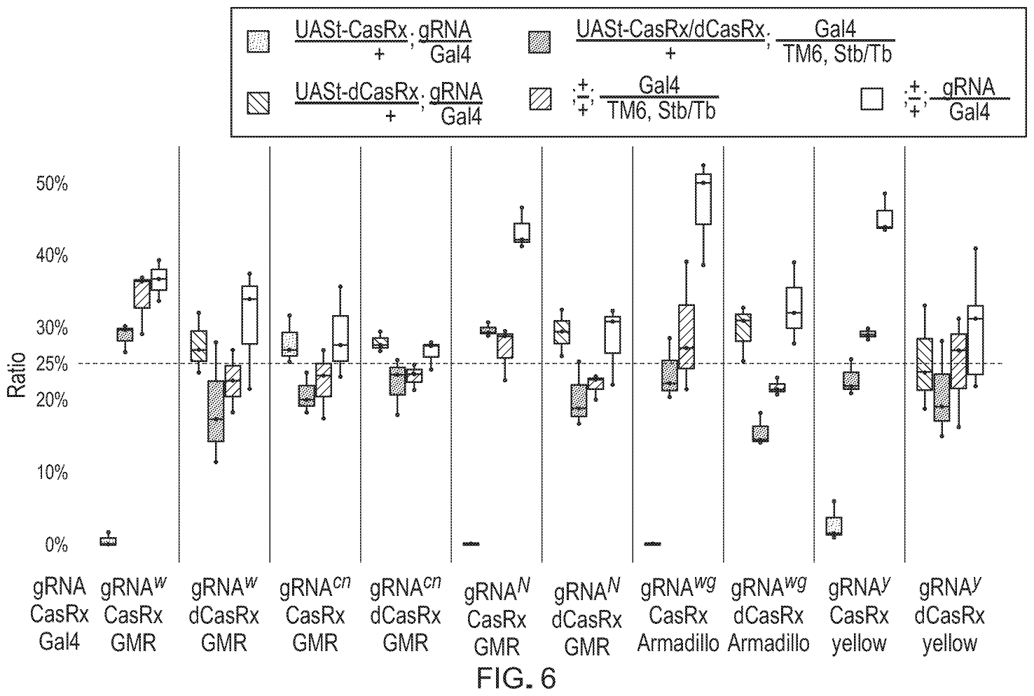

To further explore the utility of programmable RNA-targeting of CasRx in flies, its efficiency was investigated when expression was restricted to specific cell types and tissues by leveraging the classical binary Gal4/UAS system. To develop this system, two transgenes were generated using the UASt promoter to drive expression of either CasRx (UASt-CasRx), or as a negative control dCasRx (UASt-dCasRx) (FIG. 1). These transgenes were integrated site-specifically using a ϕC31 docking site located on the 2nd chromosome (site 8621) and these stocks were homozygous viable (FIG. 1, Table 1). To activate CasRx expression, several available Gal4 driver lines that restricted expression to either the eye (GMR-Gal4), embryos and imaginal discs (armadillo-Gal4), or the wing and body (yellow-Gal4) (Table 1) were used, and the same homozygous gRNA^{array} lines described above targeting w, cn, wg, y, or N (FIG. 1, Table 1) were used. To test this system, a 2-step genetic crossing scheme was performed to generate F₂ triple transheterozygotes (either UASt-CasRx/+; gRNA^{array}/Gal4 or UASt-dCasRx/+; gRNA^{array}/Gal4) (FIG. 2A). This consisted of initially crossing homozygous gRNA^{arrray} (gRNA^{array}/gRNA^{array}) expressing flies to heterozygous, double-balanced UASt-CasRx (UASt-CasRx/Cyo; TM6/+) flies, or the negative control, heterozygous, double-balanced UASt-dCasRx (UASt-dCasRx/Cyo; TM6/+) flies. The second step was to cross the F₁ transheterozygote males expressing both a CasRx ribonuclease and the gRNA^{array} (UASt-CasRx/+; gRNA^{array}/TM6 or UASt-dCasRx/+; gRNA^{array}/TM6) to respective homozygous Gal4 driver lines generating F₂ triple transheterozygotes (UASt-CasRx/+; gRNA^{array}/Gal4 or UASt-dCasRx/+; gRNA^{array}/Gal4) to be scored for phenotypes (FIG. 5A). From these crosses, the results indicated that tissue-specific expression of CasRx can indeed result in expected phenotypes, however this was occasionally accompanied by tissue-specific cell death, or lethality, similar to previous observations described above. For example, from the F₁ cross between gRNA^{w} (UASt-CasRx/+; gRNA^{w}/TM6) and GMR-Gal4 (+/+; GMR-Gal4/GMR-Gal4), of the expected 25% Mendelian inheritance rates survival of only 0.57% viable F₂ triple transheterozygotes (UASt-CasRx/+; gRNA^{w}/GMR-Gal4) was observed, all of which displayed phenotypes in the eye (FIGS. 5B, 5C, and 6, Table 3). The gRNA^{w} F₂ triple transheterozygote inheritance rate was significantly less than the corresponding negative control F₂ triple transheterozygote (UASt-dCasRx/+; gRNA^{w}/GMR-Gal4) inheritance rate which was closer to the expected 25% Mendelian inheritance (27.6%) (FIG. 6, Table 3). Moreover, using the same Gal4 driver (GMR-Gal4) a significant difference in inheritance was also observed for N targeting which resulted in 100% lethality in F₂ triple transheterozygotes (UASt-CasRx/+; gRNA^{N}/GMR-Gal4) compared to 29.3% inheritance rate for the negative control F₂ triple transheterozygotes (UASt-dCasRx/+; gRNA^{N}/GMR-Gal4) (FIGS. 5B, 5C, and 6, Table 3). All gRNA^{cn} F₂ triple transheterozygotes (UASt-CasRx/+, gRNA^{cn}/GMR-Gal4) displayed pigment reduction along with a mild cell death phenotype in their eyes (FIG. 5C, arrows), while sharing comparable inheritance ratios (28% vs 28%) with their negative control F₂ triple transheterozygotes (UASt-dCasRx/+; gRNA^{cn}/GMR-Gal4) (FIG. 6, Table 3). For wg targeting, crosses were performed using the armadillo-Gal4 driver (arm-Gal4) (arm-Gal4/arm-Gal4; +/+) and, interestingly, the F₂ triple transheterozygotes (UASt-CasRx/arm-GAL4; gRNA^{wg}/+) were 100% lethal while the negative control F₂ triple transheterozygotes (UASt-dCasRx/arm-GAL4; gRNA^{wg}/+) were viable and inherited transgenes near the expected rate (29.7%) (FIGS. 5B, 5C, and 6, Table 3). Finally, when targeting y, using the yellow-Gal4 driver (+/+; y-Gal4/y-Gal4) marginal chitin pigment reduction was observed at the back of the thorax and abdomen in F₂ triple transheterozygotes (UASt-CasRx/+; gRNA^{y}/y-Gal4) (FIG. 5C, arrows). Similar to crosses described above, the F₂ triple transheterozygote (UASt-CasRx/+; gRNA^{y}/y-Gal4) inheritance was significantly lower (2.67%) when compared to the control F₂ triple transheterozygote (UASt-dCasRx/+; gRNA^{y}/y-Gal4) inheritance (25.2%), indicating partial lethality during development (FIGS. 5B, 5C, and 6, Table 3). Phenotypes in F₂ triple transheterozygotes (UASt-dCasRx/+; gRNA^{array}/Gal4) was not observed in any of the negative control crosses (FIGS. 5B and 5C, Table 3). Taken together, these results demonstrate that tissue specific expression of CasRx using the classical Gal4/UAS approach can result in expected phenotypes, however, as seen in the ubiquitous binary approach above, cell death phenotypes and lethality were also observed. FIG. 5B shows the inheritance rates of triple transheterozygous flies inheriting 3 transgenes (UASt-CasRx or UASt-dCasRx, gRNA^{array}, and Gal4-driver), corresponding to flies highlighted in red box in panel A. Significant differences in inheritance between CasRx and dCasRx groups were observed in 4 of 5 gene targets with the exception of gRNA^{cn} (gRNA^{w}, p=0.00595; gRNA^{N}, p=0.00402; gRNA^{wg}, p=0.00577; gRNA^{y}, p=0.02205). FIG. 6 shows a plot that includes all genotypes scored in all crosses for UASt-CasRx and UASt-dCasRx. For all 5 gRNA^{array} targets, the inheritance of transheterozygous progeny expressing UASt-CasRx, a Gal4 driver, and a gRNA^{array} were lower compared to the other non-transheterozygous flies and to their corresponding dCasRx control group expressing UASt-dCasRx, a Gal4 driver, and a gRNA^{array}. Table 3 shows the complete data set for the Gal4/UASt-CasRx or Gal4/UASt-dCasRx crosses. Absolute counts of inheritance and phenotype penetrance for the F₂ generation resulting from F₁ transheterozygote males expressing UASt-CasRx/gRNA^{array} or UASt-dCasRx/gRNA^{array} crossed to Gal4 driver lines.

### Example 4: Criteria for CasRx Mediated Phenotypes

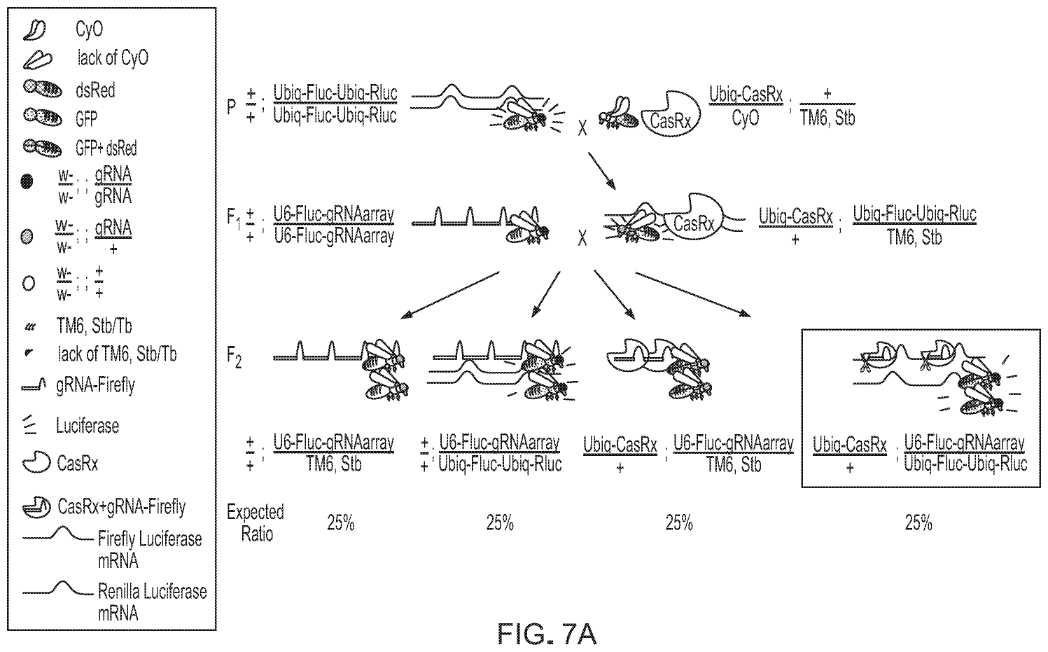

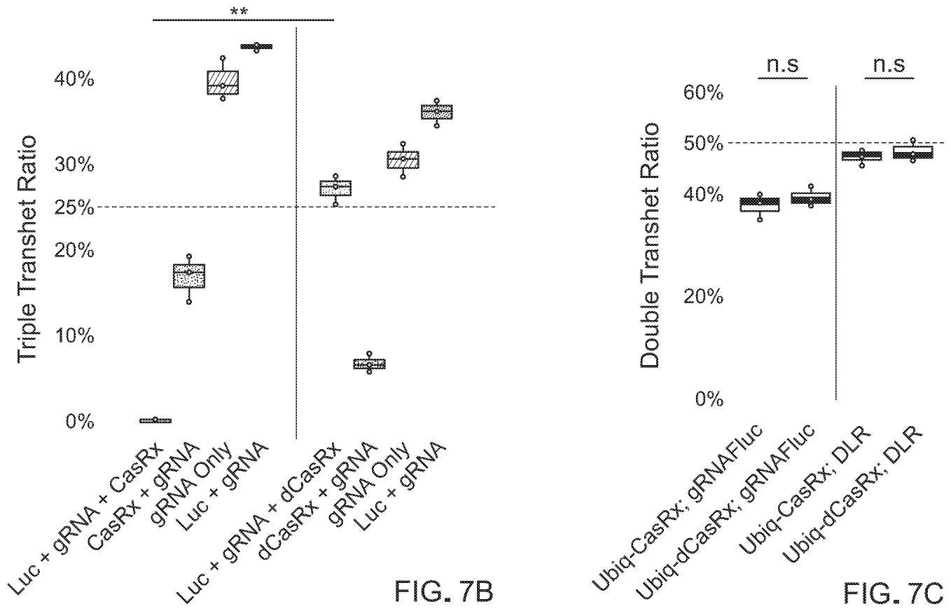

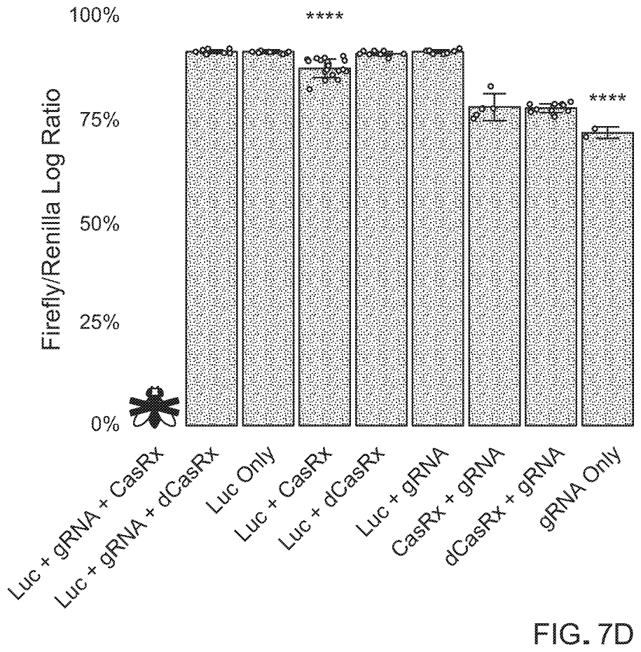

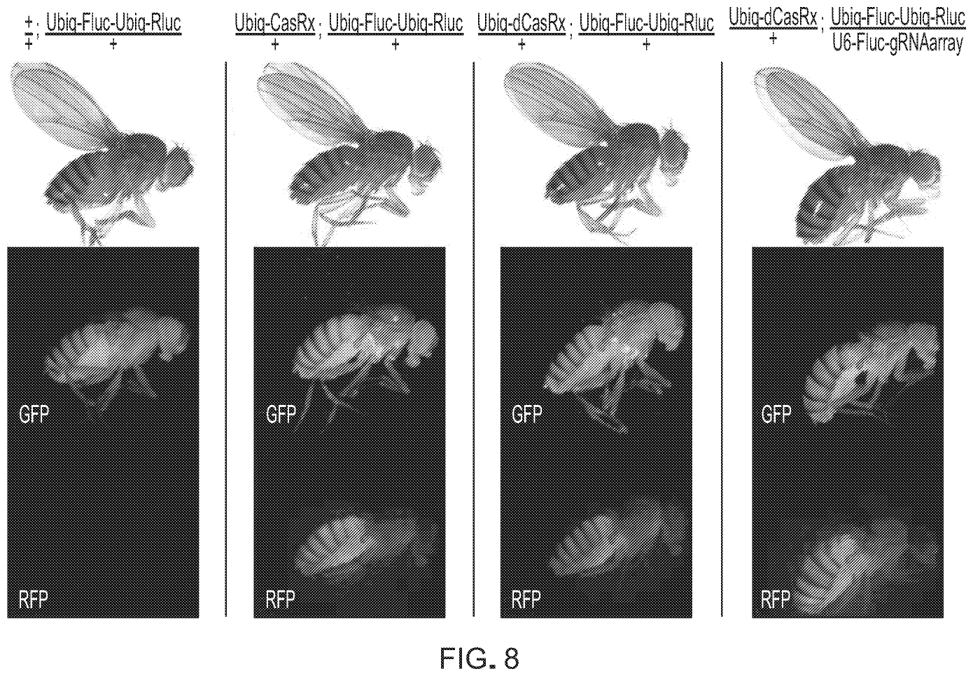

To further explore programmable ribonuclease activity of CasRx and quantify the level of transcript reduction, a dual luciferase reporter assay was developed. This assay comprised of ubiquitously expressed firefly luciferase (Fluc) and a control renilla luciferase (Rluc) (Ubiq-Fluc-Ubiq-Rluc) (FIG. 1) enabling normalization and allowing for quantification of Fluc protein expression reduction resulting from CasRx transcript targeting. The reporter construct was integrated at an available ϕC31 docking site on the 3rd chromosome (site 9744) and generated a homozygous transgenic stock (+/+; Ubiq-Fluc-Ubiq-Rluc/Ubiq-Fluc-Ubiq-Rluc) (FIG. 1, Table 1). A gRNA^{array} targeting Fluc (gRNA^{Fluc}) was then engineered, and a homozygous transgenic stock (+/+; gRNA^{Fluc}/gRNA^{Fluc}) was generated by integrating the gRNA^{array} on the 3rd chromosome using ϕC31 integration (site 8622) (FIG. 1, Table 1). For genetic analysis, a 2-step cross was followed by initially mating heterozygous, double-balanced Ubiq-CasRx (Ubiq-CasRx/CyO; TM6/+) flies, or Ubiq-dCasRx (Ubiq-dCasRx/CyO; TM6/+) negative controls to homozygous dual luciferase reporter flies (Ubiq-Fluc-Ubiq-Rluc/Ubiq-Fluc-Ubiq-Rluc). F₁ transheterozygous males carrying the TM6 balancer chromosome (Ubiq-CasRx/+; Ubiq-Fluc-Ubiq-Rluc/TM6 or Ubiq-dCasRx/+; Ubiq-Fluc-Ubiq-Rluc/TM6) were then crossed to homozygous gRNA^{Fluc} (+/+; gRNA^{Fluc}/gRNA^{Fluc}) expressing flies (FIG. 7A). Interestingly, despite the target gene being non-essential, expressing all three transgenes in F₂ triple transheterozygotes (Ubiq-CasRx/+; gRNA^{Fluc}/Ubiq-Fluc-Ubiq-Rluc) was found to result in 100% lethality compared to control crosses involving Ubiq-dCasRx, where lethality was completely eliminated in the F₂ triple transheterozygotes (Ubiq-dCasRx/+; gRNA^{Fluc}/Ubiq-Fluc-Ubiq-Rluc) (FIG. 7B, FIG. 8). As shown in FIG. 7B, inheritance of all 3 transgenes (Ubiq-CasRx, Ubiq-Fluc-Ubiq-Rluc, and gRNA^{array}) in F₂ progeny was 100% lethal and significantly lower than Ubiq-dCasRx triple transheterozygotes (p=0.001, t-test). FIG. 8 shows dual-luciferase reporter system transgenic markers, with representative markers for each construct. The top row is a bright field image of all respective genotypes involved in the reporter system (a heterozygote is used in the first column to demonstrate the expected GFP expression). w⁺ represents either Ubiq-Fluc-Ubiq-Rluc or gRNA^{Fluc} expression. OpIE2-dsRed expression represents Ubiq-CasRx or Ubiq-dCasRx expression. (Left to right) Ubiq-Fluc-Ubiq-Rluc heterozygote, Ubiq-CasRx/Ubiq-Fluc-Ubiq-Rluc transheterozygote, Ubiq-dCasRx/Ubiq-Fluc-Ubiq-Rluc transheterozygote, and Ubiq-dCasRx/Ubiq-Fluc-Ubiq-Rluc/gRNA^{Fluc} triple transheterozygote. Ubiq-CasRx/Ubiq-Fluc-Ubiq-Rluc/gRNA^{Fluc} triple transheterozygotes were 100% lethal and thus could not be imaged.

Furthermore, it was confirmed that only the combination of all three transgenes (Ubiq-CasRx/+; gRNA^{Fluc}/Ubiq-Fluc-Ubiq-Rluc) resulted in lethality by crossing heterozygous flies expressing Ubiq-CasRx (Ubiq-CasRx/Cyo; +/+) to homozygous flies expressing either gRNA^{Fluc} (+/+; gRNA^{Fluc}/gRNA^{Fluc}) or homozygous flies expressing the dual luciferase reporter transgene (+/+; Ubiq-Fluc-Ubiq-Rluc/Ubiq-Fluc-Ubiq-Rluc). As expected, no distinguishable phenotypes or dramatic influence on inheritance in F₁ transheterozygotes (Ubiq-CasRx/+; gRNA^{Fluc}/+ or Ubiq-CasRx/+; Ubiq-Fluc-Ubiq-Rluc/+) compared to Ubiq-dCasRx controls (Ubiq-dCasRx/+; gRNA^{Fluc}/+ or Ubiq-dCasRx/+; Ubiq-Fluc-Ubiq-Rluc/+) were observed (FIG. 7C, Table 2). As shown in FIG. 7C, inheritance of Ubiq-CasRx/gRNA^{Fluc} or Ubiq-CasRx/Ubiq-Fluc-Ubiq-Rluc did not lead to 100% lethality and inheritance ratios between Ubiq-CasRx and Ubiq-dCasRx transheterozygotes are not significantly different (p=0.41 and p=0.51, respectively, t-test). Next, Fluc and Rluc expression levels in flies of all viable genotypes were measured, and no significant reduction in Fluc expression in the Ubiq-dCasRx triple transheterozygotes (Ubiq-dCasRx/+; gRNA^{Fluc}/Ubiq-Fluc-Ubiq-Rluc) compared to dual luciferase reporter controls was observed, suggesting that Fluc protein expression levels were not reduced by dCasRx targeting (FIG. 7D). However, given the complete embryonic lethality of the Ubiq-CasRx F₂ triple transheterozygotes (Ubiq-CasRx/+; gRNA^{Fluc}/Ubiq-Fluc-Ubiq-Rluc) the luciferase activity in these flies were unable to be measured.

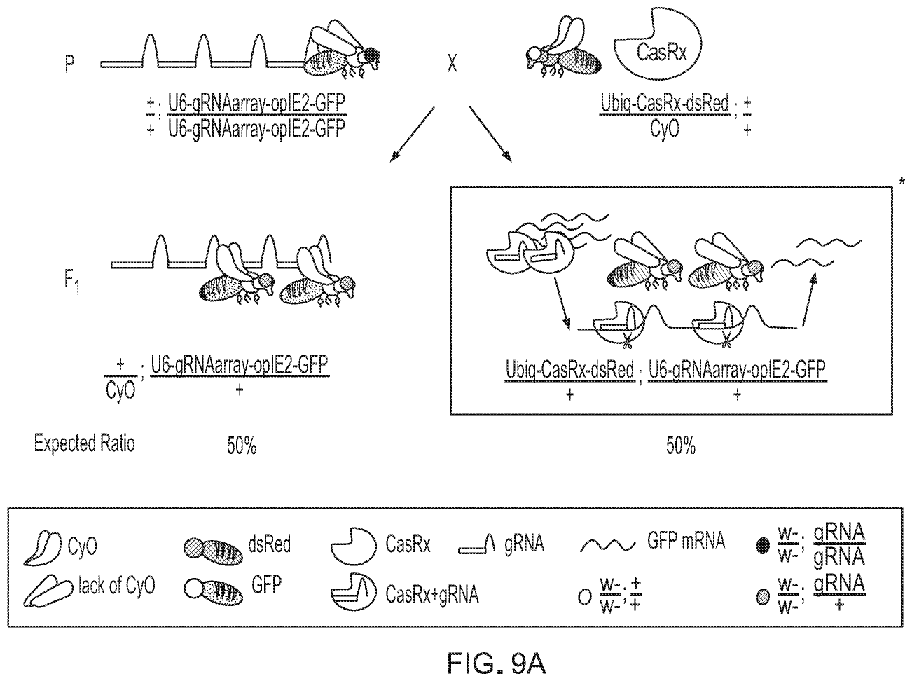

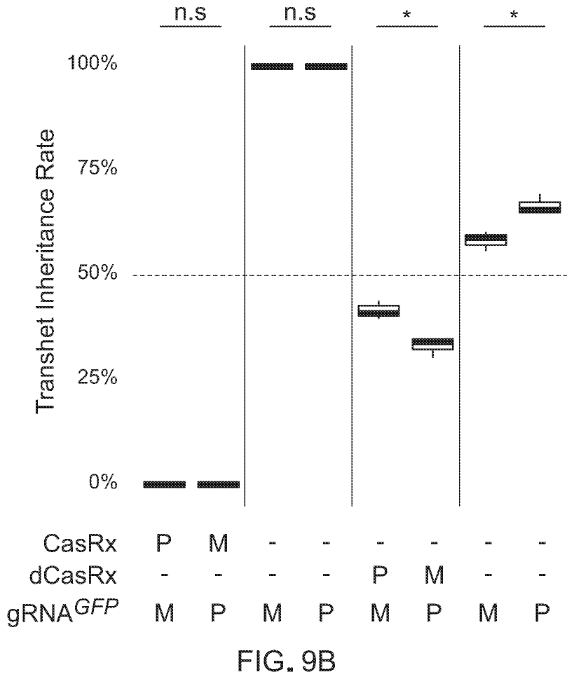

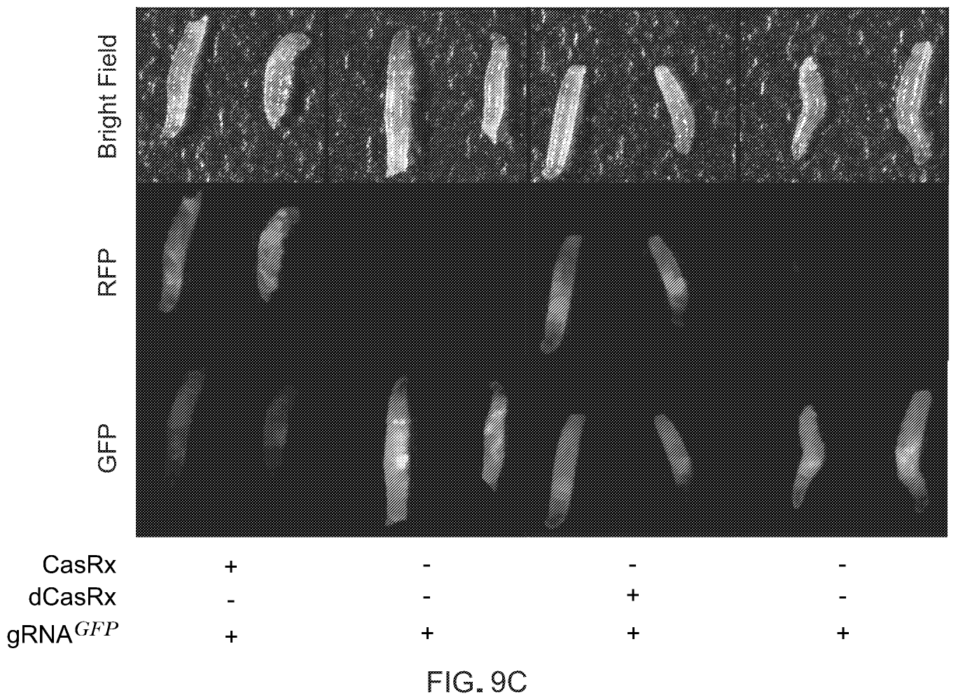

Given the inability to generate and measure luciferase expression from Ubiq-CasRx F₂ triple transheterozygotes (Ubiq-CasRx/+; gRNA^{Fluc}/Ubiq-Fluc-Ubiq-Rluc) in the luciferase crosses described above, a GFP reporter assay was generated to directly visualize CasRx-mediated transcript knockdown. A binary GFP reporter construct was generated, comprised of both a CasRx gRNA^{array} targeting GFP along with GFP expression driven by the broadly expressing OpIE2 promoter (gRNA^{GFP}) (FIGS. 9A-9C, FIG. 1, Table 1). A homozygous transgenic line (+/+; gRNA^{GFP}-OpIE2-GFP/gRNA^{GFP}-OpIE2-GFP) was established by site-specifically integrating the construct at an available ϕC31 docking site located on the 3rd chromosome (site 8622) (FIG. 1, Table 1). To test for GFP transcript targeting, bidirectional crosses was performed between homozygous flies expressing gRNA^{GFP} (+/+; gRNA^{GFP}-OpIE2-GFP/gRNA^{GFP}-OpIE2-GFP) to heterozygous Ubiq-CasRx expressing flies (Ubiq-CasRx/CyO; +/+), or heterozygous Ubiq-dCasRx expressing flies (Ubiq-dCasRx/CyO; +/+) as a negative control (FIG. 9A). Interestingly, 100% adult lethality was observed for F₁ transheterozygotes (Ubiq-CasRx/+; gRNA^{GFP}-OpIE2-GFP/+), while adult lethality was completely eliminated in F₁ transheterozygote controls (Ubiq-dCasRx/+; gRNA^{GFP}-OpIE2-GFP/+) and lethality was observed regardless of maternal or paternal deposition of CasRx (FIG. 9B, Table 2). Given that GFP expression was also visible in larvae, the development of the F₁ progeny was monitored and it was observed that Ubiq-CasRx transheterozygotes survived only to the first instar developmental stage, but not beyond (FIG. 4B). Given this survival, first instar transheterozygote (Ubiq-CasRx/+; gRNA^{GFP}-OpIE2-GFP/+) larvae was imaged and complete reduction in GFP expression for Ubiq-CasRx transheterozygote larvae as compared to Ubiq-dCasRx transheterozygote (Ubiq-dCasRx/+; gRNA^{GFP}-OpIE2-GFP/+) control larvae was observed (FIG. 9C). Taken together, these results strongly indicate that CasRx possesses programmable RNA-targeting activity and the lethality is dependent upon the availability of a broadly expressed target sequence as well as enzymatic RNA cleavage mediated by the positively charged residues of CasRx HEPN domains.

### Example 5: Quantification of CasRx Mediated On/Off Target Activity

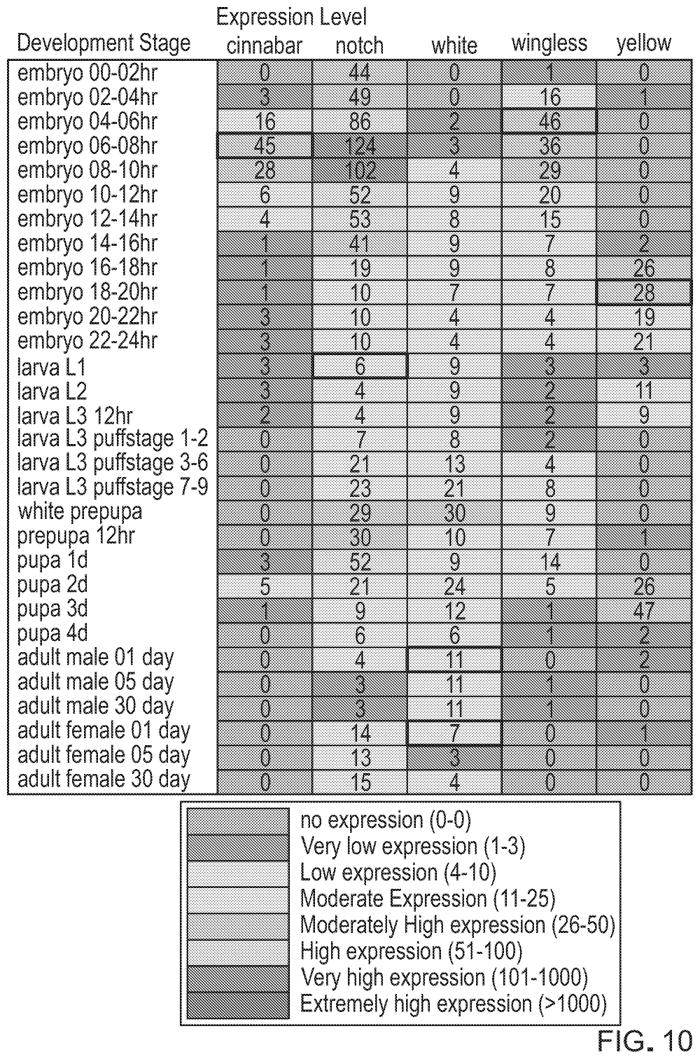

Upon obtaining distinct visual phenotypes from Ubiq-CasRx transheterozygotes (Ubiq-CasRx/+; gRNAa^{array}/+), both the on- and potential off-target transcript reduction rates were quantified. All gRNA^{array} target genes from our binary crosses producing either highly-penetrant, visible phenotypes (w, cn, and wg) or lethal phenotypes (N, y, and GFP) were analyzed (Table 5). To do so, whole-transcriptome RNAseq analysis was implemented comparing F₁ Ubiq-CasRx transheterozygotes (Ubiq-CasRx/+; gRNAa^{array}/+) compared to control F₁ Ubiq-dCasRx transheterozygotes (Ubiq-dCasRx/+; gRNA^{array}/+) (FIG. 2A box with asterisk, FIG. 9A box with asterisk, Table 5). Using available transcriptome data of Drosophila melanogaster (modENCODE), total RNA was extracted at various stages of development when high transcript expression levels were expected for each target gene with the exception of GFP, where first instar larvae was sequenced (FIG. 10, Table 5). FIG. 10 shows modENCODE transcript expression relative to Drosophila melanogaster development. Black box indicates which developmental period was chosen for RNA sequencing of samples for analysis of CasRx-mediated transcript knockdown in Ubiq-CasRx vs Ubiq-dCasRx comparison. Not included: GFP 1^{st} instar larvae were chosen for analysis of GFP transcript knockdown.

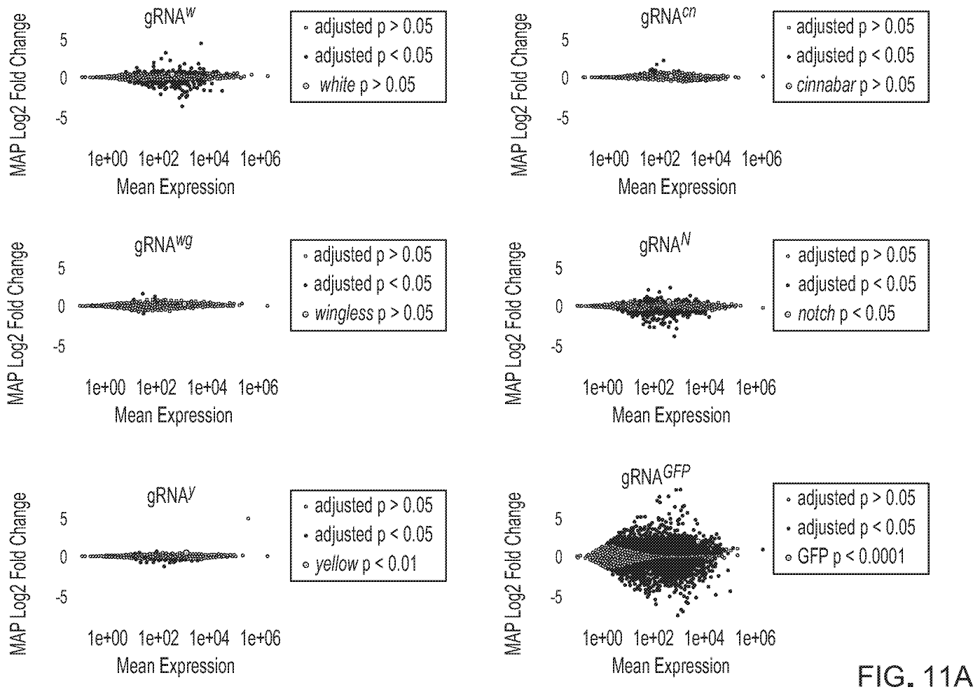

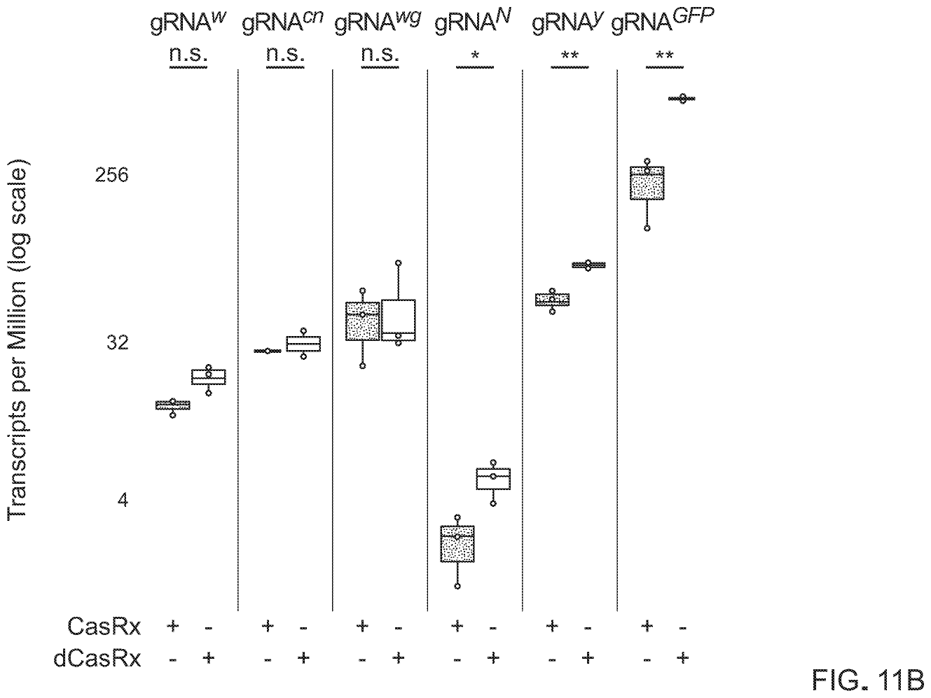

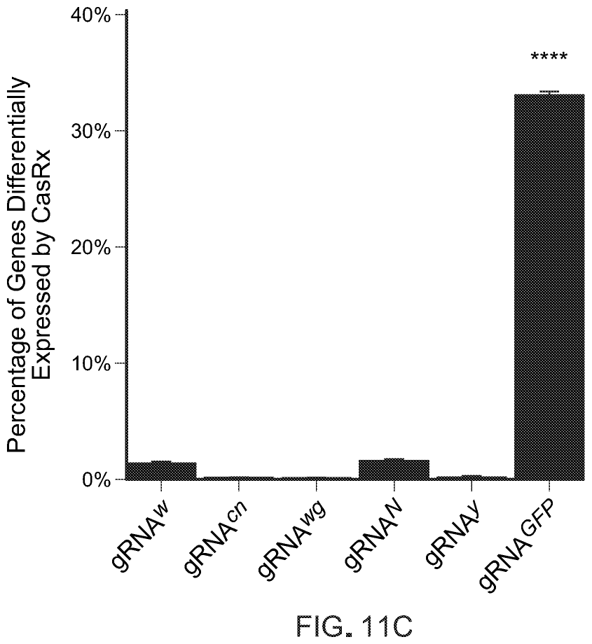

In total 34 samples were analyzed (Table 5), and CasRx was found to be capable of consistent on-target transcript reduction based on bioinformatic analysis (FIGS. 11A and 11B). For example, of the 6 target genes CasRx was found to be able to target and significantly reduce the target transcript expression level of 3 genes compared to dCasRx controls: N, y, and GFP (FIG. 11B, Table 6-Table 11). Although significant transcript reduction targeting w, cn, or wg was not observed, relative expression reduction was consistently observed comparing Ubiq-CasRx samples to Ubiq-dCasRx controls indicating some degree of on-target reduction (FIG. 11B, Table 6-Table 8, Table 12-Table 14). The number of genes with significantly misexpressed transcripts were also quantified comparing Ubiq-CasRx to Ubiq-dCasRx using DESeq2 (FIG. 11A, red dots). FIGS. 11A-11C show quantification of CasRx-mediated on/off target activity. FIG. 11A shows maximum a posteriori (MAP) estimates for the logarithmic fold change (LFC) of transcripts. DESeq2 pipeline was used for estimating shrunken MAP LFCs. Wald test with Benjamini-Hochberg correction was used for statistical inference. Grey dots represent transcripts not significantly differentially expressed between Ubiq-CasRx and Ubiq-dCasRx group (p>0.05). Red dots represent transcripts significantly differentially expressed between CasRx and dCasRx group (p<0.05). Pink dot identifies the respective CasRx target gene for each analysis (p value indicated in the inset). FIG. 11B shows transcript expression levels (TPM) of transcripts targeted with CasRx or dCasRx. Student's t-test was used to calculate significance (P values: w=0.07, cn=0.65, wg=0.73, N=0.04, y=0.006, GFP=0.008). FIG. 11C shows percentage of transcripts significantly differentially expressed resulting from CasRx cleavage. Pairwise two-sample test for independent proportions with Benjamini-Hochberg correction was used to calculate significance. Table 5 shows Illumina RNA sequencing whole-transcriptome analysis samples. List of samples, in triplicate, analyzed for quantification of CasRx-mediated transcript knockdown in comparison to dCasRx. The genotype, development stage or tissue type, and corresponding vectors are elaborated (Experimental=Ubiq-CasRx, Control=Ubiq-dCasRx).

These results demonstrate the use of CasRx for programmable RNA-targeting in flies. Although cellular toxicity from ubiquitous expression of CasRx and dCasRx was observed, as well as unexpected lethality and tissue necrosis in both bidirectional and Gal4/UAS crosses, clear, visible phenotypes as well as quantitative evidence demonstrating on-target transcript cleavage were obtained. This is the first demonstration of a programmable RNA targeting Cas system in Drosophila melanogaster, paving the way to providing an alternative approach for gene knockdown studies in vivo, however with further optimization may be required to increase the CasRx on-target cleavage rates.

Through analysis of RNaseq data, consistent reduction in target gene expression was found, however only 50% of the samples crossed a significance threshold. Since clear phenotypes were observed indicating on-target transcript knockdown for w, cn, and wg targeting, but no significant on-target reduction was found through DESeq2 analysis, it is hypothesized that developmental timing of sample collection is imperative for quantifying transcript knockdown efficiency. Notwithstanding, significant on-target transcript expression reduction were obtained that also corresponded with lethality phenotypes (y, N, and GFP) and resulted in numerous misexpressed genes. Targeting GFP, a non-essential gene, produced the largest quantity of misexpressed genes as well as the most significant fold change compared to all other gene targets analyzed. Interestingly, Gadd45, a gene involved in cellular arrest and apoptosis in Drosophila melanogaster, was found to be significantly misexpressed in 4 samples (w, N, y, and GFP). It is possible that CasRx cleavage may result in a dramatically higher number of misexpressed genes and possible lethality or cellular apoptosis.

Evidence of off-target effects resulting from catalytic activity of CasRx identified through DESeq2 analysis is provided. This is the first report of off-target activity occurring from the application of a Cas13 ribonuclease in eukaryotic cells, and key factors that determine lethality are highlighted. Two main factors contributing to CasRx-mediated lethality were identified: the catalytic activity of the CasRx HEPN domains and the presence of the target transcript resulting in on-target cleavage. For example, lethality and tissue necrosis phenotypes were eliminated comparing dCasRx to CasRx crosses and no lethality was observed when crossing Ubiq-CasRx expressing flies to gRNA^{Fluc} expressing flies in the absence of the Fluc transcript. These results recapitulate previous mechanistic analysis of CasRx and other Cas13 ribonucleases demonstrating that off-target activity following targeted transcript cleavage is a native feature of Cas13 ribonuclease applications.

Cas13 enzymes have been proposed to be highly specific ribonucleases with the ability to replace previously developed RNAi technologies. dCas13 enzymes retain efficient RNA binding activity and can be modified to effectively diminish the promiscuous RNase activity of Cas13 ribonucleases. Previous studies have utilized dCas13 enzymes for RNA base editing, dynamic imaging of RNA, and to manipulate pre-mRNA splicing, demonstrating both the specificity and versatility of dCas13 RNA binding. Further modifications to dCasRx may provide viable alternatives for targeted transcript degradation in flies through manipulation of the nonsense mediated mRNA decay (NMD) pathway or through inhibition of proper transcript splicing. However, there remain advantages to the catalytic activity of CasRx and other Cas13 ribonucleases, including the promiscuous RNase activity these enzymes exhibit.

Due to the programmable nature of CRISPR systems, numerous arthropods can theoretically be transgenically engineered and studied applying CasRx. This report provides a preliminary characterization of CasRx function in arthropods and opens up numerous avenues to explore transcript targeting, virus targeting, and technological development of RNA binding applications. One potential application could involve controlling the spread of vector-borne illnesses in arthropods, such as mosquitoes. Recently, in cell culture experiments, a Cas13 ribonuclease was used to directly target a variety of ssRNA viruses known to infect humans. Aedes mosquitoes are primary vectors for ssRNA viruses such as dengue virus, with an estimated 390 million people infected annually. ssRNA viruses transmitted through Aedes mosquitoes rapidly evolve in both vectors and humans, which presents a significant challenge for generating efficient vaccines or biological methodologies for reducing transmission. The CasRx RNA targeting system in arthropods provides a platform to reduce the spread of ssRNA arboviruses by directly targeting ssRNA virus genomes in a programmable manner. In this case, collateral cleavage and tissue-specific cell death may serve as a significant advantage for ssRNA virus targeting in arbovirus vectors.

### Example 6: Methods Used in the Above Experiments

### Design and Assembly of Constructs

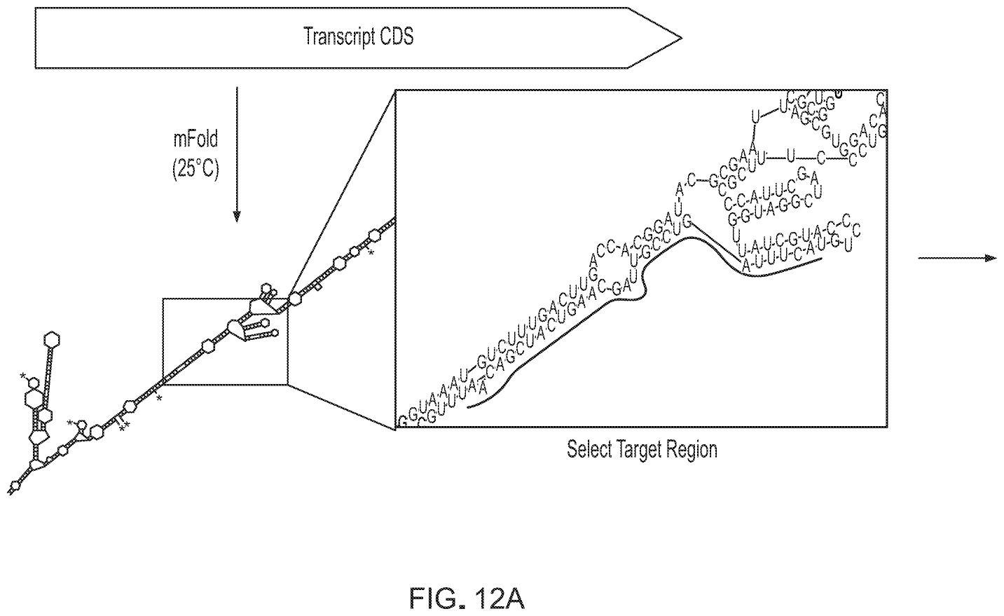

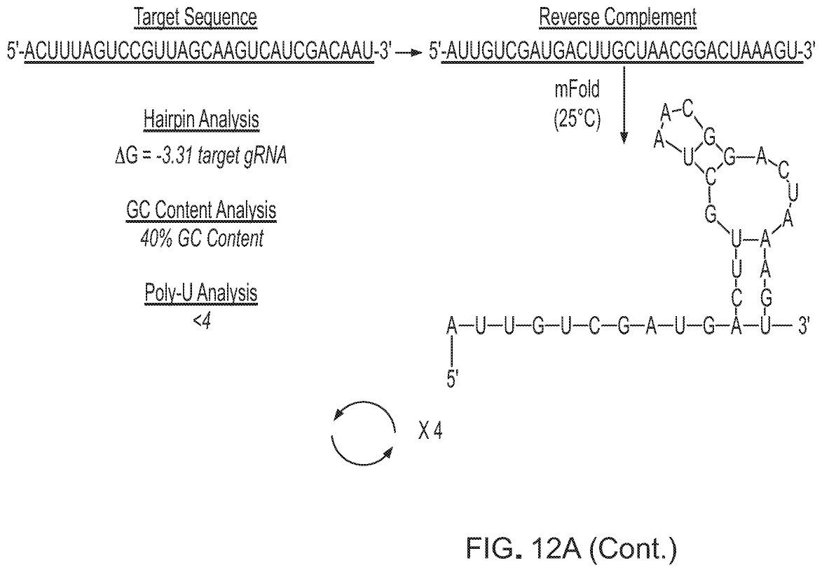

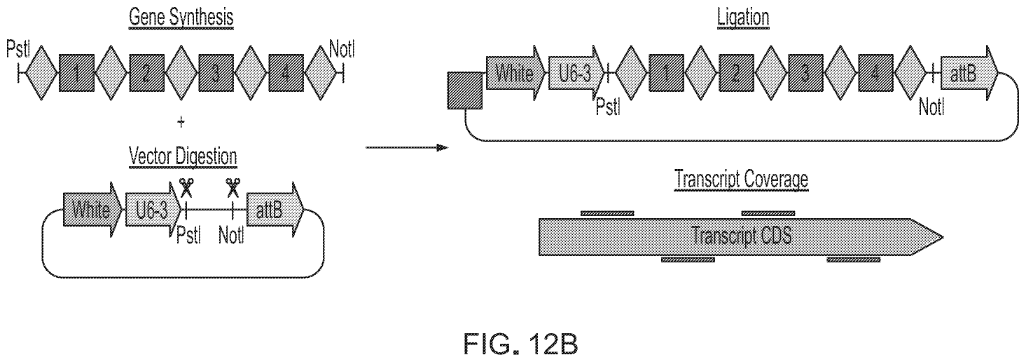

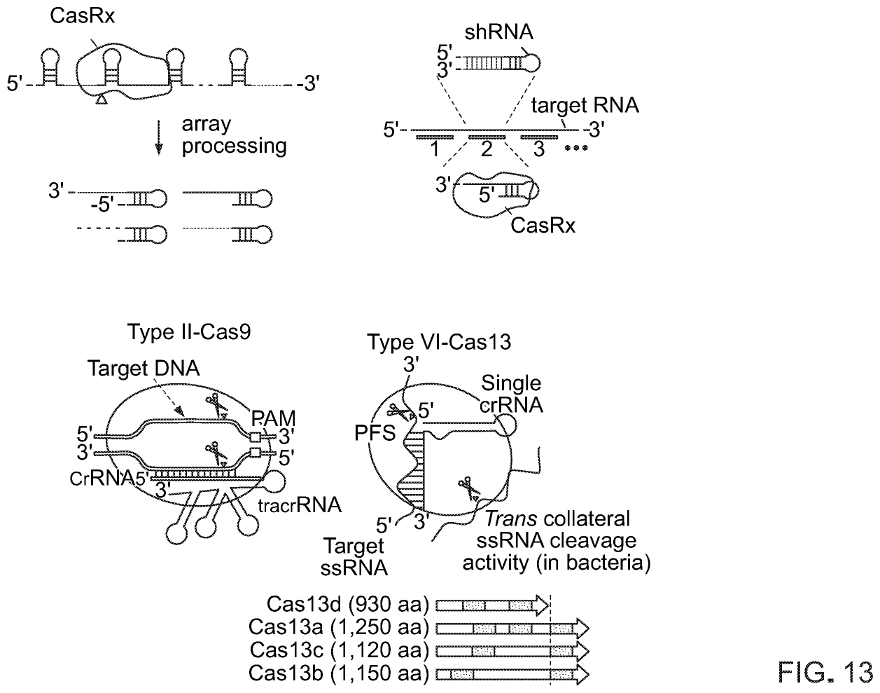

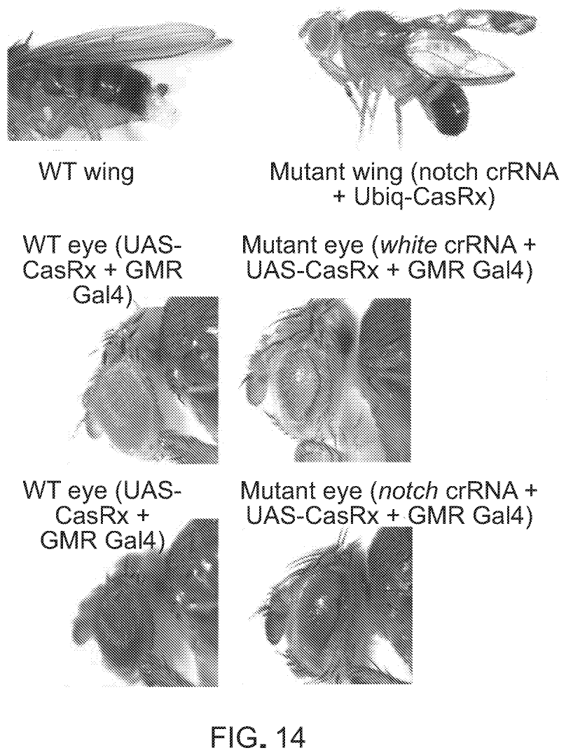

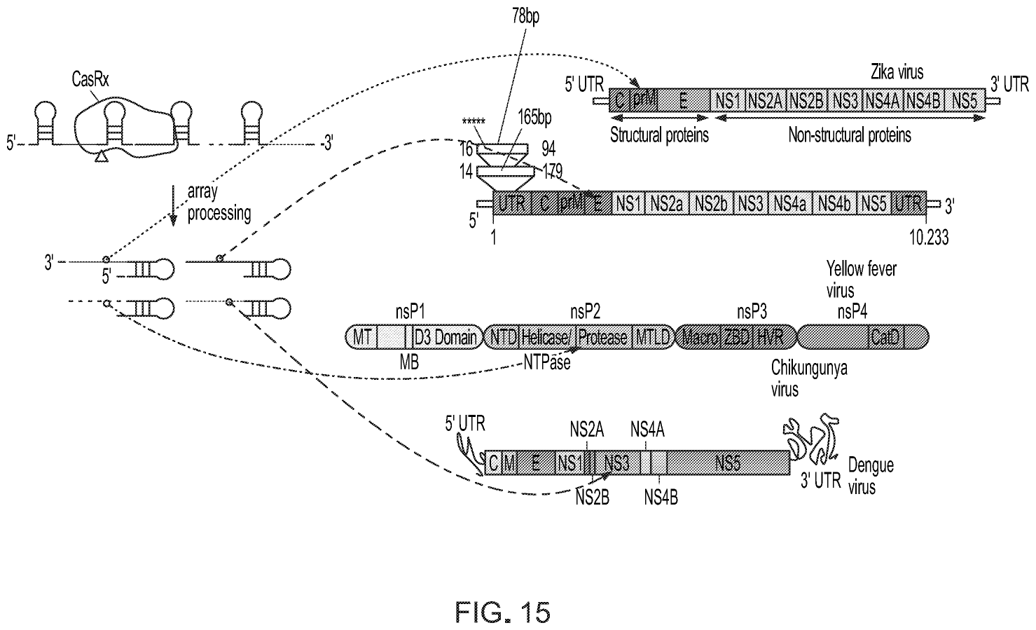

To select RNA sites for CasRx targeting, target genes were analyzed to identify 30-nucleotide regions that had no poly-U stretches greater than 4 bp, had GC base content between 30% and 70%, and were not predicted to form very strong hairpin structures. Care was also taken to select target sites in RNA regions that were predicted to be accessible, such as regions without predicted RNA secondary or tertiary structure (FIGS. 12A and 12B). FIGS. 12A and 12B show CasRx-gRNA^{array} transcript target selection and construct generation. FIG. 12A is a schematic representing the workflow for gRNA choice. The transcript CDS for a GOI is entered into the mFold database (condition: 25° C.) where predictive analysis identifies the most probable secondary and tertiary folding of the entire transcript. We then chose specific regions predicted to be easily accessible for CasRx targeting (blue line), contains GC content between 30% and 70%, and possesses no poly-U stretches longer than 4 nt. We then convert the target sequence into the reverse complement (red line) and enter this spacer sequence into mFold (condition: 25° C.) for hairpin analysis. This is repeated until 4 optimal target sites are selected. FIG. 12B is a schematic showing the generation of gRNA^{array} construct. dsDNA is first synthesized to contain 4 spacer and 5 DR sequences with specific restriction sites present on the 5′ and 3′ end of the DNA. Simultaneously the vector backbone containing the miniwhite marker, a U6:3 promoter fragment, and an attB site is digested using the corresponding restriction sites of the dsDNA gene fragment. The two pieces are then ligated together to generate a CasRx gRNA^{array} covering the majority of the transcript for the GOI. All RNA folding/hairpin analysis was performed using the mFold server. For transgenic gRNA arrays, 4 targets per gene were selected to ensure efficient targeting. Previously, Cas13d ribonucleases were shown to possess gRNA processing RNase activity without additional helper ribonucleases.

Four CasRx- and dCasRx-expressing constructs were assembled under the control of one of two promoters: Ubiquitin-63E (Ubiq) or UASt (Ubiq-CasRx, Ubiq-dCasRx, UASt-CasRx, UASt-dCasRx) using the Gibson enzymatic assembly method. A base vector (Addgene plasmid #112686) containing piggyBac and an attB-docking site, Ubiq promoter fragment, SpCas9-T2A-GFP, and the Opie2-dsRed transformation marker was used as a template to build all four constructs. To assemble constructs OA-1050E (Addgene plasmid #132416, Ubiq-CasRx) and OA-1050R (Addgene plasmid #132417, Ubiq-dCasRx), the SpCas9-T2A-GFP fragment was removed from the base vector by cutting with restriction enzymes SwaI and PacI, and then replaced with CasRx and dCasRx fragments amplified with primers 1050E.C3 and 1050E.C4 (Table 15) from constructs pNLS-RfxCas13d-NLS-HA (pCasRx) and pNLS-dRfxCas13d-NLS-HA (pdCasRx), respectively. To assemble constructs OA-1050L (Addgene plasmid #132418, UASt-CasRx) and OA-1050S (Addgene plasmid #132419, UASt-dCasRx), the base vector described above was digested with restriction enzymes NotI and PacI to remove the Ubiq promoter and SpCas9-T2A-GFP fragments. And then UASt promoter fragment and CasRx or dCasRx fragments, respectively, were cloned in. The UASt promoter fragment was amplified from plasmid pJFRC81, with primers 1041.C9 and 1041.C11 (Table 15). The CasRx and dCasRx fragments were amplified with primers 1050L.C1 and 1050E.C4 (Table 15) from constructs pCasRx and pdCasRx, respectively.

Seven four-gRNA-array constructs were designed, OA-1050G (Addgene plasmid #132420), OA-1050I (Addgene plasmid #132421), OA-1050J (Addgene plasmid #133304), OA-1050K (Addgene plasmid #132422), OA-1050U (Addgene plasmid #132423), OA-1050V (Addgene plasmid #132424), OA-1050Z4 (Addgene plasmid #132425), targeting transcripts of white, Notch, GFP, firefly luciferase, cinnabar, wingless, and yellow, respectively. To generate a base plasmid, OA-1043, which was used to build all the final seven four-gRNA-array constructs, Addgene plasmid #112688 containing the miniwhite gene as a marker, an attB-docking site, a D. melanogaster polymerase-3 U6 (U6:3) promoter fragment, and a guide RNA fragment with a target, scaffold, and terminator sequence (gRNA) was digested with restriction enzymes AscI and XbaI to remove the U6:3 promoter and gRNA fragments. Then the U6:3 promoter fragment amplified from the same Addgene plasmid #112688 with primers 1043.C1 and 1043.C23 (Table S16), was cloned back using Gibson enzymatic assembly method. To generate constructs OA-1050G, OA-1050I, OA-1050K, OA-1050U, OA-1050V, OA-1050Z4, plasmid OA-1043 was digested with restriction enzymes PstI and NotI, a fragment containing arrays of four tandem variable gRNAs (comprised of a 36-nt direct repeat (DR) and a 30-nt spacer) corresponding to different target genes respectively, followed by an extra DR and a 7 thymines terminator was synthesized and subcloned into the digested backbone using Gene Synthesis (GenScript USA Inc., Piscataway, N.J.). To generate constructs OA-1050J, a fragment containing arrays of four tandem variable gRNAs targeting GFP with an extra DR and a 7 thymines terminator, followed by the OpIE2-GFP marker was synthesized and subcloned into the above digested OA-1043 backbone using Gene Synthesis (GenScript USA Inc., Piscataway, N.J.).

To assemble construct OA-1052B (Addgene plasmid #132426), the dual-luciferase expression vector consisted of firefly luciferase linked with T2A-EGFP (Fluc-T2A-EGFP) and renilla luciferase both driven by Ubiq promoter fragment (Ubiq-Fluc-T2A-eGFP-Ubiq-Rluc), Addgene plasmid #112688 containing the white gene as a marker, an attB-docking site as described previously was digested with enzymes AscI and XbaI, and the following components were cloned in using the Gibson enzymatic assembly method: i) a D. melanogaster Ubiq promoter fragment amplified from Addgene plasmid #112686 with primers 1052B.C1 and 1052B.C2; ii) a custom gBlocks® Gene Fragment (Integrated DNA Technologies, Coralville, Iowa) of a firefly luciferase coding sequence; iii) a T2A-eGFP fragment amplified from Addgene plasmid #112686 with primers 908.A1 and 908.A2; iv) a custom gBlocks® Gene Fragment containing a p10 3′UTR fragment, reversed renilla luciferase followed by an SV40 3′UTR fragment; v) another Ubiq promoter fragment as reversed sequence amplified from Addgene plasmid #112686 with primers 908.A3 and 908.A4 (Table 15). All plasmids and sequence maps were made available for download and/or order at Addgene (www.addgene.com) with identification numbers listed in FIG. 1 and Table 1. Table 15 shows primers used for vector construction. A list of primers and their respective sequences used to generate the constructs used in this study.

### Fly Genetics and Imaging

Flies were maintained under standard conditions at 26° C. Embryo injections were performed at Rainbow Transgenic Flies, Inc. (http://www.rainbowgene.com). All CasRx and dCasRx expressing lines were generated by site-specifically integrating constructs at available ϕC31 integration sites on the 2nd chromosome (site 8621 (UAS/-(d)CasRx) and attp40w (Ubiq-(d)CasRx)). Homozygous lines were established for UASt-CasRx and UASt-dCasRx and heterozygous balanced lines were established for Ubiq-CasRx and Ubiq-dCasRx (over Curly of Oster: CyO). All gRNA^{array} expressing lines were generated by site-specifically integrating constructs at an available ϕC31 integration site on the 3rd chromosome (site 8622). Homozygous lines were established for all gRNA^{array} expressing flies. Dual-luciferase reporter expressing lines were generated by site-specifically integrating the constructs at an available ϕC31 integration site on the 3rd chromosome (site 9744). Homozygous lines were established for the dual-luciferase reporter expressing flies.

To genetically assess efficiency of CasRx ribonuclease activity, at 26° C., Ubiq-CasRx and Ubiq-dCasRx expressing lines were bidirectionally crossed to gRNA^{array} expressing lines and let lay for 4 days before removing parents. F1 transheterozygotes were scored for inheritance and penetrance of observable phenotypes up to 17 days post initial laying (13-17 days). Embryo, larvae, and pupae counts preceded by crossing male Ubiq-CasRx and Ubiq-dCasRx expressing flies to female gRNA^{array} expressing flies. Flies were incubated at 26° C. for 48 h with yeast to induce embryo laying. Flies were then transferred to embryo collection chambers containing yeast-smeared grape-juice plates and incubated at 26° C. overnight (16 h). The grape-juice plates were then removed, embryos counted, and the grape-juice plates incubated for 24 h at 26° C. Total larvae and transheterozygote larvae were then counted and the grape-juice plates transferred to jars and incubated at 26° C. Once all larvae reached the pupal stage, total and transhet pupae were counted. Finally, total adult flies and total adult transheterozygotes were counted 20 days post initial lay. Each genetic cross was set using 5♂ and 10♀ (paternal CasRx) or 4♂ and 8♀ (maternal CasRx) flies in triplicate.

To investigate the tissue-specific activity of CasRx, a 2-step crossing scheme was designed to generate F₂ triple transheterozygotes (FIG. 3A). First, double balanced UASt-CasRx or UASt-dCasRx expressing flies (♂) were crossed to homozygous gRNA^{array} expressing flies (♀) to generate F₁ transheterozygote males carrying TM6 balancer chromosome. The F₁ transheterozygote males carrying TM6 were then crossed with a Gal4 driver expressing line. Marked by the presence of dsRed, the UASt-CasRx or UASt-dCasRx marker, red eyes, and the lack of TM6, F₂ triple transheterozygotes inheritance and phenotype penetrance was scored. Each cross was set using 1♂ and 10♀ flies in triplicate. Following a similar 2-step cross, the efficiency of CasRx mediated transcript reduction at the protein level was investigated by utilizing a dual luciferase reporter assay (FIG. 7A). Double balanced Ubiq-CasRx or Ubiq-dCasRx expressing flies were initially crossed to luciferase reporter expressing flies. F₁ transheterozygote males carrying TM6 were selected and crossed to homozygous gRNA^{Fluc} expressing flies. Selecting for the Ubiq-CasRx or Ubiq-dCasRx marker, dsRed, red eyes, and against TM6, F₂ triple transheterozygotes inheritance was scored and males were frozen at −80° C. prior to luciferase analysis. Each cross was set using 1♂ and 10♀ flies in triplicate. Flies were imaged on the Leica M165FC fluorescent stereomicroscope equipped with a Leica DMC4500 color camera. Image stacks of adult flies were taken in Leica Application Suite X (LAS X) and compiled in Helicon Focus 7. Stacked images were then cropped and edited in Adobe Photoshop CC 2018.

### Illumina RNA-Sequencing

Total RNA was extracted from F₁ transheterozygous flies at different developmental stages based on the reported highest expression level available through modENCODE analysis (FIG. 10). gRNA^{w}: transheterozygous adult heads were cut off one day after emerging and frozen at −80° C. gRNA^{cn}, gRNA^{wg}, gRNA^{y}: flies were incubated in vials for 48 h with yeast to induce embryo laying. Flies were then transferred to embryo collection chambers containing yeast-smeared grape-juice plates and incubated at 26° C. for 3 h. Flies were then removed and embryos on grape-juice plates incubated for additional time related to target gene (gRNA^{wg}=3 h, 3-6 h total; gRNA^{cn}=5 h, 5-8 h total; gRNA^{y}=17 h, 17-20 h total). Embryos were removed from grape-juice plates, washed with diH2O, and frozen at −80° C. gRNA^{N}, gRNA^{GFP}: flies were incubated in vials for 48 h with yeast to induce embryo laying. Flies were then transferred to a new vial and allowed to lay overnight (16 h). Adults were removed and the vials were incubated at 26° C. for 24 h. Transheterozygote first instar larvae were then picked (based on dsRed expression) and frozen at −80° C.

For all samples, total RNA was extracted using Qiagen RNeasy Mini Kit (Qiagen 74104). Following extraction, total RNA was treated with Invitrogen Turbo™ DNase (Invitrogen AM2238). RNA concentration was analyzed using Nanodrop One^{C} UV-vis spectrophotometer (ThermoFisher ND-ONEC-W). RNA integrity was assessed using RNA 6000 Pico Kit for Bioanalyzer (Agilent Technologies #5067-1513). RNA-seq libraries were constructed using NEBNext Ultra II RNA Library Prep Kit for Illumina (NEB #E7770) following the manufacturer's instructions previously. three replicates for all CasRx and dCasRx samples were sequenced and analyzed with the exception of gRNA^{cn} where 2 replicates were analyzed. In total 34 samples, 17 CasRx experimental samples and 17 dCasRx control samples, were sequenced and analyzed.

### Bioinformatics

To further understand CasRx-induced differential gene expression profiles, the raw transcript counts were normalized by transcripts per million (TPM) and maximum a posteriori (MAP) method was used with the original shrinkage estimator in DESeq2 pipeline to estimate transcript logarithmic fold change (LFC) (47). Wald test with Benjamini-Hochberg correction was used for statistical inference. The detailed analysis results are presented in Tables 7-12. Per DESeq2 analysis requirement, some values are shown as NA due to the following reasons: 1) if all samples for a given transcripts have 0 transcript counts, this transcript's baseMean will be 0 and its LFC, p value, and padj will be set to NA; 2) If one replicate of a transcript is an outlier with extreme count (detected by Cook's distance), this transcript's p value and padj will be set to NA. 3) If a transcript is found to have a low mean normalized count after automatic independent filtering, this transcript's padj will be set to NA.

### Luciferase Assays

To measure the efficacy of targeted CasRx knockdown a dual Luciferase reporter system comprised of both Firefly and Renilla Luciferase was utilized. A 2-step genetic crossing scheme was performed (FIG. 7A), and F₂ male triple transheterozygotes were collected for luciferase quantification. Flies were aged between 2-4 days at 26° C. then frozen at −80° C. Each assay was performed on 5 male flies and 5 μl of lysed tissue was used to measure Luciferase activity. Luciferase activity in flies was then analyzed using a Dual-Luciferase® Reporter Assay System with a Glomax 20/20 Luminometer (Promega E1910 & E5331).

### ADDITIONAL EMBODIMENTS

Embodiment 1: A method of modifying a target locus of interest in vivo in an organism, comprising delivering to said locus a Type VI CRISPR-Cas loci effector protein and one or more nucleic acid components, wherein at least the one or more nucleic acid components is engineered and the effector protein forms a complex with the one or more nucleic acid components and upon binding of said complex to the target locus of interest the effector protein induces a modification of the target locus of interest.
Embodiment 2: The method of Embodiment 1, wherein the target locus of interest comprises RNA.
Embodiment 3: The method of Embodiment 2, wherein the target locus of interest comprises endogenous mRNA.
Embodiment 4: The method of any one of Embodiments 1-3, wherein the modification of the target locus of interest comprises a strand break.
Embodiment 5: The method of any one of Embodiments 1-4, wherein the effector protein and one or more nucleic acid components are non-naturally occurring.
Embodiment 6: The method of any one of Embodiments 1-5, wherein the effector protein is encoded by a subtype VI-D CRISPR-Cas loci.
Embodiment 7: The method of Embodiment 6, wherein the effector protein comprises Cas13d.
Embodiment 8: The method of Embodiment 7, wherein the Cas13d is derived from Ruminococcus flavefaciens.
Embodiment 9: The method of any one of Embodiments 1-8, wherein the effector protein is fused to one or more localization signal.
Embodiment 10: The method of Embodiment 9, wherein the one or more localization signal is nuclear localization signal.
Embodiment 11: The method of any one of the preceding Embodiments, wherein when in complex with the effector protein the nucleic acid component(s) is capable of effecting or effects sequence specific binding of the complex to a target sequence of the target locus of interest.
Embodiment 12: The method of any one of the preceding Embodiments, wherein the nucleic acid component(s) comprise one or more CRISPR RNA (crRNA) arrays and/or one or more trans-activating crRNA (tracrRNA).
Embodiment 13: The method of any one of the preceding Embodiments, wherein the nucleic acid component(s) comprise one or more CRISPR RNA (crRNA) arrays and do not comprise any trans-activating crRNA (tracrRNA).
Embodiment 14: The method of Embodiments 12 or 13, wherein the one or more CRISPR RNA (crRNA) arrays are pre-crRNA arrays.
Embodiment 15: The method of any one of the preceding Embodiments, wherein the effector protein and nucleic acid component(s) are provided via one or more polynucleotide molecules encoding the effector protein and/or the nucleic acid component(s), and wherein the one or more polynucleotide molecules are operably configured to express the effector protein and/or the nucleic acid component(s).
Embodiment 16: The method of Embodiment 15, wherein the one or more polynucleotide molecules comprise one or more regulatory elements operably configured to express the effector protein and/or the nucleic acid component(s).
Embodiment 17: The method of Embodiment 16, wherein the one or more regulatory elements are ubiquitous promoters or inducible promotors.
Embodiment 18: The method of Embodiment 17, wherein the one or more regulatory elements comprise one or more inducible UAS promoters.
Embodiment 19: The method of any one of Embodiments 15-18, wherein the one or more polynucleotide molecules are comprised within one or more vectors.
Embodiment 20: The method of any one of Embodiments 15-18, wherein the one or more polynucleotide molecules are comprised in a delivery system, or the method of claim 19 wherein the one or more vectors are comprised in a delivery system.
Embodiment 21: The method of any one of the preceding Embodiments, wherein the effector protein and one or more nucleic acid component(s) are delivered via one or more delivery vehicles comprising liposome(s), particle(s), exosome(s), microvesicle(s), a gene-gun or one or more viral vectors.
Embodiment 22: The method of any one of the preceding Embodiments, wherein the organism is a vertebrate.
Embodiment 23: The method of any one of the preceding Embodiments, wherein the organism is an invertebrate.
Embodiment 24: The method of Embodiment 23, wherein the organism is an insect.
Embodiment 25: An organism comprising a modified target locus of interest, wherein the target locus of interest has been modified according to a method of any one of the preceding Embodiments.
Embodiment 26: The organism of Embodiment 26, wherein the organism is a vertebrate.
Embodiment 27: The organism of Embodiment 26, wherein the organism is an invertebrate.
Embodiment 28: The organism of Embodiment 27, wherein the organism is an insect.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

TABLE 1
Transgenic Lines Used in this study

| | | | | | | Instegration | | | |
|---|---|---|---|---|---|---|---|---|---|
| Stock | | Transgensis | Target | Promoter/ | Genetic | Site, | Addgene | Bloomington | |
| Name | Name | Marker | Gene | 3′ UTR | Status | Chromsome | # | Fly Stock # | Source |
| OA- | Ubiq-CasRx | OpIE2- | N/A | dmelUbiquitin/ | Hetero- | attp40w | 132416 | 84118 | This study |
| 1050E.2 | | dsRed | | p10UTR | zygous | (2nd | | | |
| | | | | | balanced | chrom) | | | |
| | | | | | (CyO) | | | | |
| OA- | Ubiq-dCasRx | OpIE2- | N/A | dmelUbiquitin/ | Hetero- | attp40w | 132417 | 84119 | This study |
| 1050R | | dsRed | | p10UTR | zygous | (2nd | | | |
| | | | | | balanced | chrom) | | | |
| | | | | | (CyO) | | | | |
| OA- | UASt-CasRx | OpIE2- | N/A | UASt/p10UTR | Homozygous | 8621 (2nd | 132418 | 84121 | This study |
| 1050L | | dsRed | | | | chrom) | | | |
| OA- | UASt-dCasRx | OpIE2- | N/A | UASt/p10UTR | Homozygous | 8621 (2nd | 132419 | 84120 | This study |
| 1050S | | dsRed | | | | chrom) | | | |
| OA- | U6-3: | w+ | white | U6/U6 | Homozygous | 8622 (3rd | 132420 | 84124 | This study |
| 1050G | 4-gRNA-w | | | terminator | | chrom) | | | |
| OA- | U6-3: | w+ | notch | U6/U6 | Homozygous | 8622 (3rd | 132421 | 84122 | This study |
| 10501 | 4-gRNA-N | | | terminator | | chrom) | | | |
| OA- | U6-3: | w+; | GFP | U6/U6 | Homozygous | 8622 (3rd | 133304 | 84986 | This study |
| 1050J | 4-gRNA-GFP; | OpIE2- | | terminator | | chrom) | | | |
| | OpIE2-GFP | GFP | | | | | | | |
| OA- | U6-3: | w+ | Firefly | U6/U6 | Homozygous | 8622 (3rd | 132422 | 84125 | This study |
| 1050K | 4-gRNA-Fluc | | Luciferase | terminator | | chrom) | | | |
| OA- | U6-3: | w+ | cn | U6/U6 | Homozygous | 8622 (3rd | 132423 | 84126 | This study |
| 1050U | 4-gRNA-cn | | | terminator | | chrom) | | | |
| OA- | U6-3: | w+ | wg | U6/U6 | Homozygous | 8622 (3rd | 132424 | 84985 | This study |
| 1050V | 4-gRNA-wg | | | terminator | | chrom) | | | |
| OA- | U6-3: | w+ | y | U6/U6 | Homozygous | 8622 (3rd | 132425 | 84123 | This study |
| 1050Z4 | 4-gRNA-y | | | terminator | | chrom) | | | |
| OA- | Ubiq-Firefly- | w+ | N/A | Ubiq- | Homozygous | 9744 (3rd | 132426 | 84127 | This study |
| 1052B | T2A-eGFP- | | | Firefly-T2A- | | chrom) | | | |
| | Ubiquitin- | | | GFP/P10; Ubiq- | | | | | |
| | Renilla | | | Renilla/Sv40 | | | | | |
| 29967 | w[1118]; | w+ | N/A | GMR-Gal4 | Homozygous | Chr 3 | N/A | 29967 | Gunter Merdes, |
| | P{w[+mC] = | | | | | | | | University |
| | GAL4-ninaE.GMR}3, | | | | | | | | of Basel |
| | P{w[+m*] = | | | | | | | | |
| | lexAop-2 × | | | | | | | | |
| | hrGFP.nls}3b | | | | | | | | |
| 1560 | w[*]; | w+ | N/A | wg-Gal4 | Homozygous | Chr 2 | N/A | 1560 | Jean-Paul |
| | P{w[+mW.hs] = | | | | | | | | Vincent, MRC |
| | GAL4-arm.S}11 | | | | | | | | National |
| | | | | | | | | | Institute |
| | | | | | | | | | of Medical |
| | | | | | | | | | Research |
| 44373 | y[1] w[67c23]; | w+ | N/A | y-Gal4 | Homozygous | Chr 3 | N/A | 44373 | Craig Hart, |
| | P{w[+mC] = | | | | | | | | Louisiana State |
| | y-GAL4.G}3C | | | | | | | | University |

TABLE 2

| | | | | | CasRx | | Double |
|---|---|---|---|---|---|---|---|
| | | Cross (Cas | | | Transgene | Number | Transhet |
| Row | | Insertion | gRNA | CasRx | Insertion | of Double | with |
| number | Cross | Site) | Transgene | Transgene | Site | Transhet | Phenotype |
| 1 | Ubiq- | 1050G × | U6-3: | Ubiq- | attp40w | 10 | 10 |
| | CasRx; | 1050E.2(attp40w) | 4-gRNA-w | CasRx | | | |
| | gRNA- | | | | | | |
| | white | | | | | | |
| 2 | Ubiq- | 1050G × | U6-3: | Ubiq- | attp40w | 7 | 7 |
| | CasRx; | 1050E.2(attp40w) | 4-gRNA-w | CasRx | | | |
| | gRNA- | | | | | | |
| | white | | | | | | |
| 3 | Ubiq- | 1050G × | U6-3: | Ubiq- | attp40w | 22 | 22 |
| | CasRx; | 1050E.2(attp40w) | 4-gRNA-w | CasRx | | | |
| | gRNA- | | | | | | |
| | white | | | | | | |
| 4 | Ubiq- | 1050I × | U6-3: | Ubiq- | attp40w | 0 | 0 |
| | CasRx; | 1050E.2 (attp40w) | 4-gRNA-N | CasRx | | | |
| | gRNA- | | | | | | |
| | notch | | | | | | |
| 5 | Ubiq- | 1050I × | U6-3: | Ubiq- | attp40w | 0 | 0 |
| | CasRx: | 1050E.2 (attp40w) | 4-gRNA-N | CasRx | | | |
| | gRNA- | | | | | | |
| | notch | | | | | | |
| 6 | Ubiq- | 1050I × | U6-3: | Ubiq- | attp40w | 0 | 0 |
| | CasRx; | 1050E.2 (attp40w) | 4-gRNA-N | CasRx | | | |
| | gRNA- | | | | | | |
| | notch | | | | | | |
| 7 | Ubiq- | 1050U × | U6-3: | Ubiq- | attp40w | 11 | 8 |
| | CasRx: | 1050E.2(attp40w) | 4-gRNA-cn | CasRx | | | |
| | gRNA- | | | | | | |
| | cinnabar | | | | | | |
| 8 | Ubiq- | 1050U × | U6-3: | Ubiq- | attp40w | 55 | 47 |
| | CasRx; | 1050E.2(attp40w) | 4-gRNA-cn | CasRx | | | |
| | gRNA- | | | | | | |
| | cinnabar | | | | | | |
| 9 | Ubiq- | 1050U × | U6-3: | Ubiq- | attp40w | 53 | 45 |
| | CasRx; | 1050E.2(attp40w) | 4-gRNA-cn | CasRx | | | |
| | gRNA- | | | | | | |
| | cinnabar | | | | | | |
| 10 | Ubiq- | 1050V × | U6-3: | Ubiq- | attp40w | 2 | 1 |
| | CasRx; | 1050E.2(attp40w) | 4-gRNA-wg | CasRx | | | |
| | gRNA- | | | | | | |
| | wingless | | | | | | |
| 11 | Ubiq- | 1050V × | U6-3: | Ubiq- | attp40w | 10 | 7 |
| | CasRx; | 1050E.2(attp40w) | 4-gRNA-wg | CasRx | | | |
| | gRNA- | | | | | | |
| | wingless | | | | | | |
| 12 | Ubiq- | 1050V × | U6-3: | Ubiq- | attp40w | 5 | 3 |
| | CasRx; | 1050E.2(attp40w) | 4-gRNA-wg | CasRx | | | |
| | gRNA- | | | | | | |
| | wingless | | | | | | |
| 13 | Ubiq- | 1050Z4 × | U6-3: | Ubiq- | attp40w | 0 | 0 |
| | CasRx; | 1050E.2(attp40w) | 4-gRNA-y | CasRx | | | |
| | gRNA- | | | | | | |
| | yellow | | | | | | |
| 14 | Ubiq- | 1050Z4 × | U6-3: | Ubiq- | attp40w | 0 | 0 |
| | CasRx; | 1050E.2(attp40w) | 4-gRNA-y | CasRx | | | |
| | gRNA- | | | | | | |
| | yellow | | | | | | |
| 15 | Ubiq- | 1050Z4 × | U6-3: | Ubiq- | attp40w | 0 | 0 |
| | CasRx: | 1050E.2(attp40w) | 4-gRNA-y | CasRx | | | |
| | gRNA- | | | | | | |
| | yellow | | | | | | |
| 16 | Ubiq- | 1050J × | U6-3: | Ubiq- | attp40w | 0 | 0 |
| | CasRx; | 1050E.2(attp40w) | 4-gRNA- | CasRx | | | |
| | gRNA- | | GFP | | | | |
| | GFP | | | | | | |
| 17 | Ubiq- | 1050J × | U6-3: | Ubiq- | attp40w | 0 | 0 |
| | CasRx; | 1050E.2(attp40w) | 4-gRNA- | CasRx | | | |
| | gRNA- | | GFP | | | | |
| | GFP | | | | | | |
| 18 | Ubiq- | 1050J × | U6-3: | Ubiq- | attp40w | 0 | 0 |
| | CasRx; | 1050E.2(attp40w) | 4-gRNA- | CasRx | | | |
| | gRNA- | | GFP | | | | |
| | GFP | | | | | | |
| 19 | Ubiq- | 1050K × | U6-3: | Ubiq- | attp40w | 64 | 0 |
| | CasRx; | 1050E.2(attp40w) | 4-gRNA- | CasRx | | | |
| | gRNA- | | Fluc | | | | |
| | Fluc | | | | | | |
| 20 | Ubiq- | 1050K × | U6-3: | Ubiq- | attp40w | 54 | 0 |
| | CasRx; | 1050E.2(attp40w) | 4-gRNA- | CasRx | | | |
| | gRNA- | | Fluc | | | | |
| | Fluc | | | | | | |
| 21 | Ubiq- | 1050K × | U6-3: | Ubiq- | attp40w | 69 | 0 |
| | CasRx; | 1050E.2(attp40w) | 4-gRNA- | CasRx | | | |
| | gRNA- | | Fluc | | | | |
| | Fluc | | | | | | |
| 22 | Ubiq- | 1052B × | Ubiq-Fluc- | Ubiq- | attp40w | 86 | 0 |
| | CasRx; | 1050E.2(attp40w) | Ubiq-Rluc | CasRx | | | |
| | DLR | | | | | | |
| 23 | Ubiq- | 1052B × | Ubiq-Fluc- | Ubiq- | attp40w | 79 | 0 |
| | CasRx; | 1050E.2(attp40vv) | Ubiq-Rluc | CasRx | | | |
| | DLR | | | | | | |
| 24 | Ubiq- | 1052B × | Ubiq-Fluc- | Ubiq- | attp40w | 82 | 0 |
| | CasRx; | 1050E.2(attp40w) | Ubiq-Rluc | CasRx | | | |
| | DLR | | | | | | |
| 25 | Ubiq- | 1050G × | U6-3: | Ubiq- | attp40w | 41 | 0 |
| | dCasRx; | 1050R(attp40w) | 4-gRNA-w | dCasRx | | | |
| | gRNA- | | | | | | |
| | white | | | | | | |
| 26 | Ubiq- | 1050G × | U6-3: | Ubiq- | attp40w | 56 | 0 |
| | dCasRx; | 1050R(attp40w) | 4-gRNA-w | dCasRx | | | |
| | gRNA- | | | | | | |
| | white | | | | | | |
| 27 | Ubiq- | 1050G × | U6-3: | Ubiq- | attp40w | 51 | 0 |
| | dCasRx; | 1050R(attp40w) | 4-gRNA-w | dCasRx | | | |
| | gRNA- | | | | | | |
| | white | | | | | | |
| 28 | Ubiq- | 1050I × | U6-3: | Ubiq- | attp40w | 28 | 0 |
| | dCasRx; | 1050R(attp40w) | 4-gRNA-N | dCasRx | | | |
| | gRNA- | | | | | | |
| | notch | | | | | | |
| 29 | Ubiq- | 1050I × | U6-3: | Ubiq- | attp40w | 44 | 0 |
| | dCasRx; | 1050R(attp40w) | 4-gRNA-N | dCasRx | | | |
| | gRNA- | | | | | | |
| | notch | | | | | | |
| 30 | Ubiq- | 10501 × | U6-3: | Ubiq- | attp40w | 20 | 0 |
| | dCasRx; | 1050R(attp40w) | 4-gRNA-N | dCasRx | | | |
| | gRNA- | | | | | | |
| | notch | | | | | | |
| 31 | Ubiq- | 1050U × | U6-3: | Ubiq- | attp40w | 15 | 0 |
| | dCasRx: | 1050R(attp40w) | 4-gRNA-cn | dCasRx | | | |
| | gRNA- | | | | | | |
| | cinnabar | | | | | | |
| 32 | Ubiq- | 1050U × | U6-3: | Ubiq- | attp40w | 18 | 0 |
| | dCasRx; | 1050R(attp40w) | 4-gRNA-cn | dCasRx | | | |
| | gRNA- | | | | | | |
| | cinnabar | | | | | | |
| 33 | Ubiq- | 1050U × | U6-3: | Ubiq- | attp40w | 16 | 0 |
| | dCasRx; | 1050R(attp40w) | 4-gRNA-cn | dCasRx | | | |
| | gRNA- | | | | | | |
| | cinnabar | | | | | | |
| 34 | Ubiq- | 1050V × | U6-3: | Ubiq- | attp40w | 77 | 0 |
| | dCasRx; | 1050R(attp40w) | 4-gRNA-wg | dCasRx | | | |
| | gRNA- | | | | | | |
| | wingless | | | | | | |
| 35 | Ubiq- | 1050V × | U6-3: | Ubiq- | attp40w | 65 | 0 |
| | dCasRx; | 1050R(attp40w) | 4-gRNA-wg | dCasRx | | | |
| | gRNA- | | | | | | |
| | wingless | | | | | | |
| 36 | Ubiq- | 1050V × | U6-3: | Ubiq- | attp40w | 54 | 0 |
| | dCasRx; | 1050R(attp40w) | 4-gRNA-wg | dCasRx | | | |
| | gRNA- | | | | | | |
| | wingless | | | | | | |
| 37 | Ubiq- | 1050Z4 × | U6-3: | Ubiq- | attp40w | 51 | 0 |
| | dCasRx; | 1050R(attp40w) | 4-gRNA-y | dCasRx | | | |
| | gRNA- | | | | | | |
| | yellow | | | | | | |
| 38 | Ubiq- | 1050Z4 × | U6-3: | Ubiq- | attp40w | 30 | 0 |
| | dCasRx; | 1050R(attp40w) | 4-gRNA-y | dCasRx | | | |
| | gRNA- | | | | | | |
| | yellow | | | | | | |
| 39 | Ubiq- | 1050Z4 × | U6-3: | Ubiq- | attp40w | 62 | 0 |
| | dCasRx; | 1050R(attp40w) | 4-gRNA-y | dCasRx | | | |
| | gRNA- | | | | | | |
| | yellow | | | | | | |
| 40 | Ubiq- | 1050J × | U6-3: | Ubiq- | attp40w | 80 | 0 |
| | dCasRx; | 1050R(attp40w) | 4-gRNA- | dCasRx | | | |
| | gRNA- | | GFP | | | | |
| | GFP | | | | | | |
| 41 | Ubiq- | 1050J × | U6-3: | Ubiq- | attp40w | 87 | 0 |
| | dCasRx; | 1050R(attp40w) | 4-gRNA- | dCasRx | | | |
| | gRNA- | | GFP | | | | |
| | GFP | | | | | | |
| 42 | Ubiq- | 1050J × | U6-3: | Ubiq- | attp40w | 86 | 0 |
| | dCasRx; | 1050R(attp40w) | 4-gRNA- | dCasRx | | | |
| | gRNA- | | GFP | | | | |
| | GFP | | | | | | |
| 43 | Ubiq- | 1050K × | U6-3: | Ubiq- | attp40w | 65 | 0 |
| | dCasRx; | 1050R(attp40w) | 4-gRNA- | dCasRx | | | |
| | gRNA- | | Fluc | | | | |
| | Fluc | | | | | | |
| 44 | Ubiq- | 1050K × | U6-3: | Ubiq- | attp40w | 66 | 0 |
| | dCasRx; | 1050R(attp40w) | 4-gRNA- | dCasRx | | | |
| | gRNA- | | Fluc | | | | |
| | Fluc | | | | | | |
| 45 | Ubiq- | 1050K × | U6-3: | Ubiq- | attp40w | 58 | 0 |
| | dCasRx: | 1050R(attp40w) | 4-gRNA- | dCasRx | | | |
| | gRNA- | | Fluc | | | | |
| | Fluc | | | | | | |
| 46 | Ubiq- | 1050K × | Ubiq-Fluc- | Ubiq- | attp40w | 73 | 0 |
| | dCasRx; | 1050R(attp40w) | Ubiq-Rluc | dCasRx | | | |
| | DLR | | | | | | |
| 47 | Ubiq- | 1050K × | Ubiq-Fluc- | Ubiq- | attp40w | 82 | 0 |
| | dCasRx: | 1050R(attp40w) | Ubiq-Rluc | dCasRx | | | |
| | DLR | | | | | | |
| 48 | Ubiq- | 1050K × | Ubiq-Fluc- | Ubiq- | attp40w | 74 | 0 |
| | dCasRx; | 1050R(attp40w) | Ubiq-Rluc | dCasRx | | | |
| | DLR | | | | | | |
| 49 | Ubiq- | 1050G × | U6-3: | Ubiq- | attp40w | 20 | 20 |
| | CasRx; | 1050E.2(attp40w) | 4-gRNA-w | CasRx | | | |
| | gRNA- | | | | | | |
| | white | | | | | | |
| 50 | Ubiq- | 1050G × | U6-3: | Ubiq- | attp40w | 21 | 21 |
| | CasRx: | 1050E.2(attp40w) | 4-gRNA-w | CasRx | | | |
| | gRNA- | | | | | | |
| | white | | | | | | |
| 51 | Ubiq- | 1050G × | U6-3: | Ubiq- | attp40w | 33 | 33 |
| | CasRx; | 1050E.2(attp40w) | 4-gRNA-w | CasRx | | | |
| | gRNA- | | | | | | |
| | white | | | | | | |
| 52 | Ubiq- | 1050I × | U6-3: | Ubiq- | attp40w | 0 | 0 |
| | CasRx; | 1050E.2(attp40w) | 4-gRNA-N | CasRx | | | |
| | gRNA- | | | | | | |
| | notch | | | | | | |
| 53 | Ubiq- | 1050I × | U6-3: | Ubiq- | attp40w | 0 | 0 |
| | CasRx; | 1050E.2(attp40w) | 4-gRNA-N | CasRx | | | |
| | gRNA- | | | | | | |
| | notch | | | | | | |
| 54 | Ubiq- | 1050I × | U6-3: | Ubiq- | attp40w | 0 | 0 |
| | CasRx; | 1050E.2(attp40w) | 4-gRNA-N | CasRx | | | |
| | gRNA- | | | | | | |
| | notch | | | | | | |
| 55 | Ubiq- | 1050U × | U6-3: | Ubiq- | attp40w | 69 | 63 |
| | CasRx; | 1050E.2(attp40w) | 4-gRNA-cn | CasRx | | | |
| | gRNA- | | | | | | |
| | cinnabar | | | | | | |
| 56 | Ubiq- | 1050U × | U6-3: | Ubiq- | attp40w | 62 | 51 |
| | CasRx; | 1050E.2(attp40w) | 4-gRNA-cn | CasRx | | | |
| | gRNA- | | | | | | |
| | cinnabar | | | | | | |
| 57 | Ubiq- | 1050U × | U6-3: | Ubiq- | attp40w | 66 | 59 |
| | CasRx; | 1050E.2(attp40w) | 4-gRNA-cn | CasRx | | | |
| | gRNA- | | | | | | |
| | cinnabar | | | | | | |
| 58 | Ubiq- | 1050V × | U6-3: | Ubiq- | attp40w | 24 | 18 |
| | CasRx; | 1050E.2(attp40w) | 4-gRNA-wg | CasRx | | | |
| | gRNA- | | | | | | |
| | wingless | | | | | | |
| 59 | Ubiq- | 1050V × | U6-3: | Ubiq- | attp40w | 21 | 13 |
| | CasRx; | 1050E.2(attp40w) | 4-gRNA-wg | CasRx | | | |
| | gRNA- | | | | | | |
| | wingless | | | | | | |
| 60 | Ubiq- | 1050V × | U6-3: | Ubiq- | attp40w | 31 | 22 |
| | CasRx; | 1050E.2(attp40w) | 4-gRNA-wg | CasRx | | | |
| | gRNA- | | | | | | |
| | wingless | | | | | | |
| 61 | Ubiq- | 1050Z4 × | U6-3: | Ubiq- | attp40w | 0 | 0 |
| | CasRx; | 1050E.2(attp40w) | 4-gRNA-y | CasRx | | | |
| | gRNA- | | | | | | |
| | yellow | | | | | | |
| 62 | Ubiq- | 1050Z4 × 1050E.2(attp40w) | U6-3: | Ubiq- | attp40w | 0 | 0 |
| | CasRx; | | 4-gRNA-y | CasRx | | | |
| | gRNA- | | | | | | |
| | yellow | | | | | | |
| 63 | Ubiq- | 1050Z4 × 1050E.2(attp40w) | U6-3: | Ubiq- | attp40w | 0 | 0 |
| | CasRx; | | 4-gRNA-y | CasRx | | | |
| | gRNA- | | | | | | |
| | yellow | | | | | | |
| 64 | Ubiq- | 10501 × | U6-3: | Ubiq- | attp40w | 0 | 0 |
| | CasRx; | 1050E.2(attp40w) | 4-gRNA- | CasRx | | | |
| | gRNA- | | GFP | | | | |
| | GFP | | | | | | |
| 65 | Ubiq- | 10501 × | U6-3: | Ubiq- | attp40w | 0 | 0 |
| | CasRx; | 1050E.2(attp40w) | 4-gRNA- | CasRx | | | |
| | gRNA- | | GFP | | | | |
| | GFP | | | | | | |
| 66 | Ubiq- | 10501 × | U6-3: | Ubiq- | attp40w | 0 | 0 |
| | CasRx; | 1050E.2(attp40w) | 4-gRNA- | CasRx | | | |
| | gRNA- | | GFP | | | | |
| | GFP | | | | | | |
| 67 | Ubiq- | 1050G × | U6-3: | Ubiq- | attp40w | 54 | 0 |
| | dCasRx; | 1050R(attp40w) | 4-gRNA-w | dCasRx | | | |
| | gRNA- | | | | | | |
| | white | | | | | | |
| 68 | Ubiq- | 1050G × | U6-3: | Ubiq- | attp40w | 45 | 0 |
| | dCasRx; | 1050R(attp40w) | 4-gRNA-w | dCasRx | | | |
| | gRNA- | | | | | | |
| | white | | | | | | |
| 69 | Ubiq- | 1050G × | U6-3: | Ubiq- | attp40w | 49 | 0 |
| | dCasRx; | 1050R(attp40w) | 4-gRNA-w | dCasRx | | | |
| | gRNA- | | | | | | |
| | white | | | | | | |
| 70 | Ubiq- | 10501 × | U6-3: | Ubiq- | attp40w | 30 | 0 |
| | dCasRx; | 1050R(attp40w) | 4-gRNA-N | dCasRx | | | |
| | gRNA- | | | | | | |
| | notch | | | | | | |
| 71 | Ubiq- | 10501 × | U6-3: | Ubiq- | attp40w | 26 | 0 |
| | dCasRx; | 1050R(attp40w) | 4-gRNA-N | dCasRx | | | |
| | gRNA- | | | | | | |
| | notch | | | | | | |
| 72 | Ubiq- | 10501 × | U6-3: | Ubiq- | attp40w | 28 | 0 |
| | dCasRx; | 1050R(attp40w) | 4-gRNA-N | dCasRx | | | |
| | gRNA- | | | | | | |
| | notch | | | | | | |
| 73 | Ubiq- | 1050U × | U6-3: | Ubiq- | attp40w | 55 | 0 |
| | dCasRx; | 1050R(attp40w) | 4-gRNA-cn | dCasRx | | | |
| | gRNA- | | | | | | |
| | cinnabar | | | | | | |
| 74 | Ubiq- | 1050U × | U6-3: | Ubiq- | attp40w | 58 | 0 |
| | dCasRx; | 1050R(attp40w) | 4-gRNA-cn | dCasRx | | | |
| | gRNA- | | | | | | |
| | cinnabar | | | | | | |
| 75 | Ubiq- | 1050U × | U6-3: | Ubiq- | attp40w | 57 | 0 |
| | dCasRx; | 1050R(attp40w) | 4-gRNA-cn | dCasRx | | | |
| | gRNA- | | | | | | |
| | cinnabar | | | | | | |
| 76 | Ubiq- | 1050V × | U6-3: | Ubiq- | attp40w | 39 | 0 |
| | dCasRx; | 1050R(attp40w) | 4-gRNA-wg | dCasRx | | | |
| | gRNA- | | | | | | |
| | wingless | | | | | | |
| 77 | Ubiq- | 1050V × | U6-3: | Ubiq- | attp40w | 22 | 0 |
| | dCasRx; | 1050R(attp40w) | 4-gRNA-wg | dCasRx | | | |
| | gRNA- | | | | | | |
| | wingless | | | | | | |
| 78 | Ubiq- | 1050V × | U6-3: | Ubiq- | attp40w | 28 | 0 |
| | dCasRx; | 1050R(attp40w) | 4-gRNA-wg | dCasRx | | | |
| | gRNA- | | | | | | |
| | wingless | | | | | | |
| 79 | Ubiq- | 1050Z4 × | U6-3: | Ubiq- | attp40w | 81 | 0 |
| | dCasRx; | 1050R(attp40w) | 4-gRNA-y | dCasRx | | | |
| | gRNA- | | | | | | |
| | yellow | | | | | | |
| 80 | Ubiq- | 1050Z4 × | U6-3: | Ubiq- | attp40w | 69 | 0 |
| | dCasRx; | 1050R(attp40w) | 4-gRNA-y | dCasRx | | | |
| | gRNA- | | | | | | |
| | yellow | | | | | | |
| 81 | Ubiq- | 1050Z4 × | U6-3: | Ubiq- | attp40w | 60 | 0 |
| | dCasRx; | 1050R(attp40w) | 4-gRNA-y | dCasRx | | | |
| | gRNA- | | | | | | |
| | yellow | | | | | | |
| 82 | Ubiq- | 1050J × | U6-3: | Ubiq- | attp40w | 48 | 0 |
| | dCasRx; | 1050R(attp40w) | 4-gRNA- | dCasRx | | | |
| | gRNA- | | GFP | | | | |
| | GFP | | | | | | |
| 83 | Ubiq- | 1050J × | U6-3: | Ubiq- | attp40w | 83 | 0 |
| | dCasRx; | 1050R(attp40w) | 4-gRNA- | dCasRx | | | |
| | gRNA- | | GFP | | | | |
| | GFP | | | | | | |
| 84 | Ubiq- | 1050J × | U6-3: | Ubiq- | attp40w | 85 | 0 |
| | dCasRx; | 1050R(attp40w) | 4-gRNA- | dCasRx | | | |
| | gRNA- | | GFP | | | | |
| | GFP | | | | | | |

| | Number of | Double | | | |
|---|---|---|---|---|---|
| Row | gRNA-only | Transhet | gRNA-only | Total | Cross |
| number | Progeny | Ratio | Ratio | Progeny | Type |
| 1 | 104 | 8.771929825 | 91.22807018 | 114 | Paternal |
| 2 | 158 | 4.242424242 | 95.75757576 | 165 | Paternal |
| 3 | 168 | 11.57894737 | 88.42105263 | 190 | Paternal |
| 4 | 213 | 0 | 100 | 213 | Paternal |
| 5 | 155 | 0 | 100 | 155 | Paternal |
| 6 | 197 | 0 | 100 | 197 | Paternal |
| 7 | 105 | 9.482758621 | 90.51724138 | 116 | Paternal |
| 8 | 176 | 23.80952381 | 76.19047619 | 231 | Paternal |
| 9 | 212 | 20 | 80 | 265 | Paternal |
| 10 | 94 | 2.083333333 | 97.91666667 | 96 | Paternal |
| 11 | 122 | 7.575757576 | 92.42424242 | 132 | Paternal |
| 12 | 134 | 3.597122302 | 96.4028777 | 139 | Paternal |
| 13 | 199 | 0 | 100 | 199 | Paternal |
| 14 | 256 | 0 | 100 | 256 | Paternal |
| 15 | 167 | 0 | 100 | 167 | Paternal |
| 16 | 185 | 0 | 100 | 185 | Paternal |
| 17 | 173 | 0 | 100 | 173 | Paternal |
| 18 | 169 | 0 | 100 | 169 | Paternal |
| 19 | 103 | 38.32335329 | 61.67664671 | 167 | Paternal |
| 20 | 100 | 35.06493506 | 64.93506494 | 154 | Paternal |
| 21 | 104 | 39.88439306 | 60.11560694 | 173 | Paternal |
| 22 | 94 | 47.77777778 | 52.22222222 | 180 | Paternal |
| 23 | 84 | 48.46625767 | 51.53374233 | 163 | Paternal |
| 24 | 98 | 45.55555556 | 54.44444444 | 180 | Paternal |
| 25 | 115 | 26.28205128 | 73.71794872 | 156 | Paternal |
| 26 | 127 | 30.6010929 | 69.3989071 | 183 | Paternal |
| 27 | 109 | 31.875 | 68.125 | 160 | Paternal |
| 28 | 166 | 14.43298969 | 85.56701031 | 194 | Paternal |
| 29 | 195 | 18.41004184 | 81.58995816 | 239 | Paternal |
| 30 | 124 | 13.88888889 | 86.11111111 | 144 | Paternal |
| 31 | 155 | 8.823529412 | 91.17647059 | 170 | Paternal |
| 32 | 130 | 12.16216216 | 87.83783784 | 148 | Paternal |
| 33 | 124 | 11.42857143 | 88.57142857 | 140 | Paternal |
| 34 | 122 | 38.69346734 | 61.30653266 | 199 | Paternal |
| 35 | 111 | 36.93181818 | 63.06818182 | 176 | Paternal |
| 36 | 107 | 33.54037267 | 66.45962733 | 161 | Paternal |
| 37 | 156 | 24.63768116 | 75.36231884 | 207 | Paternal |
| 38 | 106 | 22.05882353 | 77.94117647 | 136 | Paternal |
| 39 | 149 | 29.38388626 | 70.61611374 | 211 | Paternal |
| 40 | 102 | 43.95604396 | 56.04395604 | 182 | Paternal |
| 41 | 125 | 41.03773585 | 58.96226415 | 212 | Paternal |
| 42 | 130 | 39.81481481 | 60.18518519 | 216 | Paternal |
| 43 | 107 | 37.79069767 | 62.20930233 | 172 | Paternal |
| 44 | 93 | 41.50943396 | 58.49056604 | 159 | Paternal |
| 45 | 91 | 38.9261745 | 61.0738255 | 149 | Paternal |
| 46 | 80 | 47.7124183 | 52.2875817 | 153 | Paternal |
| 47 | 78 | 51.25 | 48.75 | 160 | Paternal |
| 48 | 85 | 46.5408805 | 53.4591195 | 159 | Paternal |
| 49 | 127 | 13.60544218 | 86.39455782 | 147 | Maternal |
| 50 | 117 | 15.2173913 | 84.7826087 | 138 | Maternal |
| 51 | 102 | 24.44444444 | 75.55555556 | 135 | Maternal |
| 52 | 134 | 0 | 100 | 134 | Maternal |
| 53 | 122 | 0 | 100 | 122 | Maternal |
| 54 | 148 | 0 | 100 | 148 | Maternal |
| 55 | 107 | 39.20454545 | 60.79545455 | 176 | Maternal |
| 56 | 103 | 37.57575758 | 62.42424242 | 165 | Maternal |
| 57 | 97 | 40.49079755 | 59.50920245 | 163 | Maternal |
| 58 | 192 | 11.11111111 | 88.88888889 | 216 | Maternal |
| 59 | 125 | 14.38356164 | 85.61643836 | 146 | Maternal |
| 60 | 134 | 18.78787879 | 81.21212121 | 165 | Maternal |
| 61 | 117 | 0 | 100 | 117 | Maternal |
| 62 | 102 | 0 | 100 | 102 | Maternal |
| 63 | 129 | 0 | 100 | 129 | Maternal |
| 64 | 106 | 0 | 100 | 106 | Maternal |
| 65 | 136 | 0 | 100 | 136 | Maternal |
| 66 | 115 | 0 | 100 | 115 | Maternal |
| 67 | 118 | 31.39534884 | 68.60465116 | 172 | Maternal |
| 68 | 129 | 25.86206897 | 74.13793103 | 174 | Maternal |
| 69 | 125 | 28.16091954 | 71.83908046 | 174 | Maternal |
| 70 | 100 | 23.07692308 | 76.92307692 | 130 | Maternal |
| 71 | 101 | 20.47244094 | 79.52755906 | 127 | Maternal |
| 72 | 105 | 21.05263158 | 78.94736842 | 133 | Maternal |
| 73 | 108 | 33.74233129 | 66.25766871 | 163 | Maternal |
| 74 | 106 | 35.36585366 | 64.63414634 | 164 | Maternal |
| 75 | 104 | 35.40372671 | 64.59627329 | 161 | Maternal |
| 76 | 201 | 16.25 | 83.75 | 240 | Maternal |
| 77 | 112 | 16.41791045 | 83.58208955 | 134 | Maternal |
| 78 | 114 | 19.71830986 | 80.28169014 | 142 | Maternal |
| 79 | 115 | 41.32653061 | 58.67346939 | 196 | Maternal |
| 80 | 111 | 38.33333333 | 61.66666667 | 180 | Maternal |
| 81 | 110 | 35.29411765 | 64.70588235 | 170 | Maternal |
| 82 | 110 | 30.37974684 | 69.62025316 | 158 | Maternal |
| 83 | 156 | 34.72803347 | 65.27196653 | 239 | Maternal |
| 84 | 162 | 34.41295547 | 65.58704453 | 247 | Maternal |

TABLE 3

| | Column Number | | | | | |
|---|---|---|---|---|---|---|
| | | 2 | | | | |
| | | Cross | 3 | 4 | 5 | 6 |
| Row | 1 | (Internal | Gal4 | gRNA | CasRx | Replicate |
| Number | Cross | Reference) | Transgene | Transgene | Transgene | No. |
| 1 | armadillo-Gal4 −> UASt-CasRx; | 1560 × | armadillo | U6-3: | UASt-CasRx | 1 |
| | gRNA-wingless | 1050V:1050L(8621) | | 4-gRNA-wg | | |
| 2 | armadillo-Gal4 −> UASt-CasRx; | 1560 × | armadillo | U6-3: | UASt-CasRx | 2 |
| | gRNA-wingless | 1050V:1050L(8621) | | 4-gRNA-wg | | |
| 3 | armadillo-Gal4 −> UASt-CasRx; | 1560 × | armadillo | U6-3: | UASt-CasRx | 3 |
| | gRNA-wingless | 1050V:1050L(8621) | | 4-gRNA-wg | | |
| 4 | armadillo-Gal4 −> UASt-dCasRx; | 1560 × | armadillo | U6-3: | UASt-dCasRx | 1 |
| | gRNA-wingless | 1050V:1050S(8621) | | 4-gRNA-wg | | |
| 5 | armadillo-Gal4 −> UASt-dCasRx; | 1560 × | armadillo | U6-3: | UASt-dCasRx | 2 |
| | gRNA-wingless | 1050V:1050S(8621) | | 4-gRNA-wg | | |
| 6 | armadillo-Gal4 −> UASt-dCasRx; | 1560 × | armadillo | U6-3: | UASt-dCasRx | 3 |
| | gRNA-wingless | 1050V:1050S(8621) | | 4-gRNA-wg | | |
| 7 | GMR2-Gal4 −> UASt-CasRx; | 29967 × | GMR2 | U6-3: | UASt-CasRx | 1 |
| | gRNA-white | 1050G:1050L(8621) | | 4-gRNA-w | | |
| 8 | GMR2-Gal4 −> UASt-CasRx; | 29967 × | GMR2 | U6-3: | UASt-CasRx | 2 |
| | gRNA-white | 1050G:1050L(8621) | | 4-gRNA-w | | |
| 9 | GMR2-Gal4 −> UASt-CasRx; | 29967 × | GMR2 | U6-3: | UASt-CasRx | 3 |
| | gRNA-white | 1050G:1050L(8621) | | 4-gRNA-w | | |
| 10 | GMR2-Gal4 −> UASt-dCasRx; | 29967 × | GMR2 | U6-3: | UASt-dCasRx | 1 |
| | gRNA-white | 1050G:1050S(8621) | | 4-gRNA-w | | |
| 11 | GMR2-Gal4 −> UASt-dCasRx; | 29967 × | GMR2 | U6-3: | UASt-dCasRx | 2 |
| | gRNA-white | 1050G:1050S(8621) | | 4-gRNA-w | | |
| 12 | GMR2-Gal4 −> UASt-dCasRx; | 29967 × | GMR2 | U6-3: | UASt-dCasRx | 3 |
| | gRNA-white | 1050G:1050S(8621) | | 4-gRNA-w | | |
| 13 | GMR2-Gal4 −> UASt-CasRx; | 29967 × | GMR2 | U6-3: | UASt-CasRx | 1 |
| | gRNA-notch | 1050I:1050L(8621) | | 4-gRNA-N | | |
| 14 | GMR2-Gal4 −> UASt-CasRx; | 29967 × | GMR2 | U6-3: | UASt-CasRx | 2 |
| | gRNA-notch | 1050I:1050L(8621) | | 4-gRNA-N | | |
| 15 | GMR2-Gal4 −> UASt-CasRx; | 29967 × | GMR2 | U6-3: | UASt-CasRx | 3 |
| | gRNA-notch | 1050I:1050L(8621) | | 4-gRNA-N | | |
| 16 | GMR2-Gal4 −> UASt-dCasRx; | 29967 × | GMR2 | U6-3: | UASt-dCasRx | 1 |
| | gRNA-notch | 1050I:1050S(8621) | | 4-gRNA-N | | |
| 17 | GMR2-Gal4 −> UASt-dCasRx; | 29967 × | GMR2 | U6-3: | UASt-dCasRx | 2 |
| | gRNA-notch | 1050I:1050S(8621) | | 4-gRNA-N | | |
| 18 | GMR2-Gal4 −> UASt-dCasRx; | 29967 × | GMR2 | U6-3: | UASt-dCasRx | 3 |
| | gRNA-notch | 1050I:1050S(8621) | | 4-gRNA-N | | |
| 19 | GMR2-Gal4 −> UASt-CasRx; | 29967 × | GMR2 | U6-3: | UASt-CasRx | 1 |
| | gRNA-cinnabar | 1050U:1050L(8621) | | 4-gRNA-cn | | |
| 20 | GMR2-Gal4 −> UASt-CasRx; | 29967 × | GMR2 | U6-3: | UASt-CasRx | 2 |
| | gRNA-cinnabar | 1050U:1050L(8621) | | 4-gRNA-cn | | |
| 21 | GMR2-Gal4 −> UASt-CasRx; | 29967 × | GMR2 | U6-3: | UASt-CasRx | 3 |
| | gRNA-cinnabar | 1050U:1050L(8621) | | 4-gRNA-cn | | |
| 22 | GMR2-Gal4 −> UASt-dCasRx; | 29967 × | GMR2 | U6-3: | UASt-dCasRx | 1 |
| | gRNA-cinnabar | 1050U:1050S(8621) | | 4-gRNA-cn | | |
| 23 | GMR2-Gal4 −> UASt-dCasRx; | 29967 × | GMR2 | U6-3: | UASt-dCasRx | 2 |
| | gRNA-cinnabar | 1050U:1050S(8621) | | 4-gRNA-cn | | |
| 24 | GMR2-Gal4 −> UASt-dCasRx; | 29967 × | GMR2 | U6-3: | UASt-dCasRx | 3 |
| | gRNA-cinnabar | 1050U:1050S(8621) | | 4-gRNA-cn | | |
| 25 | y2-Gal4 −> UASt-CasRx; | 44373 × | y2 | U6-3: | UASt-CasRx | 1 |
| | gRNA-yellow | 1050Z4:1050L(8621) | | 4-gRNA-y | | |
| 26 | y2-Gal4 −> UASt-CasRx; | 44373 × | y2 | U6-3: | UASt-CasRx | 2 |
| | gRNA-yellow | 1050Z4:1050L(8621) | | 4-gRNA-y | | |
| 27 | y2-Gal4 −> UASt-CasRx; | 44373 × | y2 | U6-3: | UASt-CasRx | 3 |
| | gRNA-yellow | 1050Z4:1050L(8621) | | 4-gRNA-y | | |
| 28 | y2-Gal4 −> UASt-dCasRx; | 44373 × | y2 | U6-3: | UASt-dCasRx | 1 |
| | gRNA-yellow | 1050Z4:1050S(8621) | | 4-gRNA-y | | |
| 29 | y2-Gal4 −> UASt-dCasRx; | 44373 × | y2 | U6-3: | UASt-dCasRx | 2 |
| | gRNA-yellow | 1050Z4:1050S(8621) | | 4-gRNA-y | | |
| 30 | y2-Gal4 −> UASt-dCasRx; | 44373 × | y2 | U6-3: | UASt-dCasRx | 3 |
| | gRNA-yellow | 1050Z4:1050S(8621) | | 4-gRNA-y | | |
| 31 | y2-Gal4 −> UASt-dCasRx; | 44373 × | y2 | U6-3: | UASt-dCasRx | 4 |
| | gRNA-yellow | 1050Z4:1050S(8621) | | 4-gRNA-y | | |

Column Number

| | | | 9 | |
|---|---|---|---|---|
| | | | Number of | |
| | | 8 | Triple Transhets | 10 |
| | 7 | Number of | Manifesting | Triple |
| Row | Number of | Triple | Distinct | Transhets |
| Number | All Progeny | Transhets | Phenotypes | Ratios |
| 1 | 189 | 0 | 0 | 0 |
| 2 | 162 | 0 | 0 | 0 |
| 3 | 112 | 0 | 0 | 0 |
| 4 | 126 | 39 | 0 | 0.309524 |
| 5 | 125 | 41 | 0 | 0.328 |
| 6 | 182 | 46 | 0 | 0.252747 |
| 7 | 234 | 4 | 4 | 0.017094 |
| 8 | 181 | 0 | 0 | 0 |
| 9 | 98 | 0 | 0 | 0 |
| 10 | 93 | 22 | 0 | 0.236559 |
| 11 | 104 | 28 | 0 | 0.269231 |
| 12 | 106 | 34 | 0 | 0.320755 |
| 13 | 150 | 0 | 0 | 0 |
| 14 | 83 | 0 | 0 | 0 |
| 15 | 88 | 0 | 0 | 0 |
| 16 | 31 | 8 | 0 | 0.258065 |
| 17 | 120 | 39 | 0 | 0.325 |
| 18 | 96 | 25 | 0 | 0.260417 |
| 19 | 214 | 54 | 54 | 0.252336 |
| 20 | 82 | 26 | 26 | 0.317073 |
| 21 | 115 | 31 | 31 | 0.269565 |
| 22 | 145 | 40 | 0 | 0.275862 |
| 23 | 173 | 51 | 0 | 0.294798 |
| 24 | 153 | 41 | 0 | 0.267974 |
| 25 | 210 | 3 | 3 | 0.014286 |
| 26 | 169 | 10 | 8 | 0.059172 |
| 27 | 211 | 2 | 2 | 0.009479 |
| 28 | 227 | 75 | 0 | 0.330396 |
| 29 | 32 | 6 | 0 | 0.1875 |
| 30 | 105 | 25 | 0 | 0.238095 |
| 31 | 40 | 17 | 0 | 0.425 |

| | Column Number | | | | | | | | | |
|---|---|---|---|---|---|---|---|---|---|---|
| | 11 | | | | | | | | | |
| | Distinct | | | | | | 17 | | | |
| | Phenotype | 12 | 13 | 14 | 15 | 16 | Ratio of | | | 20 |
| | Penentrance | Number of | Number of | Number of | Ratio of | Ratio of | Non-CasRx, | 18 | | Mean |
| Row | Among Triple | CasRx, | Non-CasRx, | Non-CasRx, | CasRx, | Non-CasRx, | Non- | gRNA | 19 | Transhet |
| Number | Transhets | Stubble | Stubble | Non-Stubble | Stubble | Stubble | Stubble | Target | Cross Type | Penetrance |
| 1 | NA | 42 | 74 | 73 | 0.222222 | 0.391534 | 0.386243 | wingless | armadillo- | 1 |
| | | | | | | | | | wingless | |
| 2 | NA | 33 | 44 | 85 | 0.203704 | 0.271605 | 0.524691 | wingless | armadillo- | 1 |
| | | | | | | | | | wingless | |
| 3 | NA | 32 | 24 | 56 | 0.285714 | 0.214286 | 0.5 | wingless | armadillo- | 1 |
| | | | | | | | | | wingless | |
| 4 | 0 | 23 | 29 | 35 | 0.18254 | 0.230159 | 0.277778 | wingless | armadillo- | 0 |
| | | | | | | | | | wingless | |
| 5 | 0 | 18 | 26 | 40 | 0.144 | 0.208 | 0.32 | wingless | armadillo- | 0 |
| | | | | | | | | | wingless | |
| 6 | 0 | 26 | 39 | 71 | 0.142857 | 0.214286 | 0.39011 | wingless | armadillo- | 0 |
| | | | | | | | | | wingless | |
| 7 | 1 | 70 | 68 | 92 | 0.299145 | 0.290598 | 0.393162 | white | GMR2-white | 1 |
| 8 | NA | 54 | 66 | 61 | 0.298343 | 0.364641 | 0.337017 | white | GMR2-white | 1 |
| 9 | NA | 26 | 36 | 36 | 0.265306 | 0.367347 | 0.367347 | white | GMR2-white | 1 |
| 10 | 0 | 26 | 25 | 20 | 0.27957 | 0.268817 | 0.215054 | white | GMR2-white | 0 |
| 11 | 0 | 18 | 19 | 39 | 0.173077 | 0.182692 | 0.375 | white | GMR2-white | 0 |
| 12 | 0 | 12 | 24 | 36 | 0.113208 | 0.226415 | 0.339623 | white | GMR2-white | 0 |
| 13 | NA | 44 | 44 | 62 | 0.293333 | 0.293333 | 0.413333 | notch | GMR2-notch | 1 |
| 14 | NA | 24 | 24 | 35 | 0.289157 | 0.289157 | 0.421687 | notch | GMR2-notch | 1 |
| 15 | NA | 27 | 20 | 41 | 0.306818 | 0.227273 | 0.465909 | notch | GMR2-notch | 1 |
| 16 | 0 | 7 | 8 | 8 | 0.225806 | 0.258065 | 0.258065 | notch | GMR2-notch | 0 |
| 17 | 0 | 20 | 24 | 37 | 0.166667 | 0.2 | 0.308333 | notch | GMR2-notch | 0 |
| 18 | 0 | 18 | 22 | 31 | 0.1875 | 0.229167 | 0.322917 | notch | GMR2-notch | 0 |
| 19 | 1 | 51 | 50 | 59 | 0.238318 | 0.233645 | 0.275701 | cinnabar | GMR2- | 1 |
| | | | | | | | | | cinnabar | |
| 20 | 1 | 15 | 22 | 19 | 0.182927 | 0.268293 | 0.231707 | cinnabar | GMR2- | 1 |
| | | | | | | | | | cinnabar | |
| 21 | 1 | 23 | 20 | 41 | 0.2 | 0.173913 | 0.356522 | cinnabar | GMR2- | 1 |
| | | | | | | | | | cinnabar | |
| 22 | 0 | 34 | 31 | 40 | 0.234483 | 0.213793 | 0.275862 | cinnabar | GMR2- | 0 |
| | | | | | | | | | cinnabar | |
| 23 | 0 | 31 | 43 | 48 | 0.179191 | 0.248555 | 0.277457 | cinnabar | GMR2- | 0 |
| | | | | | | | | | cinnabar | |
| 24 | 0 | 39 | 36 | 37 | 0.254902 | 0.235294 | 0.24183 | cinnabar | GMR2- | 0 |
| | | | | | | | | | cinnabar | |
| 25 | 1 | 44 | 61 | 102 | 0.209524 | 0.290476 | 0.485714 | yellow | y2-yellow | 0.933333 |
| 26 | 0.8 | 37 | 48 | 74 | 0.218935 | 0.284024 | 0.43787 | yellow | y2-yellow | 0.933333 |
| 27 | 1 | 54 | 63 | 92 | 0.255924 | 0.298578 | 0.436019 | yellow | y2-yellow | 0.933333 |
| 28 | 0 | 34 | 61 | 57 | 0.14978 | 0.268722 | 0.251101 | yellow | y2-yellow | 0 |
| 29 | 0 | 9 | 10 | 7 | 0.28125 | 0.3125 | 0.21875 | yellow | y2-yellow | 0 |
| 30 | 0 | 20 | 17 | 43 | 0.190476 | 0.161905 | 0.409524 | yellow | y2-yellow | 0 |
| 31 | 0 | 4 | 4 | 15 | 0.1 | 0.1 | 0.375 | yellow | y2-yellow | 0 |

TABLE 4

| | Column Number | | | | | |
|---|---|---|---|---|---|---|
| Row | 1 | 2 | 3 | 4 | 5 | 6 |
| Number | Sample Name | Luciferase.Transgene | gRNA | CasRx | Replicate | Firefly |
| 1 | Ubiq-Firefly_Ubiq-Renilla-dCasRx-1 | Ubiq-Firefly_Ubiq-Renilla | N/A | dCasRx | 1.00 | 185000000 |
| 2 | Ubiq-Firefly_Ubiq-Renilla-dCasRx-2 | Ubiq-Firefly_Ubiq-Renilla | N/A | dCasRx | 2.00 | 125000000 |
| 3 | Ubiq-Firefly_Ubiq-Renilla-dCasRx-3 | Ubiq-Firefly_Ubiq-Renilla | N/A | dCasRx | 3.00 | 194000000 |
| 4 | Ubiq-Firefly_Ubiq-Renilla-dCasRx-4 | Ubiq-Firefly_Ubiq-Renilla | N/A | dCasRx | 4.00 | 294000000 |
| 5 | Ubiq-Firefly_Ubiq-Renilla-dCasRx-5 | Ubiq-Firefly_Ubiq-Renilla | N/A | dCasRx | 5.00 | 335000000 |
| 6 | Ubiq-Firefly_Ubiq-Renilla-dCasRx-6 | Ubiq-Firefly_Ubiq-Renilla | N/A | dCasRx | 6.00 | 372000000 |
| 7 | Ubiq-Firefly_Ubiq-Renilla-dCasRx-7 | Ubiq-Firefly_Ubiq-Renilla | N/A | dCasRx | 7.00 | 187000000 |
| 8 | Ubiq-Firefly_Ubiq-Renilla-dCasRx-8 | Ubiq-Firefly_Ubiq-Renilla | N/A | dCasRx | 8.00 | 184000000 |
| 9 | Ubiq-Firefly_Ubiq-Renilla-dCasRx-9 | Ubiq-Firefly_Ubiq-Renilla | N/A | dCasRx | 9.00 | 221000000 |
| 10 | Ubiq-Firefly_Ubiq-Renilla-dCasRx-U6-3: | Ubiq-Firefly_Ubiq-Renilla | U6-3: | dCasRx | 1.00 | 293000000 |
| | 4-gRNA-Fluc-1 | | 4-gRNA-Fluc | | | |
| 11 | Ubiq-Firefly_Ubiq-Renilla-dCasRx-U6-3: | Ubiq-Firefly_Ubiq-Renilla | U6-3: | dCasRx | 2.00 | 261000000 |
| | 4-gRNA-Fluc-2 | | 4-gRNA-Fluc | | | |
| 12 | Ubiq-Firefly_Ubiq-Renilla-dCasRx-U6-3: | Ubiq-Firefly_Ubiq-Renilla | U6-3: | dCasRx | 3.00 | 356000000 |
| | 4-gRNA-Fluc-3 | | 4-gRNA-Fluc | | | |
| 13 | Ubiq-Firefly_Ubiq-Renilla-dCasRx-U6-3: | Ubiq-Firefly_Ubiq-Renilla | U6-3: | dCasRx | 4.00 | 383000000 |
| | 4-gRNA-Fluc- | | 4-gRNA-Fluc | | | |
| 14 | Ubiq-Firefly_Ubiq-Renilla-dCasRx-U6-3: | Ubiq-Firefly_Ubiq-Renilla | U6-3: | dCasRx | 5.00 | 413000000 |
| | 4-gRNA-Fluc-5 | | 4-gRNA-Fluc | | | |
| 15 | Ubiq-Firefly_Ubiq-Renilla-dCasRx-U6-3: | Ubiq-Firefly_Ubiq-Renilla | U6-3: | dCasRx | 6.00 | 357000000 |
| | 4-gRNA-Fluc-6 | | 4-gRNA-Fluc | | | |
| 16 | Ubiq-Firefly_Ubiq-Renilla-dCasRx-U6-3: | Ubiq-Firefly_Ubiq-Renilla | U6-3: | dCasRx | 7.00 | 302000000 |
| | 4-gRNA-Fluc-7 | | 4-gRNA-Fluc | | | |
| 17 | Ubiq-Firefly_Ubiq-Renilla-dCasRx-U6-3: | Ubiq-Firefly_Ubiq-Renilla | U6-3: | dCasRx | 8.00 | 308000000 |
| | 4-gRNA-Fluc-8 | | 4-gRNA-Fluc | | | |
| 18 | Ubiq-Firefly_Ubiq-Renilla-dCasRx-U6-3: | Ubiq-Firefly_Ubiq-Renilla | U6-3: | dCasRx | 9.00 | 277000000 |
| | 4-gRNA-Fluc-9 | | 4-gRNA-Fluc | | | |
| 19 | Ubiq-Firefly_Ubiq-Renilla-dCasRx-U6-3: | Ubiq-Firefly_Ubiq-Renilla | U6-3: | dCasRx | 10.00 | 372000000 |
| | 4-gRNA-Fluc-10 | | 4-gRNA-Fluc | | | |
| 20 | Ubiq-Firefly_Ubiq-Renilla-dCasRx-U6-3: | Ubiq-Firefly_Ubiq-Renilla | U6-3: | dCasRx | 11.00 | 322000000 |
| | 4-gRNA-Fluc-11 | | 4-gRNA-Fluc | | | |
| 21 | Ubiq-Firefly_Ubiq-Renilla-dCasRx-U6-3: | Ubiq-Firefly_Ubiq-Renilla | U6-3: | dCasRx | 12.00 | 345000000 |
| | 4-gRNA-Fluc-12 | | 4-gRNA-Fluc | | | |
| 22 | Ubiq-Firefly_Ubiq-Renilla-NA-1 | Ubiq-Firefly_Ubiq-Renilla | N/A | NA | 1.00 | 89232136 |
| 23 | NA-NA-U6-3: 4-gRNA-Fluc-1 | NA | U6-3: | NA | 1.00 | 365 |
| | | | 4-gRNA-Fluc | | | |
| 24 | NA-NA-U6-3: 4-gRNA-Fluc-2 | NA | U6-3: | NA | 2.00 | 1751 |
| | | | 4-gRNA-Fluc | | | |
| 25 | Ubiq-Firefly_Ubiq-Renilla-CasRx-1 | Ubiq-Firefly_Ubiq-Renilla | N/A | CasRx | 1.00 | 93210008 |
| 26 | Ubiq-Firefly_Ubiq-Renilla-CasRx-2 | Ubiq-Firefly_Ubiq-Renilla | N/A | CasRx | 2.00 | 94663808 |
| 27 | Ubiq-Firefly_Ubiq-Renilla-CasRx-3 | Ubiq-Firefly_Ubiq-Renilla | N/A | CasRx | 3.00 | 89198952 |
| 28 | Ubiq-Firefly_Ubiq-Renilla-CasRx-4 | Ubiq-Firefly_Ubiq-Renilla | N/A | CasRx | 4.00 | 73215984 |
| 29 | Ubiq-Firefly_Ubiq-Renilla-CasRx-5 | Ubiq-Firefly_Ubiq-Renilla | N/A | CasRx | 5.00 | 95205496 |
| 30 | Ubiq-Firefly_Ubiq-Renilla-CasRx-6 | Ubiq-Firefly_Ubiq-Renilla | N/A | CasRx | 6.00 | 70915592 |
| 31 | Ubiq-Firefly_Ubiq-Renilla-CasRx-7 | Ubiq-Firefly_Ubiq-Renilla | N/A | CasRx | 7.00 | 159000000 |
| 32 | Ubiq-Firefly_Ubiq-Renilla-CasRx-8 | Ubiq-Firefly_Ubiq-Renilla | N/A | CasRx | 8.00 | 218000000 |
| 33 | Ubiq-Firefly_Ubiq-Renilla-CasRx-9 | Ubiq-Firefly_Ubiq-Renilla | N/A | CasRx | 9.00 | 205000000 |
| 34 | Ubiq-Firefly_Ubiq-Renilla-CasRx-10 | Ubiq-Firefly_Ubiq-Renilla | N/A | CasRx | 10.00 | 122000000 |
| 35 | Ubiq-Firefly_Ubiq-Renilla-CasRx-11 | Ubiq-Firefly_Ubiq-Renilla | N/A | CasRx | 11.00 | 177000000 |
| 36 | Ubiq-Firefly_Ubiq-Renilla-CasRx-12 | Ubiq-Firefly_Ubiq-Renilla | N/A | CasRx | 12.00 | 131000000 |
| 37 | Ubiq-Firefly_Ubiq-Renilla-CasRx-13 | Ubiq-Firefly_Ubiq-Renilla | N/A | CasRx | 13.00 | 55582832 |
| 38 | Ubiq-Firefly_Ubiq-Renilla-CasRx-14 | Ubiq-Firefly_Ubiq-Renilla | N/A | CasRx | 14.00 | 122000000 |
| 39 | Ubiq-Firefly_Ubiq-Renilla-CasRx-15 | Ubiq-Firefly_Ubiq-Renilla | N/A | CasRx | 15.00 | 79623680 |
| 40 | Ubiq-Firefly_Ubiq-Renilla-CasRx-16 | Ubiq-Firefly_Ubiq-Renilla | N/A | CasRx | 16.00 | 172000000 |
| 41 | Ubiq-Firefly_Ubiq-Renilla-CasRx-17 | Ubiq-Firefly_Ubiq-Renilla | N/A | CasRx | 17.00 | 145000000 |

| | Column Number | | | | | |
|---|---|---|---|---|---|---|
| Row | 7 | 8 | 9 | 10 | 11 | 12 |
| Number | Renilla | Category_Name | Firefly.log | Renilla.log | Ratio | Log.Ratio |
| 1 | 1142209920 | Ubiq-Firefly_Ubiq-Renilla-dCasRx | 19.04 | 20.86 | 0.16 | 0.91 |
| 2 | 944220352 | Ubiq-Firefly_Ubiq-Renilla-dCasRx | 18.64 | 20.67 | 0.13 | 0.90 |
| 3 | 1220324224 | Ubiq-Firefly_Ubiq-Renilla-dCasRx | 19.09 | 20.92 | 0.16 | 0.91 |
| 4 | 1660237568 | Ubiq-Firefly_Ubiq-Renilla-dCasRx | 19.50 | 21.23 | 0.18 | 0.92 |
| 5 | 1847885568 | Ubiq-Firefly_Ubiq-Renilla-dCasRx | 19.63 | 21.34 | 0.18 | 0.92 |
| 6 | 2119028000 | Ubiq-Firefly_Ubiq-Renilla-dCasRx | 19.73 | 21.47 | 0.18 | 0.92 |
| 7 | 1172879616 | Ubiq-Firefly_Ubiq-Renilla-dCasRx | 19.05 | 20.88 | 0.16 | 0.91 |
| 8 | 1183900544 | Ubiq-Firefly_Ubiq-Renilla-dCasRx | 19.03 | 20.89 | 0.16 | 0.91 |
| 9 | 1343926784 | Ubiq-Firefly_Ubiq-Renilla-dCasRx | 19.22 | 21.02 | 0.16 | 0.91 |
| 10 | 1987836416 | Ubiq-Firefly_Ubiq-Renilla-dCasRx-U6-3: 4-gRNA-Fluc | 19.50 | 21.41 | 0.15 | 0.91 |
| 11 | 1559219072 | Ubiq-Firefly_Ubiq-Renilla-dCasRx-U6-3: 4-gRNA-Fluc | 19.38 | 21.17 | 0.17 | 0.92 |
| 12 | 1746218496 | Ubiq-Firefly_Ubiq-Renilla-dCasRx-U6-3: 4-gRNA-Fluc | 19.69 | 21.28 | 0.20 | 0.93 |
| 13 | 1985210240 | Ubiq-Firefly_Ubiq-Renilla-dCasRx-U6-3: 4-gRNA-Fluc | 19.76 | 21.41 | 0.19 | 0.92 |
| 14 | 2066989280 | Ubiq-Firefly_Ubiq-Renilla-dCasRx-U6-3: 4-gRNA-Fluc | 19.84 | 21.45 | 0.20 | 0.92 |
| 15 | 2190655520 | Ubiq-Firefly_Ubiq-Renilla-dCasRx-U6-3: 4-gRNA-Fluc | 19.69 | 21.51 | 0.16 | 0.92 |
| 16 | 1708601856 | Ubiq-Firefly_Ubiq-Renilla-dCasRx-U6-3: 4-gRNA-Fluc | 19.53 | 21.26 | 0.18 | 0.92 |
| 17 | 1915715968 | Ubiq-Firefly_Ubiq-Renilla-dCasRx-U6-3: 4-gRNA-Fluc | 19.55 | 21.37 | 0.16 | 0.91 |
| 18 | 1580265344 | Ubiq-Firefly_Ubiq-Renilla-dCasRx-U6-3: 4-gRNA-Fluc | 19.44 | 21.18 | 0.18 | 0.92 |
| 19 | 2123975200 | Ubiq-Firefly_Ubiq-Renilla-dCasRx-U6-3: 4-gRNA-Fluc | 19.73 | 21.48 | 0.18 | 0.92 |
| 20 | 1943790976 | Ubiq-Firefly_Ubiq-Renilla-dCasRx-U6-3: 4-gRNA-Fluc | 19.59 | 21.39 | 0.17 | 0.92 |
| 21 | 2241432640 | Ubiq-Firefly_Ubiq-Renilla-dCasRx-U6-3: 4-gRNA-Fluc | 19.66 | 21.53 | 0.15 | 0.91 |
| 22 | 1881228672 | Ubiq-Firefly_Ubiq-Renilla-NA | 18.31 | 21.36 | 0.05 | 0.86 |
| 23 | 4102 | NA-NA-U6-3: 4-gRNA-Fluc | 5.90 | 8.32 | 0.09 | 0.71 |
| 24 | 27634 | NA-NA-U6-3: 4-gRNA-Fluc | 7.47 | 10.23 | 0.06 | 0.73 |
| 25 | 1527155456 | Ubiq-Firefly_Ubiq-Renilla-CasRx | 18.35 | 21.15 | 0.06 | 0.87 |
| 26 | 1254850560 | Ubiq-Firefly_Ubiq-Renilla-CasRx | 18.37 | 20.95 | 0.08 | 0.88 |
| 27 | 1709635456 | Ubiq-Firefly_Ubiq-Renilla-CasRx | 18.31 | 21.26 | 0.05 | 0.86 |
| 28 | 1871131392 | Ubiq-Firefly_Ubiq-Renilla-CasRx | 18.11 | 21.35 | 0.04 | 0.85 |
| 29 | 1518919936 | Ubiq-Firefly_Ubiq-Renilla-CasRx | 18.37 | 21.14 | 0.06 | 0.87 |
| 30 | 1829941120 | Ubiq-Firefly_Ubiq-Renilla-CasRx | 18.08 | 21.33 | 0.04 | 0.85 |
| 31 | 1253901184 | Ubiq-Firefly_Ubiq-Renilla-CasRx | 18.88 | 20.95 | 0.13 | 0.90 |
| 32 | 1587820416 | Ubiq-Firefly_Ubiq-Renilla-CasRx | 19.20 | 21.19 | 0.14 | 0.91 |
| 33 | 1795636864 | Ubiq-Firefly_Ubiq-Renilla-CasRx | 19.14 | 21.31 | 0.11 | 0.90 |
| 34 | 1114623616 | Ubiq-Firefly_Ubiq-Renilla-CasRx | 18.62 | 20.83 | 0.11 | 0.89 |
| 35 | 1845966208 | Ubiq-Firefly_Ubiq-Renilla-CasRx | 18.99 | 21.34 | 0.10 | 0.89 |
| 36 | 2119864320 | Ubiq-Firefly_Ubiq-Renilla-CasRx | 18.69 | 21.47 | 0.06 | 0.87 |
| 37 | 2317485280 | Ubiq-Firefly_Ubiq-Renilla-CasRx | 17.83 | 21.56 | 0.02 | 0.83 |
| 38 | 1094934144 | Ubiq-Firefly_Ubiq-Renilla-CasRx | 18.62 | 20.81 | 0.11 | 0.89 |
| 39 | 1090508032 | Ubiq-Firefly_Ubiq-Renilla-CasRx | 18.19 | 20.81 | 0.07 | 0.87 |
| 40 | 1503190016 | Ubiq-Firefly_Ubiq-Renilla-CasRx | 18.97 | 21.13 | 0.11 | 0.90 |
| 41 | 1152661248 | Ubiq-Firefly_Ubiq-Renilla-CasRx | 18.80 | 20.87 | 0.13 | 0.90 |

TABLE 5
Samples for Illumina RNA Sequencing

| Name | Genotype | Related Vector Name (s) | Sample Type |
|---|---|---|---|
| CasRx_w_adult_heads-R1 | Ubiq-CasRx/+; gRNA(w)/+ | OA-1050E.2/OA-1050G | Experimental |
| dCasRx_w_adult_heads-R1 | Ubiq-dCasRx/+; gRNA(w)/+ | OA-1050R/OA-1050G | Control |
| CasRx_w_adult_heads-R2 | Ubiq-CasRx/+; gRNA(w)/+ | OA-1050E.2/OA-1050G | Experimental |
| dCasRx_w_adult_heads-R2 | Ubiq-dCasRx/+; gRNA(w)/+ | OA-1050R/OA-1050G | Control |
| CasRx_w_adult_heads-R3 | Ubiq-CasRx/+; gRNA(w)/+ | OA-1050E.2/OA-1050G | Experimental |
| dCasRx_w_adult_heads-R3 | Ubiq-dCasRx/+; gRNA(w)/+ | OA-1050R/OA-1050G | Control |
| CasRx_N_larvae-R1 | Ubiq-CasRx/+; gRNA(N)/+ | OA-1050E.2/OA-1050I | Experimental |
| dCasRx_N_larvae-R1 | Ubiq-dCasRx/+; gRNA(N)/+ | OA-1050R/OA-1050I | Control |
| CasRx_N_larvae-R2 | Ubiq-CasRx/+; gRNA(N)/+ | OA-1050E.2/OA-1050I | Experimental |
| dCasRx_N_larvae-R2 | Ubiq-dCasRx/+; gRNA(N)/+ | OA-1050R/OA-1050I | Control |
| CasRx_N_larvae-R3 | Ubiq-CasRx/+; gRNA(N)/+ | OA-1050E.2/OA-1050I | Experimental |
| dCasRx_N_larvae-R3 | Ubiq-dCasRx/+; gRNA(N)/+ | OA-1050R/OA-1050I | Control |
| CasRx_GFP_larvae-R1 | Ubiq-CasRx/+; gRNA(GFP)/+ | OA-1050E.2/OA-1050J | Experimental |
| dCasRx_GFP_larvae-R1 | Ubiq-dCasRx/+; gRNA(GFP)/+ | OA-1050R/OA-1050J | Control |
| CasRx_GFP_larvae-R2 | Ubiq-CasRx/+; gRNA(GFP)/+ | OA-1050E.2/OA-1050J | Experimental |
| dCasRx_GFP_larvae-R2 | Ubiq-dCasRx/+; gRNA(GFP)/+ | OA-1050R/OA-1050J | Control |
| CasRx_GFP_larvae-R3 | Ubiq-CasRx/+; gRNA(GFP)/+ | OA-1050E.2/OA-1050J | Experimental |
| dCasRx_GFP_larvae-R3 | Ubiq-dCasRx/+; gRNA(GFP)/+ | OA-1050R/OA-1050J | Control |
| CasRx_cn_embryos-R1 | Ubiq-CasRx/+; gRNA(cn)/+ & CyO/+; gRNA(cn)/+ | OA-1050E.2/OA-1050U | Experimental |
| dCasRx_cn_embryos-R1 | Ubiq-dCasRx/+; gRNA(cn)/+ & CyO/+; gRNA(cn)/+ | OA-1050R/OA-1050U | Control |
| CasRx_cn_embryos-R2 | Ubiq-CasRx/+; gRNA(cn)/+ & CyO/+; gRNA(cn)/+ | OA-1050E.2/OA-1050U | Experimental |
| dCasRx_cn_embryos-R2 | Ubiq-dCasRx/+; gRNA(cn)/+ & CyO/+; gRNA(cn)/+ | OA-1050R/OA-1050U | Control |
| CasRx_wg_embryos-R1 | Ubiq-CasRx/+; gRNA(wg)/+ & CyO/+; gRNA(wg)/+ | OA-1050E.2/OA-1050V | Experimental |
| dCasRx_wg_embryos-R1 | Ubiq-dCasRx/+; gRNA(wg)/+ & CyO/+; gRNA(wg)/+ | OA-1050R/OA-1050V | Control |
| CasRx_wg_embryos-R2 | Ubiq-CasRx/+; gRNA(wg)/+ & CyO/+; gRNA(wg)/+ | OA-1050E.2/OA-1050V | Experimental |
| dCasRx_wg_embryos-R2 | Ubiq-dCasRx/+; gRNA(wg)/+ & CyO/+; gRNA(wg)/+ | OA-1050R/OA-1050V | Control |
| CasRx_wg_embryos-R3 | Ubiq-CasRx/+; gRNA(wg)/+ & CyO/+; gRNA(wg)/+ | OA-1050E.2/OA-1050V | Experimental |
| dCasRx_wg_embryos-R3 | Ubiq-dCasRx/+; gRNA(wg)/+ & CyO/+; gRNA(wg)/+ | OA-1050R/OA-1050V | Control |
| CasRx_y_embryos-R1 | Ubiq-CasRx/+; gRNA(y)/+ & CyO/+; gRNA(y)/+ | OA-1050E.2/OA-1050Z4 | Experimental |
| dCasRx_y_embryos-R1 | Ubiq-dCasRx/+; gRNA(y)/+ & CyO/+; gRNA(y)/+ | OA-1050R/OA-1050Z4 | Control |
| CasRx_y_embryos-R2 | Ubiq-CasRx/+; gRNA(y)/+ & CyO/+; gRNA(y)/+ | OA-1050E.2/OA-1050Z4 | Experimental |
| dCasRx_y_embryos-R2 | Ubiq-dCasRx/+; gRNA(y)/+ & CyO/+; gRNA(y)/+ | OA-1050R/OA-1050Z4 | Control |
| CasRx_y_embryos-R3 | Ubiq-CasRx/+; gRNA(y)/+ & CyO/+; gRNA(y)/+ | OA-1050E.2/OA-1050Z4 | Experimental |
| dCasRx_y_embryos-R3 | Ubiq-dCasRx/+; gRNA(y)/+ & CyO/+; gRNA(y)/+ | OA-1050R/OA-1050Z4 | Control |

TABLE 6
All TPM data

| | | | | 22067. | 22068. | 22069. |
|---|---|---|---|---|---|---|
| | | 22065. | 22066. | CasRx_{—} | dCasRx_{—} | CasRx_{—} |
| | | CasRx_{—} | dCasRx_{—} | N_{—} | N_{—} | GFP_{—} |
| | | w_adults_{—} | w_adults_{—} | larvae_{—} | larvae_{—} | larvae_{—} |
| Row | | Dmel_{—} | Dmel_{—} | Dmel_{—} | Dmel_{—} | Dmel_{—} |
| Number | ID | STAR | STAR | STAR | STAR | STAR |
| 1 | CasRx | 147.8780225 | 168.1281395 | 87.43139822 | 129.9983832 | 456.9116063 |
| 2 | GFP | 40.82274848 | 17.03460786 | 252.0574783 | 188.7277072 | 297.3345117 |
| 3 | GFP_target_1 | 0 | 0 | 0 | 0 | 79.87279381 |
| 4 | GFP_target_2 | 0.328936481 | 0 | 0 | 0 | 88.09499317 |
| 5 | GFP_target_3 | 0.328936481 | 0 | 0 | 0 | 55.20619572 |
| 6 | GFP_target_4 | 0.328936481 | 0 | 0 | 0 | 2.349199818 |
| 7 | cinnabar_target_1 | 0 | 0 | 0 | 0 | 0 |
| 8 | cinnabar_target_2 | 0 | 0 | 0 | 0 | 0 |
| 9 | cinnabar_target_3 | 0 | 0 | 0 | 0 | 0 |
| 10 | cinnabar_target_4 | 0 | 0 | 0 | 0 | 0 |
| 11 | notch_target_1 | 0 | 0 | 60.155027 | 65.05186067 | 0 |
| 12 | notch_target_2 | 0 | 0 | 22.55813512 | 25.18136542 | 0 |
| 13 | notch_target_3 | 0 | 0 | 13.15891216 | 20.98447118 | 0 |
| 14 | notch_target_4 | 0.328936481 | 0 | 0 | 0 | 0 |
| 15 | white_target_1 | 13.4863957 | 16.15057632 | 0 | 0 | 0 |
| 16 | white_target_2 | 57.56388409 | 35.5312679 | 0 | 0 | 0 |
| 17 | white_target_3 | 23.35449012 | 16.15057632 | 0 | 0 | 0 |
| 18 | white_target_4 | 27.95960084 | 3.230115264 | 0 | 0 | 0 |
| 19 | wingless_target_1 | 0.328936481 | 0 | 0 | 0 | 0 |
| 20 | wingless_target_2 | 0.328936481 | 0 | 0 | 0 | 0 |
| 21 | wingless_target_3 | 0 | 0 | 0 | 0 | 0 |
| 22 | wingless_target_4 | 0.328936481 | 0 | 0 | 0 | 0 |
| 23 | y_target_1 | 0 | 0 | 0 | 0 | 0 |
| 24 | y_target_2 | 0 | 0 | 0 | 0 | 0 |
| 25 | y_target_3 | 0.328936481 | 0 | 0 | 0 | 0 |
| 26 | y_target_4 | 0 | 0 | 0 | 0 | 0 |

| | 22070. | 22071. | 22072. | 22104. | 22105. | 22106. |
|---|---|---|---|---|---|---|
| | dCasRx_{—} | CasRx_{—} | dCasRx_{—} | CasRx_{—} | dCasRx_{—} | CasRx_{—} |
| | GFP_{—} | cn_{—} | cn_{—} | wg_{—} | wg_{—} | y_{—} |
| | larvae_{—} | embryos_{—} | embryos_{—} | embryos_{—} | embryos_{—} | embryos_{—} |
| Row | Dmel_{—} | Dmel_{—} | Dmel_{—} | Dmel_{—} | Dmel_{—} | Dmel_{—} |
| Number | STAR | STAR | STAR | STAR | STAR | STAR |
| 1 | 209.3030314 | 228.1725621 | 165.2057063 | 142.7686257 | 242.2031821 | 67.53354135 |
| 2 | 671.8565481 | 18.36587656 | 17.84618193 | 12.96483796 | 19.39454814 | 24.12459907 |
| 3 | 25.49867548 | 0 | 0 | 0 | 0 | 0 |
| 4 | 69.05891277 | 0 | 0 | 0 | 0 | 0 |
| 5 | 57.37201984 | 0 | 0 | 0 | 0 | 0 |
| 6 | 3.187334435 | 0 | 0 | 0 | 0 | 0 |
| 7 | 0 | 8.879176964 | 0 | 0 | 0 | 0 |
| 8 | 0 | 11.83890262 | 5.526934908 | 0 | 0 | 0 |
| 9 | 0 | 23.67780524 | 27.63467454 | 0 | 0 | 0 |
| 10 | 0 | 0 | 0 | 0 | 0 | 0 |
| 11 | 0 | 0 | 0 | 0 | 0 | 0 |
| 12 | 0 | 0 | 0 | 0 | 0 | 0 |
| 13 | 0 | 0 | 0 | 0 | 0 | 0 |
| 14 | 0 | 0 | 0 | 0 | 0 | 0 |
| 15 | 0 | 0 | 0 | 0 | 0 | 0 |
| 16 | 0 | 0 | 0 | 0 | 0 | 0 |
| 17 | 0 | 0 | 0 | 0 | 0 | 0 |
| 18 | 0 | 0 | 0 | 0 | 0 | 0 |
| 19 | 0 | 0 | 0 | 0 | 0 | 0 |
| 20 | 0 | 0 | 0 | 3.041134831 | 2.779007652 | 0 |
| 21 | 0 | 0 | 0 | 0 | 0 | 0 |
| 22 | 0 | 0 | 0 | 0 | 2.779007652 | 0 |
| 23 | 0 | 0 | 0 | 0 | 0 | 13.1714765 |
| 24 | 0 | 0 | 0 | 0 | 0 | 7.317486946 |
| 25 | 0 | 0 | 0 | 0 | 0 | 16.09847128 |
| 26 | 0 | 0 | 0 | 0 | 0 | 1.463497389 |

| | 22107. | 22225. | 22226. | 22227. | 22228. | 22229. |
|---|---|---|---|---|---|---|
| | dCasRx_{—} | CasRx_{—} | dCasRx_{—} | CasRx_{—} | dCasRx_{—} | CasRx_{—} |
| | y_{—} | w_{—} | w_{—} | N_{—} | N | GFP_{—} |
| | embryos_{—} | adultsR2_{—} | adultsR2_{—} | larvaeR2_{—} | larvaeR2_{—} | larvaeR2_{—} |
| Row | Dmel_{—} | Dmel_{—} | Dmel_{—} | Dmel_{—} | Dmel_{—} | Dmel_{—} |
| Number | STAR | STAR | STAR | STAR | STAR | STAR |
| 1 | 25.83680679 | 231.0873809 | 107.7509761 | 181.0369279 | 156.4167661 | 347.2688606 |
| 2 | 11.26778644 | 19.54894009 | 17.64518464 | 271.8726424 | 222.3475142 | 126.3222603 |
| 3 | 0 | 0 | 0 | 0 | 0 | 74.1637063 |
| 4 | 0.254866598 | 0 | 0 | 0 | 0 | 60.25801137 |
| 5 | 0.254866598 | 0 | 0 | 0 | 0 | 66.43832023 |
| 6 | 0.254866598 | 0 | 0 | 0 | 0 | 0 |
| 7 | 0 | 0 | 0 | 0 | 0 | 0 |
| 8 | 0 | 0 | 0 | 0 | 0 | 0 |
| 9 | 0 | 0 | 0 | 0 | 0 | 0 |
| 10 | 0 | 0 | 0 | 0 | 0 | 0 |
| 11 | 0 | 0 | 0 | 111.586146 | 112.6376841 | 0 |
| 12 | 0 | 0 | 0 | 47.20952332 | 45.97456492 | 0 |
| 13 | 0 | 0 | 0 | 32.18831135 | 34.48092369 | 0 |
| 14 | 0.254866598 | 0 | 0 | 2.145887423 | 2.298728246 | 0 |
| 15 | 0.254866598 | 11.46388462 | 9.061040759 | 0 | 0 | 0 |
| 16 | 0 | 20.06179808 | 10.57121422 | 0 | 0 | 0 |
| 17 | 0.254866598 | 17.19582693 | 7.550867299 | 0 | 0 | 0 |
| 18 | 0 | 20.06179808 | 21.14242844 | 0 | 0 | 0 |
| 19 | 0.254866598 | 0 | 0 | 0 | 0 | 0 |
| 20 | 0.254866598 | 0 | 0 | 0 | 0 | 0 |
| 21 | 0 | 0 | 0 | 0 | 0 | 0 |
| 22 | 0.254866598 | 0 | 0 | 0 | 0 | 0 |
| 23 | 7.645997943 | 0 | 0 | 0 | 0 | 0 |
| 24 | 0 | 0 | 0 | 0 | 0 | 0 |
| 25 | 10.44953052 | 0 | 0 | 0 | 0 | 0 |
| 26 | 0 | 0 | 0 | 0 | 0 | 0 |

| | | 22230. | 22231. | 22232. | 22233. | 22234. |
|---|---|---|---|---|---|---|
| | | dCasRx_{—} | CasRx_{—} | dCasRx_{—} | CasRx_{—} | dCasRx_{—} |
| | | GFP_{—} | cn_{—} | cn_{—} | wg_{—} | wg_{—} |
| | | larvaeR2_{—} | embryos_{—} | embryos_{—} | embryosR2 | embryosR2_{—} |
| Row | | Dmel_{—} | Dmel_{—} | Dmel_{—} | Dmel_{—} | Dmel_{—} |
| Number | ID | STAR | STAR | STAR | STAR | STAR |
| 1 | CasRx | 279.4293241 | 296.7059376 | 225.2311743 | 392.7726329 | 465.3581939 |
| 2 | GFF | 704.34024 | 15.59954876 | 17.11425909 | 4.344017484 | 6.270862507 |
| 3 | GFP_target_1 | 68.34649687 | 0 | 0 | 0 | 0 |
| 4 | GFP_target_2 | 73.85831114 | 0 | 0 | 0 | 0 |
| 5 | GFP_target_3 | 38.58269985 | 0 | 0 | 0 | 0 |
| 6 | GFP_target_4 | 11.02362853 | 0 | 0 | 0 | 0 |
| 7 | cinnabar_target_1 | 0 | 6.478501124 | 23.37257306 | 0 | 0 |
| 8 | cinnabar_target_2 | 0 | 19.43550337 | 32.13728796 | 0 | 0 |
| 9 | cinnabar_target_3 | 0 | 22.67475394 | 43.82357449 | 0 | 0 |
| 10 | cinnabar_target_4 | 0 | 0 | 2.921571633 | 0 | 0 |
| 11 | notch_target_1 | 0 | 0 | 0 | 0 | 0 |
| 12 | notch_target_2 | 0 | 0 | 0 | 0 | 0 |
| 13 | notch_target_3 | 0 | 0 | 0 | 0 | 0 |
| 14 | notch_target_4 | 0 | 0 | 0 | 0 | 0 |
| 15 | white_target_1 | 0 | 0 | 0 | 0 | 0 |
| 16 | white_target_2 | 0 | 0 | 0 | 0 | 0 |
| 17 | white_target_3 | 0 | 0 | 0 | 0 | 0 |
| 18 | white_target_4 | 0 | 0 | 0 | 0 | 0 |
| 19 | wingless_target_1 | 0 | 0 | 0 | 2.751211073 | 6.304041674 |
| 20 | wingless_target_2 | 0 | 0 | 0 | 2.751211073 | 6.304041674 |
| 21 | wingless_target_3 | 0 | 0 | 0 | 2.751211073 | 15.76010418 |
| 22 | wingless_target_4 | 0 | 0 | 0 | 2.751211073 | 0 |
| 23 | y_target_1 | 0 | 0 | 0 | 0 | 0 |
| 24 | y_target_2 | 0 | 0 | 0 | 0 | 0 |
| 25 | y_target_3 | 0 | 0 | 0 | 0 | 0 |
| 26 | y_target_4 | 0 | 0 | 0 | 0 | 0 |

| | 22235. | 22236. | 22240. | 22241. | 22242. | 22243. |
|---|---|---|---|---|---|---|
| | CasRx_{—} | dCasRx_{—} | CasRx_{—} | dCasRx_{—} | CasRx_{—} | dCasRx_{—} |
| | y_{—} | y_{—} | w_{—} | w_{—} | N_{—} | N_{—} |
| | embryosR2_{—} | embryosR2_{—} | adultsR3_{—} | adultsR3_{—} | larvaeR3_{—} | larvaeR3_{—} |
| Row | Dmel_{—} | Dmel_{—} | Dmel_{—} | Dmel_{—} | Dmel_{—} | Dmel_{—} |
| Number | STAR | STAR | STAR | STAR | STAR | STAR |
| 1 | 86.0685427 | 40.44460927 | 228.4806837 | 139.8759205 | 136.3335448 | 131.5504354 |
| 2 | 39.27308486 | 16.74077096 | 12.28186452 | 28.24378489 | 220.7840576 | 248.8056154 |
| 3 | 0 | 0 | 0 | 0 | 0 | 2.559728553 |
| 4 | 0 | 0 | 0 | 0 | 0 | 0 |
| 5 | 0 | 0 | 0 | 0 | 0 | 0 |
| 6 | 0 | 0 | 0 | 0 | 0 | 0 |
| 7 | 0 | 0 | 0 | 0 | 0 | 0 |
| 8 | 0 | 0 | 0 | 0 | 0 | 0 |
| 9 | 0 | 0 | 0 | 0 | 0 | 0 |
| 10 | 0 | 0 | 0 | 0 | 0 | 0 |
| 11 | 0 | 0 | 0 | 0 | 92.18287806 | 84.47104225 |
| 12 | 0 | 0 | 0 | 0 | 30.24750686 | 35.83619974 |
| 13 | 0 | 0 | 0 | 0 | 27.36679192 | 38.3959283 |
| 14 | 0 | 0 | 0 | 0 | 2.880714939 | 5.119457106 |
| 15 | 0 | 0 | 17.57856316 | 0 | 0 | 0 |
| 16 | 0 | 0 | 57.13033027 | 23.23081874 | 0 | 0 |
| 17 | 0 | 0 | 13.18392237 | 11.61540937 | 0 | 0 |
| 18 | 0 | 0 | 8.789281581 | 3.871803123 | 0 | 0 |
| 19 | 0 | 0 | 0 | 0 | 0 | 0 |
| 20 | 0 | 0 | 0 | 0 | 0 | 0 |
| 21 | 0 | 0 | 0 | 0 | 0 | 0 |
| 22 | 0 | 0 | 0 | 0 | 0 | 0 |
| 23 | 7.645375126 | 7.537313939 | 0 | 0 | 0 | 0 |
| 24 | 7.645375126 | 5.024875959 | 0 | 0 | 0 | 0 |
| 25 | 26.75881294 | 18.84328485 | 0 | 0 | 0 | 0 |
| 26 | 6.371145938 | 6.281094949 | 0 | 0 | 0 | 0 |

| | 22244. | 22245. | 22248. | 22249. | 22250. | 22251. |
|---|---|---|---|---|---|---|
| | CasRx_{—} | dCasRx_{—} | CasRx_{—} | dCasRx_{—} | CasRx_{—} | dCasRx_{—} |
| | GF_{—} | GFP_{—} | wg_{—} | wg_{—} | y_{—} | y_{—} |
| | larvaeR3_{—} | larvaeR3_{—} | embryosR3_{—} | embryosR3_{—} | embryosR3_{—} | embryosR3_{—} |
| Row | Dmel_{—} | Dmel_{—} | Dmel_{—} | Dmel_{—} | Dmel_{—} | Dmel_{—} |
| Number | STAR | STAR | STAR | STAR | STAR | STAR |
| 1 | 452.7981069 | 257.1240248 | 322.4951807 | 400.7910187 | 72.48190139 | 47.08655474 |
| 2 | 263.4128981 | 645.6344105 | 8.103968092 | 7.614372662 | 84.47293343 | 20.91309311 |
| 3 | 95.51710734 | 74.95700022 | 0 | 0 | 0 | 0 |
| 4 | 85.46267499 | 95.39981846 | 0 | 0 | 0 | 0 |
| 5 | 87.97628308 | 40.88563648 | 0 | 0 | 0 | 0 |
| 6 | 0 | 6.814272747 | 0 | 0 | 0 | 0 |
| 7 | 0 | 0 | 0 | 0 | 0 | 0 |
| 8 | 0 | 0 | 0 | 0 | 0 | 0 |
| 9 | 0 | 0 | 0 | 0 | 0 | 0 |
| 10 | 0 | 0 | 0 | 0 | 0 | 0 |
| 11 | 0 | 0 | 0 | 0 | 0 | 0 |
| 12 | 0 | 0 | 0 | 0 | 0 | 0 |
| 13 | 0 | 0 | 0 | 0 | 0 | 0 |
| 14 | 0 | 0 | 0 | 0 | 0 | 0 |
| 15 | 0 | 0 | 0 | 0 | 0 | 0 |
| 16 | 0 | 0 | 0 | 0 | 0 | 0 |
| 17 | 0 | 0 | 0 | 0 | 0 | 0 |
| 18 | 0 | 0 | 0 | 0 | 0 | 0 |
| 19 | 0 | 0 | 5.76686868 | 3.071615299 | 0 | 0 |
| 20 | 0 | 0 | 14.4171717 | 6.143230598 | 0 | 0 |
| 21 | 0 | 0 | 28.8343434 | 30.71615299 | 0 | 0 |
| 22 | 0 | 0 | 0 | 0 | 0 | 0 |
| 23 | 0 | 0 | 0 | 0 | 2.445133661 | 4.86947021 |
| 24 | 0 | 0 | 0 | 0 | 14.67080197 | 0 |
| 25 | 0 | 0 | 0 | 0 | 17.11593563 | 10.95630797 |
| 26 | 0 | 0 | 0 | 0 | 4.890267322 | 1.217367552 |

TABLE 7
All Count Data

| | | 22065. | 22066. | 22067. | 22068. | 22069. |
|---|---|---|---|---|---|---|
| | | CasRx_{—} | dCasRx_{—} | CasRx_{—} | dCasRx_{—} | CasRx_{—} |
| | | w_{—} | w_{—} | N_{—} | N_{—} | GFP_{—} |
| | | adults_{—} | adults_{—} | larvae_{—} | larvae_{—} | larvae_{—} |
| Row | | Dmel_{—} | Dmel_{—} | Dmel_{—} | Dmel_{—} | Dmel_{—} |
| Number | ID | STAR | STAR | STAR | STAR | STAR |
| 1 | CasRx | 5672 | 6567 | 5868 | 7816 | 24539 |
| 2 | GFP | 393 | 167 | 4246 | 2848 | 4008 |
| 3 | GFP_target_1 | 0 | 0 | 0 | 0 | 34 |
| 4 | GFP_target_2 | 0.1 | 0 | 0 | 0 | 37.5 |
| 5 | GFP_target_3 | 0.1 | 0 | 0 | 0 | 23.5 |
| 6 | GFP_target_4 | 0.1 | 0 | 0 | 0 | 1 |
| 7 | cinnabar_target_1 | 0 | 0 | 0 | 0 | 0 |
| 8 | cinnabar_target_2 | 0 | 0 | 0 | 0 | 0 |
| 9 | cinnabar_target_3 | 0 | 0 | 0 | 0 | 0 |
| 10 | cinnabar_target_4 | 0 | 0 | 0 | 0 | 0 |
| 11 | notch_target_1 | 0 | 0 | 32 | 31 | 0 |
| 12 | notch_target_2 | 0 | 0 | 12 | 12 | 0 |
| 13 | notch_target_3 | 0 | 0 | 7 | 10 | 0 |
| 14 | notch_target_4 | 0.1 | 0 | 0 | 0 | 0 |
| 15 | white_target_1 | 4.1 | 5 | 0 | 0 | 0 |
| 16 | white_target_2 | 17.5 | 11 | 0 | 0 | 0 |
| 17 | white_target_3 | 7.1 | 5 | 0 | 0 | 0 |
| 18 | white_target_4 | 8.5 | 1 | 0 | 0 | 0 |
| 19 | wingless_target_1 | 0.1 | 0 | 0 | 0 | 0 |
| 20 | wingless_target_2 | 0.1 | 0 | 0 | 0 | 0 |
| 21 | wingless_target_3 | 0 | 0 | 0 | 0 | 0 |
| 22 | wingless_target_4 | 0.1 | 0 | 0 | 0 | 0 |
| 23 | y_target_1 | 0 | 0 | 0 | 0 | 0 |
| 24 | y_target_2 | 0 | 0 | 0 | 0 | 0 |
| 25 | y_target_3 | 0.1 | 0 | 0 | 0 | 0 |
| 26 | y_target_4 | 0 | 0 | 0 | 0 | 0 |

| | 22070. | 22071. | 22072. | 22104. | 22105. | 22106. |
|---|---|---|---|---|---|---|
| | dCasRx_{—} | CasRx_{—} | dCasRx_{—} | CasRx_{—} | dCasRx_{—} | CasRx_{—} |
| | GFP_{—} | cn_{—} | cn_{—} | wg_{—} | wg_{—} | y_{—} |
| | larvae_{—} | embryos_{—} | embryos_{—} | embryos_{—} | embryos_{—} | embryos_{—} |
| Row | Dme1_{—} | Dmel_{—} | Dmel_{—} | Dmel_{—} | Dme1_{—} | Dmel_{—} |
| Number | STAR | STAR | STAR | STAR | STAR | STAR |
| 1 | 12427.5 | 9726.5 | 7542.5 | 5923 | 10996 | 2911 |
| 2 | 10012.5 | 196.5 | 204.5 | 135 | 221 | 261 |
| 3 | 12 | 0 | 0 | 0 | 0 | 0 |
| 4 | 32.5 | 0 | 0 | 0 | 0 | 0 |
| 5 | 27 | 0 | 0 | 0 | 0 | 0 |
| 6 | 1.5 | 0 | 0 | 0 | 0 | 0 |
| 7 | 0 | 3 | 0 | 0 | 0 | 0 |
| 8 | 0 | 4 | 2 | 0 | 0 | 0 |
| 9 | 0 | 8 | 10 | 0 | 0 | 0 |
| 10 | 0 | 0 | 0 | 0 | 0 | 0 |
| 11 | 0 | 0 | 0 | 0 | 0 | 0 |
| 12 | 0 | 0 | 0 | 0 | 0 | 0 |
| 13 | 0 | 0 | 0 | 0 | 0 | 0 |
| 14 | 0 | 0 | 0 | 0 | 0 | 0 |
| 15 | 0 | 0 | 0 | 0 | 0 | 0 |
| 16 | 0 | 0 | 0 | 0 | 0 | 0 |
| 17 | 0 | 0 | 0 | 0 | 0 | 0 |
| 18 | 0 | 0 | 0 | 0 | 0 | 0 |
| 19 | 0 | 0 | 0 | 0 | 0 | 0 |
| 20 | 0 | 0 | 0 | 1 | 1 | 0 |
| 21 | 0 | 0 | 0 | 0 | 0 | 0 |
| 22 | 0 | 0 | 0 | 0 | 1 | 0 |
| 23 | 0 | 0 | 0 | 0 | 0 | 4.5 |
| 24 | 0 | 0 | 0 | 0 | 0 | 2.5 |
| 25 | 0 | 0 | 0 | 0 | 0 | 5.5 |
| 26 | 0 | 0 | 0 | 0 | 0 | 0.5 |

| | 22107. | 22225. | 22226. | 22227. | 22228. | 22229. |
|---|---|---|---|---|---|---|
| | dCasRx_{—} | CasRx_{—} | dCasRx_{—} | CasRx_{—} | dCasRx_{—} | CasRx_{—} |
| | y_{—} | w_{—} | w_{—} | N_{—} | N_{—} | GFP_{—} |
| | embryos_{—} | adultsR2_{—} | adultsR2_{—} | larvaeR2_{—} | larvaeR2_{—} | larvaeR2_{—} |
| Row | Dmel_{—} | Dmel_{—} | Dmel_{—} | Dmel_{—} | Dmel_{—} | Dmel_{—} |
| Number | STAR | STAR | STAR | STAR | STAR | STAR |
| 1 | 1279 | 10173 | 4501 | 10644 | 8585 | 14178.5 |
| 2 | 140 | 216 | 185 | 4012 | 3063 | 1294.5 |
| 3 | 0 | 0 | 0 | 0 | 0 | 24 |
| 4 | 0.1 | 0 | 0 | 0 | 0 | 19.5 |
| 5 | 0.1 | 0 | 0 | 0 | 0 | 21.5 |
| 6 | 0.1 | 0 | 0 | 0 | 0 | 0 |
| 7 | 0 | 0 | 0 | 0 | 0 | 0 |
| 8 | 0 | 0 | 0 | 0 | 0 | 0 |
| 9 | 0 | 0 | 0 | 0 | 0 | 0 |
| 10 | 0 | 0 | 0 | 0 | 0 | 0 |
| 11 | 0 | 0 | 0 | 52 | 49 | 0 |
| 12 | 0 | 0 | 0 | 22 | 20 | 0 |
| 13 | 0 | 0 | 0 | 15 | 15 | 0 |
| 14 | 0.1 | 0 | 0 | 1 | 1 | 0 |
| 15 | 0.1 | 4 | 3 | 0 | 0 | 0 |
| 16 | 0 | 7 | 3.5 | 0 | 0 | 0 |
| 17 | 0.1 | 6 | 2.5 | 0 | 0 | 0 |
| 18 | 0 | 7 | 7 | 0 | 0 | 0 |
| 19 | 0.1 | 0 | 0 | 0 | 0 | 0 |
| 20 | 0.1 | 0 | 0 | 0 | 0 | 0 |
| 21 | 0 | 0 | 0 | 0 | 0 | 0 |
| 22 | 0.1 | 0 | 0 | 0 | 0 | 0 |
| 23 | 3 | 0 | 0 | 0 | 0 | 0 |
| 24 | 0 | 0 | 0 | 0 | 0 | 0 |
| 25 | 4.1 | 0 | 0 | 0 | 0 | 0 |
| 26 | 0 | 0 | 0 | 0 | 0 | 0 |

| | | 22230. | 22231. | 22232. | 22233. | 22234. |
|---|---|---|---|---|---|---|
| | | dCasRx_{—} | CasRx_{—} | dCasRx_{—} | CasRx_{—} | dCasRx_{—} |
| | | GFP_{—} | cn_{—} | cn_{—} | wg_{—} | wg_{—} |
| | | larvae | embryos | embryos_{—} | embryos | embryos |
| Row | | R2_Dmel_{—} | Dmel_{—} | Dmel_{—} | R2_Dmel_{—} | R2_Dmel_{—} |
| Number | ID | STAR | STAR | STAR | STAR | STAR |
| 1 | CasRx | 15990.5 | 11556.5 | 9726.5 | 18012 | 18627 |
| 2 | GFP | 10116.5 | 152.5 | 185.5 | 50 | 63 |
| 3 | GFP_target_1 | 31 | 0 | 0 | 0 | 0 |
| 4 | GFP_target_2 | 33.5 | 0 | 0 | 0 | 0 |
| 5 | GFP_target_3 | 17.5 | 0 | 0 | 0 | 0 |
| 6 | GFP_target_4 | 5 | 0 | 0 | 0 | 0 |
| 7 | cinnabar_target_1 | 0 | 2 | 8 | 0 | 0 |
| 8 | cinnabar_target_2 | 0 | 6 | 11 | 0 | 0 |
| 9 | cinnabar_target_3 | 0 | 7 | 15 | 0 | 0 |
| 10 | cinnabar_target_4 | 0 | 0 | 1 | 0 | 0 |
| 11 | notch_target_1 | 0 | 0 | 0 | 0 | 0 |
| 12 | notch_target_2 | 0 | 0 | 0 | 0 | 0 |
| 13 | notch_target_3 | 0 | 0 | 0 | 0 | 0 |
| 14 | notch_target_4 | 0 | 0 | 0 | 0 | 0 |
| 15 | white_target_1 | 0 | 0 | 0 | 0 | 0 |
| 16 | white_target_2 | 0 | 0 | 0 | 0 | 0 |
| 17 | white_target_3 | 0 | 0 | 0 | 0 | 0 |
| 18 | white_target_4 | 0 | 0 | 0 | 0 | 0 |
| 19 | wingless_target_1 | 0 | 0 | 0 | 1 | 2 |
| 20 | wingless_target_2 | 0 | 0 | 0 | 1 | 2 |
| 21 | wingless_target_3 | 0 | 0 | 0 | 1 | 5 |
| 22 | wingless_target_4 | 0 | 0 | 0 | 1 | 0 |
| 23 | y_target_1 | 0 | 0 | 0 | 0 | 0 |
| 24 | y_target_2 | 0 | 0 | 0 | 0 | 0 |
| 25 | y_target_3 | 0 | 0 | 0 | 0 | 0 |
| 26 | y_target_4 | 0 | 0 | 0 | 0 | 0 |

| | 22235. | 22236. | 22240. | 22241. | 22242. | 22243. |
|---|---|---|---|---|---|---|
| | CasRx_{—} | dCasRx_{—} | CasRx_{—} | dCasRx_{—} | CasRx_{—} | dCasRx_{—} |
| | y_{—} | y_{—} | w_{—} | w_{—} | N_{—} | N_{—} |
| | embryosR2_{—} | embryosR2_{—} | adultsR3 | adultsR3_{—} | larvaeR3_{—} | larvaeR3_{—} |
| Row | Dmel_{—} | Dmel_{—} | Dmel-S | Dmel_{—} | Dmel_{—} | Dmel_{—} |
| Number | STAR | STAR | TAR | STAR | STAR | STAR |
| 1 | 4261 | 2031 | 6559.5 | 4558 | 5971 | 6484 |
| 2 | 488 | 211 | 88.5 | 231 | 2427 | 3078 |
| 3 | 0 | 0 | 0 | 0 | 0 | 1 |
| 4 | 0 | 0 | 0 | 0 | 0 | 0 |
| 5 | 0 | 0 | 0 | 0 | 0 | 0 |
| 6 | 0 | 0 | 0 | 0 | 0 | 0 |
| 7 | 0 | 0 | 0 | 0 | 0 | 0 |
| 8 | 0 | 0 | 0 | 0 | 0 | 0 |
| 9 | 0 | 0 | 0 | 0 | 0 | 0 |
| 10 | 0 | 0 | 0 | 0 | 0 | 0 |
| 11 | 0 | 0 | 0 | 0 | 32 | 33 |
| 12 | 0 | 0 | 0 | 0 | 10.5 | 14 |
| 13 | 0 | 0 | 0 | 0 | 9.5 | 15 |
| 14 | 0 | 0 | 0 | 0 | 1 | 2 |
| 15 | 0 | 0 | 4 | 0 | 0 | 0 |
| 16 | 0 | 0 | 13 | 6 | 0 | 0 |
| 17 | 0 | 0 | 3 | 3 | 0 | 0 |
| 18 | 0 | 0 | 2 | 1 | 0 | 0 |
| 19 | 0 | 0 | 0 | 0 | 0 | 0 |
| 20 | 0 | 0 | 0 | 0 | 0 | 0 |
| 21 | 0 | 0 | 0 | 0 | 0 | 0 |
| 22 | 0 | 0 | 0 | 0 | 0 | 0 |
| 23 | 3 | 3 | 0 | 0 | 0 | 0 |
| 24 | 3 | 2 | 0 | 0 | 0 | 0 |
| 25 | 10.5 | 7.5 | 0 | 0 | 0 | 0 |
| 26 | 2.5 | 2.5 | 0 | 0 | 0 | 0 |

| | 22244. | 22245. | 22248. | 22249. | 22250. | 22251. |
|---|---|---|---|---|---|---|
| | CasRx_{—} | dCasRx_{—} | CasRx_{—} | dCasRx_{—} | CasRx_{—} | dCasRx |
| | GFP_{—} | GFP_{—} | wg_{—} | wg_{—} | y_{—} | y_{—} |
| | larvaeR3_{—} | larvaeR3_{—} | embryosR3_{—} | embryosR3_{—} | embryosR3_{—} | embryosR3_{—} |
| Row | Dmel_{—} | Dmel_{—} | Dmel_{—} | Dmel_{—} | Dme1_{—} | Dmel_{—} |
| Number | STAR | STAR | STAR | STAR | STAR | STAR |
| 1 | 22727.5 | 14282 | 14111 | 16462.5 | 3740 | 2440 |
| 2 | 3318.5 | 9001 | 89 | 78.5 | 1094 | 272 |
| 3 | 38 | 33 | 0 | 0 | 0 | 0 |
| 4 | 34 | 42 | 0 | 0 | 0 | 0 |
| 5 | 35 | 18 | 0 | 0 | 0 | 0 |
| 6 | 0 | 3 | 0 | 0 | 0 | 0 |
| 7 | 0 | 0 | 0 | 0 | 0 | 0 |
| 8 | 0 | 0 | 0 | 0 | 0 | 0 |
| 9 | 0 | 0 | 0 | 0 | 0 | 0 |
| 10 | 0 | 0 | 0 | 0 | 0 | 0 |
| 11 | 0 | 0 | 0 | 0 | 0 | 0 |
| 12 | 0 | 0 | 0 | 0 | 0 | 0 |
| 13 | 0 | 0 | 0 | 0 | 0 | 0 |
| 14 | 0 | 0 | 0 | 0 | 0 | 0 |
| 15 | 0 | 0 | 0 | 0 | 0 | 0 |
| 16 | 0 | 0 | 0 | 0 | 0 | 0 |
| 17 | 0 | 0 | 0 | 0 | 0 | 0 |
| 18 | 0 | 0 | 0 | 0 | 0 | 0 |
| 19 | 0 | 0 | 2 | 1 | 0 | 0 |
| 20 | 0 | 0 | 5 | 2 | 0 | 0 |
| 21 | 0 | 0 | 10 | 10 | 0 | 0 |
| 22 | 0 | 0 | 0 | 0 | 0 | 0 |
| 23 | 0 | 0 | 0 | 0 | 1 | 2 |
| 24 | 0 | 0 | 0 | 0 | 6 | 0 |
| 25 | 0 | 0 | 0 | 0 | 7 | 4.5 |
| 26 | 0 | 0 | 0 | 0 | 2 | 0.5 |

TABLE 8
Illumina RNA Sequencing Normalized expression of all GOIs

| Row | | | GFP-R1 | GFP-R1 | GFP-R2 | GFP-R2 | GFP-R3 | GFP-R3 | N-R1 | N-R1 |
|---|---|---|---|---|---|---|---|---|---|---|
| Number | Gene | | (CasRx) | (dCasRx) | (CasRx) | (dCasRx) | (CasRx) | (dCasRx) | (CasRx) | (dCasRx) |
| 1 | CasRx | | 456.912 | 209.303 | 347.269 | 279.429 | 452.798 | 257.124 | 87.4314 | 129.998 |
| 2 | GFP | | 297.335 | 671.857 | 126.322 | 704.34 | 263.413 | 645.634 | 252.057 | 188.728 |
| 3 | Notch | | 3.42138 | 4.36542 | 5.24334 | 4.33128 | 3.29456 | 4.83263 | 1.26559 | 5.34223 |
| 4 | white | | 3.38596 | 2.65611 | 2.56199 | 2.2216 | 4.48859 | 2.17292 | 2.42174 | 2.73012 |
| 5 | yellow | | 42.6383 | 92.586 | 19.014 | 125.955 | 28.5648 | 70.0151 | 88.2734 | 100.176 |
| 6 | cinnabar | | 4.38999 | 3.89959 | 2.48593 | 3.14081 | 5.64509 | 3.55941 | 4.45807 | 3.39386 |
| 7 | wingless | | 3.7423 | 4.10564 | 2.44545 | 4.12395 | 2.84169 | 3.31493 | 2.35705 | 3.92515 |
| 8 | GFP_target_1 | | 79.8728 | 25.4987 | 74.1637 | 68.3465 | 95.5171 | 74.957 | 0 | 0 |
| 9 | GFP_target_2 | | 88.095 | 69.0589 | 60.258 | 73.8583 | 85.4627 | 95.3998 | 0 | 0 |
| 10 | GFP_target_3 | | 55.2062 | 57.372 | 66.4383 | 38.5827 | 87.9763 | 40.8856 | 0 | 0 |
| 11 | GFP_target_4 | | 2.3492 | 3.18733 | 0 | 11.0236 | 0 | 6.81427 | 0 | 0 |
| 12 | Notch_target_1 | | 0 | 0 | 0 | 0 | 0 | 0 | 60.155 | 65.0519 |
| 13 | Notch_target_2 | | 0 | 0 | 0 | 0 | 0 | 0 | 22.5581 | 25.1814 |
| 14 | Notch_target_3 | | 0 | 0 | 0 | 0 | 0 | 0 | 13.1589 | 20.9845 |
| 15 | Notch_target_4 | | 0 | 0 | 0 | 0 | 0 | 0 | 0 | 0 |
| 16 | white_target_1 | | 0 | 0 | 0 | 0 | 0 | 0 | 0 | 0 |
| 17 | white_target_2 | | 0 | 0 | 0 | 0 | 0 | 0 | 0 | 0 |
| 18 | white_target_3 | | 0 | 0 | 0 | 0 | 0 | 0 | 0 | 0 |
| 19 | white_target_4 | | 0 | 0 | 0 | 0 | 0 | 0 | 0 | 0 |
| 20 | yellow_target_1 | | 0 | 0 | 0 | 0 | 0 | 0 | 0 | 0 |
| 21 | yellow_target_2 | | 0 | 0 | 0 | 0 | 0 | 0 | 0 | 0 |
| 22 | yellow_target_3 | | 0 | 0 | 0 | 0 | 0 | 0 | 0 | 0 |
| 23 | yellow_target_4 | | 0 | 0 | 0 | 0 | 0 | 0 | 0 | 0 |
| 24 | cinnabar_target_1 | | 0 | 0 | 0 | 0 | 0 | 0 | 0 | 0 |
| 25 | cinnabar_target_2 | | 0 | 0 | 0 | 0 | 0 | 0 | 0 | 0 |
| 26 | cinnabar_target_3 | | 0 | 0 | 0 | 0 | 0 | 0 | 0 | 0 |
| 27 | cinnabar_target_4 | | 0 | 0 | 0 | 0 | 0 | 0 | 0 | 0 |
| 28 | wingless_target_1 | | 0 | 0 | 0 | 0 | 0 | 0 | 0 | 0 |
| 29 | wingless_target_2 | | 0 | 0 | 0 | 0 | 0 | 0 | 0 | 0 |
| 30 | wingless_target_3 | | 0 | 0 | 0 | 0 | 0 | 0 | 0 | 0 |
| 31 | wingless_target_4 | | 0 | 0 | 0 | 0 | 0 | 0 | 0 | 0 |
| | Row | N-R2 | N-R2 | N-R3 | N-R3 | w-R1 | w-R1 | w-R2 | w-R2 | w-R3 |
| | Number | (CasRx) | (dCasRx) | (CasRx) | (dCasRx) | (CasRx) | (dCasRx) | (CasRx) | (dCasRx) | (CasRx) |
| | 1 | 181.037 | 156.417 | 136.334 | 131.55 | 147.878 | 168.128 | 231.087 | 107.751 | 228.481 |
| | 2 | 271.873 | 222.348 | 220.784 | 248.806 | 40.8227 | 17.0346 | 19.5489 | 17.6452 | 12.2819 |
| | 3 | 2.41371 | 6.25379 | 3.11058 | 3.66528 | 6.68648 | 10.8575 | 13.0407 | 7.94586 | 15.7775 |
| | 4 | 4.48885 | 2.60953 | 3.38043 | 2.67727 | 13.8036 | 21.3006 | 11.5516 | 19.2817 | 13.3689 |
| | 5 | 108.79 | 50.1479 | 52.8791 | 65.8647 | 21.644 | 18.0684 | 25.9878 | 18.1843 | 28.0757 |
| | 6 | 6.92318 | 4.56535 | 6.17986 | 3.75379 | 0.27565 | 0.43309 | 0.31221 | 0.42167 | 0.36827 |
| | 7 | 3.5728 | 4.1344 | 3.01987 | 2.94645 | 1.79174 | 2.689 | 2.0913 | 2.45229 | 2.32604 |
| | 8 | 0 | 0 | 0 | 2.55973 | 0 | 0 | 0 | 0 | 0 |
| | 9 | 0 | 0 | 0 | 0 | 0.32894 | 0 | 0 | 0 | 0 |
| | 10 | 0 | 0 | 0 | 0 | 0.32894 | 0 | 0 | 0 | 0 |
| | 11 | 0 | 0 | 0 | 0 | 0.32894 | 0 | 0 | 0 | 0 |
| | 12 | 111.586 | 112.638 | 92.1829 | 84.471 | 0 | 0 | 0 | 0 | 0 |
| | 13 | 47.2095 | 45.9746 | 30.2475 | 35.8362 | 0 | 0 | 0 | 0 | 0 |
| | 14 | 32.1883 | 34.4809 | 27.3668 | 38.3959 | 0 | 0 | 0 | 0 | 0 |
| | 15 | 2.14589 | 2.29873 | 2.88071 | 5.11946 | 0.32894 | 0 | 0 | 0 | 0 |
| | 16 | 0 | 0 | 0 | 0 | 13.4864 | 16.1506 | 11.4639 | 9.06104 | 17.5786 |
| | 17 | 0 | 0 | 0 | 0 | 57.5639 | 35.5313 | 20.0618 | 10.5712 | 57.1303 |
| | 18 | 0 | 0 | 0 | 0 | 23.3545 | 16.1506 | 17.1958 | 7.55087 | 13.1839 |
| | 19 | 0 | 0 | 0 | 0 | 27.9596 | 3.23012 | 20.0618 | 21.1424 | 8.78928 |
| | 20 | 0 | 0 | 0 | 0 | 0 | 0 | 0 | 0 | 0 |
| | 21 | 0 | 0 | 0 | 0 | 0 | 0 | 0 | 0 | 0 |
| | 22 | 0 | 0 | 0 | 0 | 0.32894 | 0 | 0 | 0 | 0 |
| | 23 | 0 | 0 | 0 | 0 | 0 | 0 | 0 | 0 | 0 |
| | 24 | 0 | 0 | 0 | 0 | 0 | 0 | 0 | 0 | 0 |
| | 25 | 0 | 0 | 0 | 0 | 0 | 0 | 0 | 0 | 0 |
| | 26 | 0 | 0 | 0 | 0 | 0 | 0 | 0 | 0 | 0 |
| | 27 | 0 | 0 | 0 | 0 | 0 | 0 | 0 | 0 | 0 |
| | 28 | 0 | 0 | 0 | 0 | 0.32894 | 0 | 0 | 0 | 0 |
| | 29 | 0 | 0 | 0 | 0 | 0.32894 | 0 | 0 | 0 | 0 |
| | 30 | 0 | 0 | 0 | 0 | 0 | 0 | 0 | 0 | 0 |
| | 31 | 0 | 0 | 0 | 0 | 0.32894 | 0 | 0 | 0 | 0 |

| Row | | w-R3 | y-R1 | y-R1 | y-R2 | y-R2 |
|---|---|---|---|---|---|---|
| Number | Gene | (dCasRx) | (CasRx) | (dCasRx) | (CasRx) | (dCasRx) |
| 1 | CasRx | 139.8759205 | 67.53354135 | 25.83680679 | 86.0685427 | 40.44460927 |
| 2 | GFP | 28.24378489 | 24.12459907 | 11.26778644 | 39.27308486 | 16.74077096 |
| 3 | Notch | 8.171755194 | 6.216590861 | 6.785955002 | 5.170985245 | 5.893733944 |
| 4 | white | 15.28473299 | 3.322736419 | 2.30810312 | 3.055549583 | 2.259271397 |
| 5 | yellow | 21.03084425 | 56.44302983 | 72.97088321 | 50.79410167 | 80.51389816 |
| 6 | cinnabar | 0.227116943 | 3.70371127 | 2.285256368 | 4.399294067 | 3.115984533 |
| 7 | wingless | 2.307960752 | 6.979062575 | 5.343554575 | 6.20744743 | 5.913137691 |
| 8 | GFP_target_1 | 0 | | 0 | 0 | 0 |
| 9 | GFP_target_2 | 0 | | 0.254866598 | 0 | 0 |
| 10 | GFP_target_3 | 0 | | 0.254866598 | 0 | 0 |
| 11 | GFP_target_4 | 0 | | 0.254866598 | 0 | 0 |
| 12 | Notch_target_1 | 0 | | 0 | 0 | 0 |
| 13 | Notch_target_2 | 0 | | 0 | 0 | 0 |
| 14 | Notch_target_3 | 0 | | 0 | 0 | 0 |
| 15 | Notch_target_4 | 0 | | 0.254866598 | 0 | 0 |
| 16 | white_target_1 | 0 | | 0.254866598 | 0 | 0 |
| 17 | white_target_2 | 23.23081874 | | 0 | 0 | 0 |
| 18 | white_target_3 | 11.61540937 | | 0.254866598 | 0 | 0 |
| 19 | white_target_4 | 3.871803123 | | 0 | 0 | 0 |
| 20 | yellow_target_1 | 0 | 13.1714765 | 7.645997943 | 7.645375126 | 7.537313939 |
| 21 | yellow_target_2 | 0 | 7.317486946 | 0 | 7.645375126 | 5.024875959 |
| 22 | yellow_target_3 | 0 | 16.09847128 | 10.44953052 | 26.75881294 | 18.84328485 |
| 23 | yellow_target_4 | 0 | 1.463497389 | 0 | 6.371145938 | 6.281094949 |
| 24 | cinnabar_target_1 | 0 | | 0 | 0 | 0 |
| 25 | cinnabar_target_2 | 0 | | 0 | 0 | 0 |
| 26 | cinnabar_target_3 | 0 | | 0 | 0 | 0 |
| 27 | cinnabar_target_4 | 0 | | 0 | 0 | 0 |
| 28 | wingless_target_1 | 0 | | 0.254866598 | 0 | 0 |
| 29 | wingless_target_2 | 0 | | 0.254866598 | 0 | 0 |
| 30 | wingless_target_3 | 0 | | 0 | 0 | 0 |
| 31 | wingless_target_4 | 0 | | 0.254866598 | 0 | 0 |

| Row | y-R3 | y-R3 | cn-R1 | cn-R1 | cn-R2 | cn-R2 |
|---|---|---|---|---|---|---|
| Number | (CasRx) | (dCasRx) | (CasRx) | (dCasRx) | (CasRx) | (dCasRx) |
| 1 | 72.48190139 | 47.08655474 | 228.1725621 | 165.2057063 | 296.7059376 | 225.2311743 |
| 2 | 84.47293343 | 20.91309311 | 18.36587656 | 17.84618193 | 15.59954876 | 17.11425909 |
| 3 | 3.394520041 | 5.374383897 | 104.2329646 | 91.41275352 | 115.6436386 | 81.66572758 |
| 4 | 2.744537783 | 2.826031818 | 2.932166985 | 2.802240594 | 3.367333174 | 3.105287808 |
| 5 | 43.44371436 | 80.5326268 | 5.22817093 | 7.078164921 | 9.250460389 | 18.44976787 |
| 6 | 5.06101688 | 2.999212797 | 27.20798081 | 33.83317835 | 25.13586051 | 24.33554919 |
| 7 | 4.724410363 | 5.530076651 | 71.05775055 | 59.92719762 | 69.21276381 | 54.61807605 |
| 8 | 0 | 0 | 0 | 0 | 0 | 0 |
| 9 | 0 | 0 | 0 | 0 | 0 | 0 |
| 10 | 0 | 0 | 0 | 0 | 0 | 0 |
| 11 | 0 | 0 | 0 | 0 | 0 | 0 |
| 12 | 0 | 0 | 0 | 0 | 0 | 0 |
| 13 | 0 | 0 | 0 | 0 | 0 | 0 |
| 14 | 0 | 0 | 0 | 0 | 0 | 0 |
| 15 | 0 | 0 | 0 | 0 | 0 | 0 |
| 16 | 0 | 0 | 0 | 0 | 0 | 0 |
| 17 | 0 | 0 | 0 | 0 | 0 | 0 |
| 18 | 0 | 0 | 0 | 0 | 0 | 0 |
| 19 | 0 | 0 | 0 | 0 | 0 | 0 |
| 20 | 2.445133661 | 4.86947021 | 0 | 0 | 0 | 0 |
| 21 | 14.67080197 | 0 | 0 | 0 | 0 | 0 |
| 22 | 17.11593563 | 10.95630797 | 0 | 0 | 0 | 0 |
| 23 | 4.890267322 | 1.217367552 | 0 | 0 | 0 | 0 |
| 24 | 0 | 0 | 8.879176964 | 0 | 6.478501124 | 23.37257306 |
| 25 | 0 | 0 | 11.83890262 | 5.526934908 | 19.43550337 | 32.13728796 |
| 26 | 0 | 0 | 23.67780524 | 27.63467454 | 22.67475394 | 43.82357449 |
| 27 | 0 | 0 | 0 | 0 | 0 | 2.921571633 |
| 28 | 0 | 0 | 0 | 0 | 0 | 0 |
| 29 | 0 | 0 | 0 | 0 | 0 | 0 |
| 30 | 0 | 0 | 0 | 0 | 0 | 0 |
| 31 | 0 | 0 | 0 | 0 | 0 | 0 |

| Row | wg-R1 | wg-R1 | wg-R2 | wg-R2 | wg-R3 | wg-R3 |
|---|---|---|---|---|---|---|
| Number | (CasRx) | (dCasRx) | (CasRx) | (dCasRx) | (CasRx) | (dCasRx) |
| 1 | 142.7686257 | 242.2031821 | 392.7726329 | 465.3581939 | 322.4951807 | 400.7910187 |
| 2 | 12.96483796 | 19.39454814 | 4.344017484 | 6.270862507 | 8.103968092 | 7.614372662 |
| 3 | 62.29829119 | 95.70352027 | 31.99051314 | 61.75358482 | 56.90169326 | 61.52866088 |
| 4 | 1.486820128 | 1.394111874 | 0.596563626 | 0.884495643 | 1.103354977 | 0.548502732 |
| 5 | 7.296932936 | 6.790705645 | 1.417454184 | 2.180740618 | 5.475412608 | 1.582529963 |
| 6 | 33.2316186 | 40.47415055 | 10.32856894 | 4.26156739 | 28.1497291 | 7.799157281 |
| 7 | 56.88453641 | 81.05676994 | 21.48941845 | 29.122988 | 41.39936046 | 31.82105058 |
| 8 | 0 | 0 | 0 | 0 | 0 | 0 |
| 9 | 0 | 0 | 0 | 0 | 0 | 0 |
| 10 | 0 | 0 | 0 | 0 | 0 | 0 |
| 11 | 0 | 0 | 0 | 0 | 0 | 0 |
| 12 | 0 | 0 | 0 | 0 | 0 | 0 |
| 13 | 0 | 0 | 0 | 0 | 0 | 0 |
| 14 | 0 | 0 | 0 | 0 | 0 | 0 |
| 15 | 0 | 0 | 0 | 0 | 0 | 0 |
| 16 | 0 | 0 | 0 | 0 | 0 | 0 |
| 17 | 0 | 0 | | | | |
| 18 | 0 | 0 | 0 | 0 | 0 | 0 |
| 19 | 0 | 0 | 0 | 0 | 0 | 0 |
| 20 | 0 | 0 | 0 | 0 | 0 | 0 |
| 21 | 0 | 0 | 0 | 0 | 0 | 0 |
| 22 | 0 | 0 | 0 | 0 | 0 | 0 |
| 23 | 0 | 0 | 0 | 0 | 0 | 0 |
| 24 | 0 | 0 | 0 | 0 | 0 | 0 |
| 25 | 0 | 0 | 0 | 0 | 0 | 0 |
| 26 | 0 | 0 | 0 | 0 | 0 | 0 |
| 27 | 0 | 0 | 0 | 0 | 0 | 0 |
| 28 | 0 | 0 | 2.751211073 | 6.304041674 | 5.76686868 | 3.071615299 |
| 29 | 3.041134831 | 2.779007652 | 2.751211073 | 6.304041674 | 14.4171717 | 6.143230598 |
| 30 | 0 | 0 | 2.751211073 | 15.76010418 | 28.8343434 | 30.71615299 |
| 31 | 0 | 2.779007652 | 2.751211073 | 0 | 0 | 0 |

TABLE 9
GFP DESeq2

| ID | baseMean | log2FoldChange | lfcSE | stat | pvalue | padj |
|---|---|---|---|---|---|---|
| CasRx | 17091.26 | −0.18993 | 0.267202 | −0.71079 | 4.77E−01 | 5.81E−01 |
| GFP | 6746.331 | 1.906909 | 0.392551 | 4.858138 | 1.18E−06 | 7.84E−06 |
| GFP_target_1 | 28.21504 | −0.01673 | 0.442714 | −0.03779 | 0.969854 | 0.978567 |
| GFP_target_2 | 33.33111 | 0.532025 | 0.384234 | 1.384482 | 0.166211 | 0.258516 |
| GFP_target_3 | 23.38727 | 0.000317 | 0.425993 | 0.000745 | 0.999405 | 0.999405 |
| GFP_target_4 | 1.806065 | 1.246814 | 0.695352 | 1.848261 | 0.064565 | NA |
| cinnabar_target_1 | 0 | NA | NA | NA | NA | NA |
| cinnabar_target_2 | 0 | NA | NA | NA | NA | NA |
| cinnabar_target_3 | 0 | NA | NA | NA | NA | NA |
| cinnabar_target_4 | 0 | NA | NA | NA | NA | NA |
| notch_target_1 | 0 | NA | NA | NA | NA | NA |
| notch_target_2 | 0 | NA | NA | NA | NA | NA |
| notch_target_3 | 0 | NA | NA | NA | NA | NA |
| notch_target_4 | 0 | NA | NA | NA | NA | NA |
| white_target_1 | 0 | NA | NA | NA | NA | NA |
| white_target_2 | 0 | NA | NA | NA | NA | NA |
| white_target_3 | 0 | NA | NA | NA | NA | NA |
| white_target_4 | 0 | NA | NA | NA | NA | NA |
| wingless_target_1 | 0 | NA | NA | NA | NA | NA |
| wingless_target_2 | 0 | NA | NA | NA | NA | NA |
| wingless_target_3 | 0 | NA | NA | NA | NA | NA |
| wingless_target_4 | 0 | NA | NA | NA | NA | NA |
| y_target_1 | 0 | NA | NA | NA | NA | NA |
| y_target_2 | 0 | NA | NA | NA | NA | NA |
| y_target_3 | 0 | NA | NA | NA | NA | NA |
| y_target_4 | 0 | NA | NA | NA | NA | NA |

TABLE 10
Notch DESeq2

| ID | baseMean | log2FoldChange | lfcSE | stat | pvalue | padj |
|---|---|---|---|---|---|---|
| CasRx | 7448.501 | −0.08444 | 0.175666 | −0.48068 | 6.31E−01 | 9.99E−01 |
| GFP | 3280.908 | −0.29499 | 0.181961 | −1.6213 | 0.104954 | 0.787327 |
| GFP_target_1 | 0.176733 | 0.008824 | 0.044955 | 0.196295 | 0.844379 | NA |
| GFP_target_2 | 0 | NA | NA | NA | NA | NA |
| GFP_target_3 | 0 | NA | NA | NA | NA | NA |
| GFP_target_4 | 0 | NA | NA | NA | NA | NA |
| cinnabar_target_1 | 0 | NA | NA | NA | NA | NA |
| cinnabar_target_2 | 0 | NA | NA | NA | NA | NA |
| cinnabar_target_3 | 0 | NA | NA | NA | NA | NA |
| cinnabar_target_4 | 0 | NA | NA | NA | NA | NA |
| notch_target_1 | 37.8237 | −0.0996 | 0.213601 | −0.46617 | 0.641096 | 0.999119 |
| notch_target_2 | 14.75352 | −0.02293 | 0.20929 | −0.10941 | 0.912879 | 0.999119 |
| notch_target_3 | 11.71621 | 0.082709 | 0.203764 | 0.405951 | 0.684779 | 0.999119 |
| notch_target_4 | 0.856913 | 0.01865 | 0.082388 | 0.229236 | 0.818686 | NA |
| white_target_1 | 0 | NA | NA | NA | NA | NA |
| white_target_2 | 0 | NA | NA | NA | NA | NA |
| white_target_3 | 0 | NA | NA | NA | NA | NA |
| white_target_4 | 0 | NA | NA | NA | NA | NA |
| wingless_target_1 | 0 | NA | NA | NA | NA | NA |
| wingless_target_2 | 0 | NA | NA | NA | NA | NA |
| wingless_target_3 | 0 | NA | NA | NA | NA | NA |
| wingless_target_4 | 0 | NA | NA | NA | NA | NA |
| y_target_1 | 0 | NA | NA | NA | NA | NA |
| y_target_2 | 0 | NA | NA | NA | NA | NA |
| y_target_3 | 0 | NA | NA | NA | NA | NA |
| y_target_4 | 0 | NA | NA | NA | NA | NA |

TABLE 11
yellow DESeq2

| ID | baseMean | log2FoldChange | lfcSE | stat | pvalue | padj |
|---|---|---|---|---|---|---|
| CasRx | 2788.213 | −0.6048 | 0.128424 | −4.72135 | 2.34E−06 | 2.07E−03 |
| GFP | 413.3289 | −0.43836 | 0.117182 | −3.89026 | 0.0001 | 0.039278 |
| GFP_target_1 | 0 | NA | NA | NA | NA | NA |
| GFP_target_2 | 0 | NA | NA | NA | NA | NA |
| GFP_target_3 | 0 | NA | NA | NA | NA | NA |
| GFP_target_4 | 0 | NA | NA | NA | NA | NA |
| cinnabar_target_1 | 0 | NA | NA | NA | NA | NA |
| cinnabar_target_2 | 0 | NA | NA | NA | NA | NA |
| cinnabar_target_3 | 0 | NA | NA | NA | NA | NA |
| cinnabar_target_4 | 0 | NA | NA | NA | NA | NA |
| notch_target_1 | 0 | NA | NA | NA | NA | NA |
| notch_target_2 | 0 | NA | NA | NA | NA | NA |
| notch_target_3 | 0 | NA | NA | NA | NA | NA |
| notch_target_4 | 0 | NA | NA | NA | NA | NA |
| white_target_1 | 0 | NA | NA | NA | NA | NA |
| white_target_2 | 0 | NA | NA | NA | NA | NA |
| white_target_3 | 0 | NA | NA | NA | NA | NA |
| white_target_4 | 0 | NA | NA | NA | NA | NA |
| wingless_target_1 | 0 | NA | NA | NA | NA | NA |
| wingless_target_2 | 0 | NA | NA | NA | NA | NA |
| wingless_target_3 | 0 | NA | NA | NA | NA | NA |
| wingless_target_4 | 0 | NA | NA | NA | NA | NA |
| y_target_1 | 2.710062 | −0.00389 | 0.052573 | −0.07431 | 0.940767 | 0.999958 |
| y_target_2 | 2.204002 | −0.05547 | 0.042342 | −1.4442 | 0.148684 | 0.999958 |
| y_target_3 | 6.171777 | −0.05452 | 0.074414 | −0.73583 | 0.461836 | 0.999958 |
| y_target_4 | 0.988176 | −0.01077 | 0.02709 | −0.40674 | 0.684199 | 0.999958 |

TABLE 12
white DESeq2

| ID | baseMean | log2FoldChange | lfcSE | stat | pvalue | padj |
|---|---|---|---|---|---|---|
| CasRx | 6203.838 | −0.35205 | 0.16269 | −2.16411 | 3.05E−02 | 4.86E−01 |
| GFP | 212.6639 | −0.06243 | 0.187843 | −0.33247 | 0.739537 | 0.997726 |
| GFP_target_1 | 0 | NA | NA | NA | NA | NA |
| GFP_target_2 | 0 | NA | NA | NA | NA | NA |
| GFP_target_3 | 0 | NA | NA | NA | NA | NA |
| GFP_target_4 | 0 | NA | NA | NA | NA | NA |
| cinnabar_target_1 | 0 | NA | NA | NA | NA | NA |
| cinnabar_target_2 | 0 | NA | NA | NA | NA | NA |
| cinnabar_target_3 | 0 | NA | NA | NA | NA | NA |
| cinnabar_target_4 | 0 | NA | NA | NA | NA | NA |
| notch_target_1 | 0 | NA | NA | NA | NA | NA |
| notch_target_2 | 0 | NA | NA | NA | NA | NA |
| notch_target_3 | 0 | NA | NA | NA | NA | NA |
| notch_target_4 | 0 | NA | NA | NA | NA | NA |
| white_target_1 | 3.243738 | −0.06813 | 0.123661 | −0.55043 | 0.582028 | NA |
| white_target_2 | 9.644613 | −0.19197 | 0.160494 | −1.20852 | 0.226849 | 0.974247 |
| white_target_3 | 4.236898 | −0.10482 | 0.146424 | −0.71625 | 0.473835 | 0.997726 |
| white_target_4 | 4.175768 | −0.09298 | 0.12305 | −0.76142 | 0.446404 | 0.997726 |
| wingless_target_1 | 0 | NA | NA | NA | NA | NA |
| wingless_target_2 | 0 | NA | NA | NA | NA | NA |
| wingless_target_3 | 0 | NA | NA | NA | NA | NA |
| wingless_target_4 | 0 | NA | NA | NA | NA | NA |
| y_target_1 | 0 | NA | NA | NA | NA | NA |
| y_target_2 | 0 | NA | NA | NA | NA | NA |
| y_target_3 | 0 | NA | NA | NA | NA | NA |
| y_target_4 | 0 | NA | NA | NA | NA | NA |

TABLE 13
cinnabar DESeq2

| ID | baseMean | log2FoldChange | lfcSE | stat | pvalue | padj |
|---|---|---|---|---|---|---|
| CasRx | 9756.891 | −0.31977 | 0.155077 | −2.06205 | 3.92E−02 | 7.81E−01 |
| GFP | 185.5797 | 0.050602 | 0.203188 | 0.249045 | 0.803326 | 0.999917 |
| GFP_target_1 | 0 | NA | NA | NA | NA | NA |
| GFP_target_2 | 0 | NA | NA | NA | NA | NA |
| GFP_target_3 | 0 | NA | NA | NA | NA | NA |
| GFP_target_4 | 0 | NA | NA | NA | NA | NA |
| cinnabar_target_1 | 3.167913 | 0.009321 | 0.052546 | 0.179028 | 0.857915 | NA |
| cinnabar_target_2 | 5.714305 | 0.010921 | 0.082527 | 0.132512 | 0.894579 | NA |
| cinnabar_target_3 | 9.972621 | 0.05305 | 0.111683 | 0.48013 | 0.631135 | 0.999917 |
| cinnabar_target_4 | 0.235448 | 0.009011 | 0.032904 | 0.270431 | 0.786829 | NA |
| notch_target_1 | 0 | NA | NA | NA | NA | NA |
| notch_target_2 | 0 | NA | NA | NA | NA | NA |
| notch_target_3 | 0 | NA | NA | NA | NA | NA |
| notch_target_4 | 0 | NA | NA | NA | NA | NA |
| white_target_1 | 0 | NA | NA | NA | NA | NA |
| white_target_2 | 0 | NA | NA | NA | NA | NA |
| white_target_3 | 0 | NA | NA | NA | NA | NA |
| white_target_4 | 0 | NA | NA | NA | NA | NA |
| wingless_target_1 | 0 | NA | NA | NA | NA | NA |
| wingless_target_2 | 0 | NA | NA | NA | NA | NA |
| wingless_target_3 | 0 | NA | NA | NA | NA | NA |
| wingless_target_4 | 0 | NA | NA | NA | NA | NA |
| y_target_1 | 0 | NA | NA | NA | NA | NA |
| y_target_2 | 0 | NA | NA | NA | NA | NA |
| y_target_3 | 0 | NA | NA | NA | NA | NA |
| y_target_4 | 0 | NA | NA | NA | NA | NA |

TABLE 14
wingless DESeq2

| ID | baseMean | log2FoldChange | lfcSE | stat | pvalue | padj |
|---|---|---|---|---|---|---|
| CasRx | 14840.17 | 0.148283 | 0.234807 | 0.63263 | 5.27E−01 | 0.999744 |
| GFP | 96.33487 | 0.194755 | 0.244254 | 0.796583 | 0.425693 | 0.999744 |
| GFP_target_1 | 0 | NA | NA | NA | NA | NA |
| GFP_target_2 | 0 | NA | NA | NA | NA | NA |
| GFP_target_3 | 0 | NA | NA | NA | NA | NA |
| GFP_target_4 | 0 | NA | NA | NA | NA | NA |
| cinnabar_target_1 | 0 | NA | NA | NA | NA | NA |
| cinnabar_target_2 | 0 | NA | NA | NA | NA | NA |
| cinnabar_target_3 | 0 | NA | NA | NA | NA | NA |
| cinnabar_target_4 | 0 | NA | NA | NA | NA | NA |
| notch_target_1 | 0 | NA | NA | NA | NA | NA |
| notch_target_2 | 0 | NA | NA | NA | NA | NA |
| notch_target_3 | 0 | NA | NA | NA | NA | NA |
| notch_target_4 | 0 | NA | NA | NA | NA | NA |
| white_target_1 | 0 | NA | NA | NA | NA | NA |
| white_target_2 | 0 | NA | NA | NA | NA | NA |
| white_target_3 | 0 | NA | NA | NA | NA | NA |
| white_target_4 | 0 | NA | NA | NA | NA | NA |
| wingless_target_1 | 1.10424 | 0.011389 | 0.096477 | 0.120494 | 0.904092 | 0.999744 |
| wingless_target_2 | 2.014782 | −0.02467 | 0.141635 | −0.17636 | 0.860007 | 0.999744 |
| wingless_target_3 | 4.765334 | 0.042922 | 0.108104 | 0.406848 | 0.68412 | 0.999744 |
| wingless_target_4 | 0.304993 | 0.001149 | 0.057736 | 0.017859 | 0.985751 | 0.999744 |
| y_target_1 | 0 | NA | NA | NA | NA | NA |
| y_target_2 | 0 | NA | NA | NA | NA | NA |
| y_target_3 | 0 | NA | NA | NA | NA | NA |
| y_target_4 | 0 | NA | NA | NA | NA | NA |

TABLE 15
Primers used to generate the constructs in this study

| | Descrip- | | | |
|---|---|---|---|---|
| Construct | tion | Primer | Primer Sequence (5′ to 3′) | PCR Template |
| OA-1050E | CasRx | 1050E.C3 | TACTAATTTTCCACATCTCTATTTTGACCCGCAGATTAATTAATGA | pNLS-RfxCas13d- |
| | | | GCCCCAAGAAGAA | NLS-HA (pCasRx) |
| | | 1050E.C4 | CAATTGATTTGTTATTTTAAAAACGATTCATTCTAGCTAGCTTAAG | |
| | | | CGTAATCTGGAACA | |
| OA-1050R | dCasRx | 1050E.C3 | TACTAATTTTCCACATCTCTATTTTGACCCGCAGATTAATTAATGA | pNLS-dRfxCas13d- |
| | | | GCCCCAAGAAGAA | NLS-HA (pdCasRx) |
| | | 1050E.C4 | CAATTGATTTGTTATTTTAAAAACGATTCATTCTAGCTAGCTTAAG | |
| | | | CGTAATCTGGAACA | |
| OA-1050L | UASt | 1041.C9 | GCGGGTTCTCGACGGTCACGGCGGGCATGTCGACGCGGCCGCAACC | pJFRC81 |
| | promoter | | AACAACACTAGTAG | |
| | | 1041.C11 | CTGGCCTCCACCTTTCTCTTCTTCTTGGGGCTCATGTTTAAACCCA | |
| | | | ATTCCCTATTCAGA | |
| | CasRx | 1050L.C1 | AATACAAGAAGAGAACTCTGAATAGGGAATTGGGTTTAAACATGAG | pCasRx |
| | | | CCCCAAGAAGAA | |
| | | 1050E.C4 | CAATTGATTTGTTATTTTAAAAACGATTCATTCTAGCTAGCTTAAG | |
| | | | CGTAATCTGGAACA | |
| OA-1050S | UASt | 1041.C9 | GCGGGTTCTCGACGGTCACGGCGGGCATGTCGACGCGGCCGCAACC | pJFRC81 |
| | promoter | | AACAACACTAGTAG | |
| | | 1041.C11 | CTGGCCTCCACCTTTCTCTTCTTCTTGGGGCTCATGTTTAAACCCA | |
| | | | ATTCCCTATTCAGA | |
| | dCasRx | 1050L.C1 | AATACAAGAAGAGAACTCTGAATAGGGAATTGGGTTTAAACATGAG | pdCasRx |
| | | | CCCCAAGAAGAA | |
| | | 1050E.C4 | CAATTGATTTGTTATTTTAAAAACGATTCATTCTAGCTAGCTTAAG | |
| | | | CGTAATCTGGAACA | |
| OA-1043 | U6:3 | 1043.C1 | GGGAATTGGGAATTGGGCAATATTTAAATGGCGGCGCGCCGAATTC | Addgene plasmid |
| | promoter | | TTTTTTGCTCACCT | #112688 |
| | | 1043.C23 | ACACTAGTGGATCTCTAGAGGTACCGTTGCGGCCGCAAAAAAGTTG | |
| | | | TAATAGCCCCTCAAAACTGGACCTTCCACAACTGCAGCCGACGTTA | |
| | | | AATTGAAA | |
| OA-1052B | Ubiq | 1052B.C1 | GGGAATTGGGCAATATTTAAATGGCGGCTGCAGCGCGCAGATCGCC | Addgene plasmid |
| | promoter | | GAT | #112686 |
| | | 1052B.C2 | TTTCTTTATGTTTTTGGCGTCTTCCATCCTAGGTCTGCGGGTCAAA | |
| | | | ATAGAGATG | |
| | T2A-eGFP | 908A1 | ATAAAGGCCAAGAAGGGCGGAAAGATCGCCGTGGAGGGCAGAGGAA | Addgene plasmid |
| | | | GTCTTCTAACATGC | #112686 |
| | | 908A2 | TTGTTATTTTAAAAACGATTCATTCTAGGCGATCGCTTACTTGTAC | |
| | | | AGCTCGTCCATGCC | |
| | Reversed | 908A3 | ACCGTGACCTACATCGTCGACACTAGTGGATCTCTAGACGCGCAGA | Addgene plasmid |
| | Ubiq | | TCGCCGATG | #112686 |
| | | 908A4 | GGATCATAAACTTTCGAAGTCATGCGGCCGCTCTGCGGGTCAAAAT | |
| | | | AGAGATGT | |

1. A nucleic acid molecule comprising:
(a) a sequence encoding a Cas13 polypeptide; and
(b) a sequence encoding a guide RNA comprising one or more spacers and one or more Cas13-specific direct repeats, wherein the spacers are capable of specifically hybridizing with one or more target RNAs.

2. The nucleic acid molecule of claim 1, wherein the Cas13 is Cas13d.

3. (canceled)

4. The nucleic acid molecule of claim 1, wherein the sequence encoding the Cas13 polypeptide further comprises a localization signal.

5. (canceled)

6. The nucleic acid molecule of claim 1, wherein the target RNA is an endogenous RNA or a viral RNA.

7. (canceled)

8. The nucleic acid molecule of claim 1, wherein the spacers are positioned between two Cas13-specific direct repeats.

9. The nucleic acid molecule of claim 1, wherein the spacers are 20 to 40 nucleotides in length.

10.-11. (canceled)

12. The nucleic acid molecule of claim 1, wherein the Cas13-specific direct repeats are 25 to 45 nucleotides in length.

13.-14. (canceled)

15. The nucleic acid molecule of claim 1, wherein the guide RNA further comprises a AAAAC motif at its 5′ end.

16. The nucleic acid molecule of claim 1, wherein the guide RNA comprises two or more spacers, and wherein the two or more spacers are capable of specifically hybridizing with the same target RNA.

17. The nucleic acid molecule of claim 1, wherein the guide RNA comprises two or more spacers, and wherein the two or more spacers are capable of specifically hybridizing with different target RNAs.

18. (canceled)

19. The nucleic acid molecule of claim 1, wherein the sequence encoding a Cas13 polypeptide is operably linked to a ubiquitous promoter, an inducible promoter, or a tissue-specific promoter.

20.-21. (canceled)

22. A vector comprising the nucleic acid molecule of claim 1.

23.-24. (canceled)

25. A cell comprising the nucleic acid molecule of claim 1.

26. A method of modifying a target RNA in a cell, the method comprising contacting the cell with the nucleic acid molecule of claim 1.

27. A method of modifying a target RNA in a cell, the method comprising contacting the cell with the vector of claim 22.

28. (canceled)

29. A method of modifying a target RNA in a cell, the method comprising contacting the cell with (a) a nucleic acid molecule comprising a sequence encoding a Cas13 polypeptide, and (b) a sequence encoding a guide RNA comprising one or more spacers and one or more Cas13-specific direct repeats, wherein the one or more spacers are capable of specifically hybridizing with the target RNA.

30. The method of claim 29, wherein the Cas13 is Cas13d.

31.-47. (canceled)

48. The method of claim 29, wherein the nucleic acid molecule is comprised within a first vector and the sequence encoding the guide RNA is comprised within a second vector.

49. (canceled)

50. A transgenic organism having a recombinant nucleic acid molecule stably integrated into the genome of the organism, wherein the recombinant nucleic acid molecule comprises a sequence that encodes a Cas13 polypeptide.

51. (canceled)

52. The transgenic organism of claim 50, wherein the Cas13 polypeptide is a Cas13d.

53.-56. (canceled)


SEQUENCE LISTING (23 sequences)

SEQ ID NO: 1 - protein, 7 aa, Artificial sequence
  synthetic polypeptide
PKKKRRV

SEQ ID NO: 2 - protein, 16 aa, Artificial sequence
  synthetic polypeptide
KRPAATKKAGQAKKKK

SEQ ID NO: 3 - RNA, 36 nt, Artificial sequence
  synthetic nucleotide
caaguaaaccccuaccaacuggucgggguuugaaac

SEQ ID NO: 4 - DNA, 59 bp, Artificial sequence
  synthetic primer
tactaattttccacatctctattttgacccgcagattaattaatgagccccaagaagaa

SEQ ID NO: 5 - DNA, 60 bp, Artificial sequence
  synthetic primer
caattgatttgttattttaaaaacgattcattctagctagcttaagcgtaatctggaaca

SEQ ID NO: 6 - DNA, 59 bp, Artificial sequence; identical to SEQ ID NO: 4
  synthetic primer

SEQ ID NO: 7 - DNA, 60 bp, Artificial sequence; identical to SEQ ID NO: 5
  synthetic primer

SEQ ID NO: 8 - DNA, 60 bp, Artificial sequence
  synthetic primer
gcgggttctcgacggtcacggcgggcatgtcgacgcggccgcaaccaacaacactagtag

SEQ ID NO: 9 - DNA, 60 bp, Artificial sequence
  synthetic primer
ctggcctccacctttctcttcttcttggggctcatgtttaaacccaattccctattcaga

SEQ ID NO: 10 - DNA, 58 bp, Artificial sequence
  synthetic primer
aatacaagaagagaactctgaatagggaattgggtttaaacatgagccccaagaagaa

SEQ ID NO: 11 - DNA, 60 bp, Artificial sequence; identical to SEQ ID NO: 5
  synthetic primer

SEQ ID NO: 12 - DNA, 60 bp, Artificial sequence; identical to SEQ ID NO: 8
  synthetic primer

SEQ ID NO: 13 - DNA, 60 bp, Artificial sequence; identical to SEQ ID NO: 9
  synthetic primer

SEQ ID NO: 14 - DNA, 58 bp, Artificial sequence; identical to SEQ ID NO: 10
  synthetic primer

SEQ ID NO: 15 - DNA, 60 bp, Artificial sequence; identical to SEQ ID NO: 5
  synthetic primer

SEQ ID NO: 16 - DNA, 60 bp, Artificial sequence
  synthetic primer
gggaattgggaattgggcaatatttaaatggcggcgcgccgaattcttttttgctcacct

SEQ ID NO: 17 - DNA, 100 bp, Artificial sequence
  synthetic primer
acactagtggatctctagaggtaccgttgcggccgcaaaaaagttgtaatagcccctcaaaactggaccttccacaactgcagccgacgttaaattgaaa

SEQ ID NO: 18 - DNA, 49 bp, Artificial sequence
  synthetic primer
gggaattgggcaatatttaaatggcggctgcagcgcgcagatcgccgat

SEQ ID NO: 19 - DNA, 55 bp, Artificial sequence
  synthetic primer
tttctttatgtttttggcgtcttccatcctaggtctgcgggtcaaaatagagatg

SEQ ID NO: 20 - DNA, 60 bp, Artificial sequence
  synthetic primer
ataaaggccaagaagggcggaaagatcgccgtggagggcagaggaagtcttctaacatgc

SEQ ID NO: 21 - DNA, 60 bp, Artificial sequence
  synthetic primer
ttgttattttaaaaacgattcattctaggcgatcgcttacttgtacagctcgtccatgcc

SEQ ID NO: 22 - DNA, 55 bp, Artificial sequence
  synthetic primer
accgtgacctacatcgtcgacactagtggatctctagacgcgcagatcgccgatg

SEQ ID NO: 23 - DNA, 54 bp, Artificial sequence
  synthetic primer
ggatcataaactttcgaagtcatgcggccgctctgcgggtcaaaatagagatgt